SEQUENCE LISTING (174 sequences)

SEQ ID NO: 1 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 2 - protein, 213 aa, synthetic construct
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 454 aa, synthetic construct
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLCTPSRGS

SEQ ID NO: 4 - DNA, 645 bp, synthetic construct
  source 1..645: mol_type = other DNA
gacatccagatgacccagtctccttccaccctgtctgcatctgtaggagacagagtcaccatcacttgcagcgccagcagcagtattagttacatgcactggtatcagcagaaaccagggaaagcccctaagctcctgatctataccacctccaacttggccagtggggtcccagccaggttcagcggcagtggatctgggacagaattcactctcaccatcagcagcctgcagcctgatgattttgcaacttattactgccaccagaggagcacctatcccctgactttcggccaggggaccaaggtggaggtgaaacgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 5 - DNA, 1368 bp, synthetic construct
  source 1..1368: mol_type = other DNA
caggtgcagctggtgcagtctggggctgaggtgaagaagcctgggagctcagtgaaggtttcctgcaaggcatctggatacaccttcaccagctacaggatgcactgggtgcgacaggcccctggacaagggcttgagtggatcggatacatcaaccctagtaccggttacacagagtacaaccagaagttcaaggacaaggccaccatcaccgccgacgagtccacgaacacagcctacatggagctgagcagcctgagatctgaggacacggccgtgtattactgtgcgagaggcggaggagtgttcgactactggggccagggaaccctggtgaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccaagagaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggtccttatgtaccccttctagaggatcctgatga

SEQ ID NO: 6 - protein, 5 aa, synthetic construct
SYRMH

SEQ ID NO: 7 - protein, 17 aa, synthetic construct
YINPSTGYTEYNQKFKD

SEQ ID NO: 8 - protein, 7 aa, synthetic construct
GGGVFDY

SEQ ID NO: 9 - protein, 10 aa, synthetic construct
SASSSISYMH

SEQ ID NO: 10 - protein, 7 aa, synthetic construct
YTSILHS

SEQ ID NO: 11 - protein, 9 aa, synthetic construct
QQGNTLPWT

SEQ ID NO: 12 - protein, 116 aa, synthetic construct
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 13 - protein, 106 aa, synthetic construct
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 14-15 - skipped (2 entries)

SEQ ID NO: 16 - protein, 6 aa, synthetic construct
LCTPSR

SEQ ID NO: 17 - protein, 6 aa, synthetic construct
MCTPSR

SEQ ID NO: 18 - protein, 6 aa, synthetic construct
VCTPSR

SEQ ID NO: 19 - protein, 6 aa, synthetic construct
LCSPSR

SEQ ID NO: 20 - protein, 6 aa, synthetic construct
LCAPSR

SEQ ID NO: 21 - protein, 6 aa, synthetic construct
LCVPSR

SEQ ID NO: 22 - protein, 6 aa, synthetic construct
LCGPSR

SEQ ID NO: 23 - protein, 6 aa, synthetic construct
ICTPAR

SEQ ID NO: 24 - protein, 6 aa, synthetic construct
LCTPSK

SEQ ID NO: 25 - protein, 6 aa, synthetic construct
MCTPSK

SEQ ID NO: 26 - protein, 6 aa, synthetic construct
VCTPSK

SEQ ID NO: 27 - protein, 6 aa, synthetic construct
LCSPSK

SEQ ID NO: 28 - protein, 6 aa, synthetic construct
LCAPSK

SEQ ID NO: 29 - protein, 6 aa, synthetic construct
LCVPSK

SEQ ID NO: 30 - protein, 6 aa, synthetic construct
LCGPSK

SEQ ID NO: 31 - protein, 6 aa, synthetic construct
LCTPSA

SEQ ID NO: 32 - protein, 6 aa, synthetic construct
ICTPAA

SEQ ID NO: 33 - protein, 6 aa, synthetic construct
MCTPSA

SEQ ID NO: 34 - protein, 6 aa, synthetic construct
VCTPSA

SEQ ID NO: 35 - protein, 6 aa, synthetic construct
LCSPSA

SEQ ID NO: 36 - protein, 6 aa, synthetic construct
LCAPSA

SEQ ID NO: 37 - protein, 6 aa, synthetic construct
LCVPSA

SEQ ID NO: 38 - protein, 6 aa, synthetic construct
LCGPSA

SEQ ID NO: 39 - protein, 12 aa, synthetic construct
  MOD_RES 6: note = FORMYLATION
SPGSLGTPSRGS

SEQ ID NO: 40 - protein, 8 aa, synthetic construct
SLSLSPGK

SEQ ID NO: 41 - skipped

SEQ ID NO: 42 - protein, 6 aa, synthetic construct
KVDNAL

SEQ ID NO: 43 - protein, 6 aa, synthetic construct
QSGNSQ

SEQ ID NO: 44 - protein, 17 aa, synthetic construct
  MOD_RES 7: note = FORMYLATION
KVDNALGTPSRQSGNSQ

SEQ ID NO: 45 - skipped

SEQ ID NO: 46 - protein, 6 aa, synthetic construct
SWNSGA

SEQ ID NO: 47 - protein, 6 aa, synthetic construct
GVHTFP

SEQ ID NO: 48 - protein, 18 aa, synthetic construct
  MOD_RES 8: note = FORMYLATION
SWNSGALGTPSRGVHTFP

SEQ ID NO: 49 - protein, 6 aa, Homo sapiens
ASTKGP

SEQ ID NO: 50 - protein, 7 aa, Homo sapiens
KSTSGGT

SEQ ID NO: 51 - protein, 4 aa, Homo sapiens
PEPV

SEQ ID NO: 52 - protein, 8 aa, Homo sapiens
NSGALTSG

SEQ ID NO: 53 - protein, 21 aa, Homo sapiens
NSGALTSGVHTFPAVLQSSGL

SEQ ID NO: 54 - protein, 5 aa, Homo sapiens
QSSGL

SEQ ID NO: 55 - protein, 4 aa, Homo sapiens
TQTY

SEQ ID NO: 56 - protein, 5 aa, Homo sapiens
HKPSN

SEQ ID NO: 57 - protein, 22 aa, Homo sapiens
EPKSCDKTHTCPPCPAPELLGG

SEQ ID NO: 58 - protein, 5 aa, Homo sapiens
FPPKP

SEQ ID NO: 59 - protein, 5 aa, Homo sapiens
ISRTP

SEQ ID NO: 60 - protein, 9 aa, Homo sapiens
DVSHEDPEV

SEQ ID NO: 61 - protein, 7 aa, Homo sapiens
SHEDPEV

SEQ ID NO: 62 - protein, 9 aa, Homo sapiens
DGVEVHNAK

SEQ ID NO: 63 - protein, 5 aa, Homo sapiens
QYNST

SEQ ID NO: 64 - protein, 5 aa, Homo sapiens
VLTVL

SEQ ID NO: 65 - protein, 7 aa, Homo sapiens
NKALPAP

SEQ ID NO: 66 - protein, 9 aa, Homo sapiens
SKAKGQPRE

SEQ ID NO: 67 - protein, 7 aa, Homo sapiens
KAKGQPR

SEQ ID NO: 68 - protein, 10 aa, Homo sapiens
PPSRKELTKN

SEQ ID NO: 69 - protein, 5 aa, Homo sapiens
YPSDI

SEQ ID NO: 70 - protein, 7 aa, Homo sapiens
NGQPENN

SEQ ID NO: 71 - protein, 10 aa, Homo sapiens
TPPVLDSDGS

SEQ ID NO: 72 - protein, 19 aa, Homo sapiens
HEALHNHYTQKSLSLSPGK

SEQ ID NO: 73 - protein, 6 aa, Homo sapiens
SLSPGK

SEQ ID NO: 74 - protein, 12 aa, Homo sapiens
PCSRSTSESTAA

SEQ ID NO: 75 - protein, 5 aa, Homo sapiens
FPEPV

SEQ ID NO: 76 - protein, 12 aa, Homo sapiens
SGALTSGVHTFP

SEQ ID NO: 77 - protein, 6 aa, Homo sapiens
QSSGLY

SEQ ID NO: 78 - protein, 5 aa, Homo sapiens
VAGPS

SEQ ID NO: 79 - protein, 5 aa, Homo sapiens
VLTVV

SEQ ID NO: 80 - protein, 7 aa, Homo sapiens
NKGLPAP

SEQ ID NO: 81 - protein, 9 aa, Homo sapiens
SKTKGQPRE

SEQ ID NO: 82 - protein, 5 aa, Homo sapiens
MTKNQ

SEQ ID NO: 83 - skipped

SEQ ID NO: 84 - protein, 10 aa, Homo sapiens
TPPMLDSDGS

SEQ ID NO: 85 - protein, 4 aa, Homo sapiens
GNVF

SEQ ID NO: 86 - skipped

SEQ ID NO: 87 - protein, 10 aa, Homo sapiens
PCSRSTSGGT

SEQ ID NO: 88 - protein, 19 aa, Homo sapiens
SGALTSGVHTFPAVLQSSG

SEQ ID NO: 89 - protein, 10 aa, Homo sapiens
RVELKTPLGD

SEQ ID NO: 90 - protein, 7 aa, Homo sapiens
CPRCPKP

SEQ ID NO: 91 - protein, 21 aa, Homo sapiens
PKSCDTPPPCPRCPAPELLGG

SEQ ID NO: 92 - protein, 10 aa, Homo sapiens
PPSREEMTKN

SEQ ID NO: 93 - protein, 8 aa, Homo sapiens
SSGQPENN

SEQ ID NO: 94 - skipped

SEQ ID NO: 95 - protein, 7 aa, Homo sapiens
HEALHNR

SEQ ID NO: 96 - protein, 5 aa, Homo sapiens
STKGP

SEQ ID NO: 97 - protein, 11 aa, Homo sapiens
CPAPEFLGGPS

SEQ ID NO: 98 - protein, 9 aa, Homo sapiens
DVSQEDPEV

SEQ ID NO: 99 - protein, 7 aa, Homo sapiens
NKGLPSS

SEQ ID NO: 100 - protein, 10 aa, Homo sapiens
SKAKGQPREP

SEQ ID NO: 101 - protein, 10 aa, Homo sapiens
PPSQEEMTKN

SEQ ID NO: 102 - protein, 19 aa, Homo sapiens
HEALHNHYTQKSLSLSLGK

SEQ ID NO: 103 - protein, 13 aa, Homo sapiens
ASPTSPKVFPLSL

SEQ ID NO: 104 - protein, 5 aa, Homo sapiens
QPDGN

SEQ ID NO: 105 - protein, 10 aa, Homo sapiens
VQGFFPQEPL

SEQ ID NO: 106 - protein, 11 aa, Homo sapiens
SGQGVTARNFP

SEQ ID NO: 107 - protein, 7 aa, Homo sapiens
SGDLYTT

SEQ ID NO: 108 - protein, 4 aa, Homo sapiens
PATQ

SEQ ID NO: 109 - protein, 4 aa, Homo sapiens
HRPA

SEQ ID NO: 110 - protein, 5 aa, Homo sapiens
LLGSE

SEQ ID NO: 111 - protein, 8 aa, Homo sapiens
GLRDASGV

SEQ ID NO: 112 - protein, 10 aa, Homo sapiens
SSGKSAVQGP

SEQ ID NO: 113 - protein, 4 aa, Homo sapiens
GCYS

SEQ ID NO: 114 - protein, 4 aa, Homo sapiens
CAEP

SEQ ID NO: 115 - protein, 24 aa, Homo sapiens
SGNTFRPEVHLLPPPSEELALNEL

SEQ ID NO: 116 - protein, 5 aa, Homo sapiens
ARGFS

SEQ ID NO: 117 - protein, 11 aa, Homo sapiens
QGSQELPREKY

SEQ ID NO: 118 - protein, 4 aa, Homo sapiens
AAED

SEQ ID NO: 119 - protein, 4 aa, Homo sapiens
HEAL

SEQ ID NO: 120 - protein, 25 aa, Homo sapiens
IDRLAGKPTHVNVSVVMAEVDGTCY

SEQ ID NO: 121 - skipped

SEQ ID NO: 122 - protein, 11 aa, synthetic construct
NSGALCTPSRG

SEQ ID NO: 123 - protein, 5 aa, synthetic construct
CTPSR

SEQ ID NO: 124 - skipped

SEQ ID NO: 125 - protein, 9 aa, synthetic construct
NLCTPSRAP

SEQ ID NO: 126 - protein, 10 aa, synthetic construct
KAKGLCTPSR

SEQ ID NO: 127 - protein, 6 aa, Homo sapiens
RTVAAP

SEQ ID NO: 128 - protein, 5 aa, Homo sapiens
YPREA

SEQ ID NO: 129 - protein, 4 aa, Homo sapiens
PREA

SEQ ID NO: 130 - protein, 8 aa, Homo sapiens
DNALQSGN

SEQ ID NO: 131 - protein, 9 aa, Homo sapiens
TEQDSKDST

SEQ ID NO: 132 - protein, 6 aa, Homo sapiens
HQGLSS

SEQ ID NO: 133 - protein, 4 aa, Homo sapiens
RGEC

SEQ ID NO: 134 - protein, 6 aa, Homo sapiens
QPKAAP

SEQ ID NO: 135 - protein, 7 aa, Homo sapiens
DFYPGAV

SEQ ID NO: 136 - protein, 8 aa, Homo sapiens
DSSPVKAG

SEQ ID NO: 137 - protein, 6 aa, Homo sapiens
APTECS

SEQ ID NO: 138 - protein, 99 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVE

SEQ ID NO: 139 - protein, 8 aa, synthetic construct
GALTSGVH

SEQ ID NO: 140 - protein, 11 aa, synthetic construct
GALCTPSRGVH

SEQ ID NO: 141 - protein, 9 aa, synthetic construct
SLCTPSRGS

SEQ ID NO: 142 - protein, 18 aa, synthetic construct
KVDNALLCTPSRQSGNSQ

SEQ ID NO: 143 - skipped

SEQ ID NO: 144 - protein, 6 aa, synthetic construct
  MOD_RES 2: note = FORMYLATION
LGTPSR

SEQ ID NO: 145 - protein, 7 aa, synthetic construct
SLSLSPG

SEQ ID NO: 146-150 - skipped (5 entries)

SEQ ID NO: 151 - protein, 16 aa, synthetic construct
  MOD_RES 7: note = FORMYLATION
KVDNALTPSRQSGNSQ

SEQ ID NO: 152-153 - skipped (2 entries)

SEQ ID NO: 154 - protein, 6 aa, synthetic construct
  VARIANT 2: note = Wherein X can be C or S
LXTPSR

SEQ ID NO: 155-157 - skipped (3 entries)

SEQ ID NO: 158 - protein, 10 aa, synthetic construct
QKSLSLSPGK

SEQ ID NO: 159-161 - skipped (3 entries)

SEQ ID NO: 162 - protein, 107 aa, Homo sapiens [sequence omitted]

SEQ ID NO: 163 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 330 aa, Homo sapiens [sequence omitted]

SEQ ID NO: 165 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 166 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 167 - protein, 326 aa, Homo sapiens
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 168 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPDRDLCGCYSVSSVLSGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 169 - skipped

SEQ ID NO: 170 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 171 - protein, 107 aa, Homo sapiens
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 172 - protein, 107 aa, Mus musculus
RADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 173 - protein, 105 aa, Rattus norvegicus
QPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 174 - skipped

(19) United States
(12) Patent Application Publication
(10) Pub. No.: US 2024/0360123 A1
(43) Pub. Date: Oct. 31, 2024

(54) Anti-CD25 Antibody-Maytansine Conjugates and Methods of Use Thereof

(71) Applicant: R.P. Scherer Technologies, LLC, Carson City, NV (US)

(72) Inventors: David Rabuka, Kensington, CA (US); Penelope M. Drake, Castro Valley, CA (US)

(21) Appl. No.: 18/384,707

(22) Filed: Oct. 27, 2023

Related U.S. Application Data
(63) Continuation of application No. 17/519,183, filed on Nov. 4, 2021, now Pat. No. 11,857,637.
Application No. 17/519,183 is a continuation of application No. PCT/US2019/054753, filed on Oct. 4, 2019.

(51) Int. Cl. C07D 471/04; A61K 47/65; A61K 47/68; C07K 16/28

(52) U.S. Cl. CPC C07D 471/04; A61K 47/68033; A61K 47/6807; C07K 16/2866; A61K 47/65

(57) ABSTRACT

The present disclosure provides anti-CD25 antibody-maytansine conjugate structures. The disclosure also encompasses methods of production of such conjugates, as well as methods of using the same.

### CROSS-REFERENCE TO RELATED APPLICATIONS

This application is a continuation of U.S. application Ser. No. 17/519,183, filed Nov. 4, 2021, now issued as U.S. Pat. No. 11,857,637, which is a continuation of International Application No. PCT/US2019/054753, filed Oct. 4, 2019, the disclosures of which are incorporated by reference herein in their entirety.

### INCORPORATION-BY-REFERENCE OF MATERIAL ELECTRONICALLY SUBMITTED

A Sequence Listing is provided herewith as a Sequence Listing XML, “RDWD-033CON2_SEQ_LIST” created on May 10, 2024 and having a size of 142,336 bytes. The contents of the Sequence Listing XML are incorporated by reference herein in their entirety.

### INTRODUCTION

The field of protein-small molecule therapeutic conjugates has advanced greatly, providing a number of clinically beneficial drugs with the promise of providing more in the years to come. Protein-conjugate therapeutics can provide several advantages, due to, for example, specificity, multiplicity of functions and relatively low off-target activity, resulting in fewer side effects. Chemical modification of proteins may extend these advantages by rendering them more potent, stable, or multimodal.

A number of standard chemical transformations are commonly used to create and manipulate post-translational modifications on proteins. There are a number of methods where one is able to modify the side chains of certain amino acids selectively. For example, carboxylic acid side chains (aspartate and glutamate) may be targeted by initial activation with a water-soluble carbodiimide reagent and subsequent reaction with an amine. Similarly, lysine can be targeted through the use of activated esters or isothiocyanates, and cysteine thiols can be targeted with malcimides and α-halo-carbonyls.

One significant obstacle to the creation of a chemically altered protein therapeutic or reagent is the production of the protein in a biologically active, homogenous form. Conjugation of a drug or detectable label to a polypeptide can be difficult to control, resulting in a heterogeneous mixture of conjugates that differ in the number of drug molecules attached and in the position of chemical conjugation. In some instances, it may be desirable to control the site of conjugation and/or the drug or detectable label conjugated to the polypeptide using the tools of synthetic organic chemistry to direct the precise and selective formation of chemical bonds on a polypeptide.

Interleukin-2 (IL-2) and its receptor (IL-2R) play an important role as signaling molecules in cellular immune responses. This ligand-receptor interaction has been considered a target in the clinical treatment of acute allograft rejection, graft-versus-host disease, T-cell-mediated autoimmune disease, and certain hematologic malignancies. The IL-2R is composed of the α, β, and γ_{c} subunits. Upon activation, they assemble to form the intermediate-affinity IL-2R (βγ_{c}) or the high-affinity IL-2R (αβγ_{c}), in which the IL-2 interaction with the α chain initiates the sequential recruitment of the β and γ_{c} subunits. T cells express the α chain (CD25) upon activation. CD25 is expressed on Hodgkin lymphoma (HL) cells and various T and B cell non-Hodgkin lymphoma (NHL) cells. CD25 is expressed on nearly 100% of adult T-cell leukemia lymphoma cells and hairy cell leukemia cells. CD25 is also up-regulated in refractory Acute Myeloid Leukemia (AML). In addition, CD25 is expressed on regulatory T cells.

### SUMMARY

The present disclosure provides anti-CD25 antibody-maytansine conjugate structures. The disclosure also encompasses methods of production of such conjugates, as well as methods of using the same.

Aspects of the present disclosure include a conjugate having at least one modified amino acid residue with a side chain of formula (I):

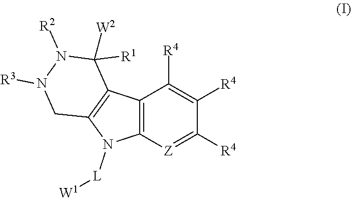

wherein
Z is CR⁴ or N;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is a linker comprising -(T¹-V¹)ₐ-(T²-V²)_{b}-(T³-V³)_{c}-(T⁴-V⁴)_{d}-, wherein a, b, c and d are each independently 0 or 1, where the sum of a, b, c and d is 1 to 4;
T¹, T², T³ and T⁴ are each independently selected from (C₁-C₁₂)alkyl, substituted (C₁-C₁₂)alkyl, (EDA)_{w}, (PEG)ₙ, (AA)ₚ, —(CR¹³OH)ₕ-, piperidin-4-amino (4AP), an acetal group, a hydrazine, a disulfide, and an ester, wherein EDA is an ethylene diamine moiety, PEG is a polyethylene glycol or a modified polyethylene glycol, and AA is an amino acid residue, wherein w is an integer from 1 to 20, n is an integer from 1 to 30, p is an integer from 1 to 20, and h is an integer from 1 to 12;
V¹, V², V³ and V⁴ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
each R¹³ is independently selected from hydrogen, an alkyl, a substituted alkyl, an aryl, and a substituted aryl;
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl;
W¹ is a maytansinoid; and
W² is an anti-CD25 antibody.

In some instances, the conjugate includes the following, where:
T¹ is selected from a (C₁-C₁₂)alkyl and a substituted (C₁-C₁₂)alkyl;
T², T³ and T⁴ are each independently selected from (EDA)_{w}, (PEG)ₙ, (C₁-C₁₂)alkyl, substituted (C₁-C₁₂)alkyl, (AA)ₚ, —(CR¹³OH)ₕ—, 4-amino-piperidine (4AP), an acetal group, a hydrazine, and an ester; and
V¹, V², V³ and V⁴ are each independently selected from the group consisting of a covalent bond, —CO—, —NR¹⁵—, —NR¹⁵(CH₂)_{q}—, —NR¹⁵(C₆H₄)—, —CONR¹⁵, —NR¹⁵CO—, —C(O)O—, —OC(O)—, —O—, —S—, —S(O)—, —SO₂—, —SO₂NR¹⁵, —NR¹⁵SO₂—, and —P(O)OH—;
wherein:
(PEG)ₙ is

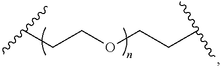

where n is an integer from 1 to 30;
EDA is an ethylene diamine moiety having the following structure:

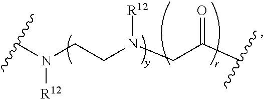

where y is an integer from 1 to 6 and r is 0 or 1;
4-amino-piperidine (4AP) is

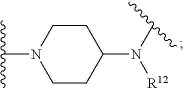

each R¹² and R¹⁵ is independently selected from hydrogen, an alkyl, a substituted alkyl, a polyethylene glycol moiety, an aryl and a substituted aryl, wherein any two adjacent R¹² groups may be cyclically linked to form a piperazinyl ring; and
R¹³ is selected from hydrogen, an alkyl, a substituted alkyl, an aryl, and a substituted aryl.

In some instances, the conjugate includes the following, where: T¹, T², T³ and T⁴, and V¹, V², V³ and V⁴ are selected from the following table:

| T¹ | V¹ | T² | V² | T³ | V³ | T⁴ | V⁴ |
|---|---|---|---|---|---|---|---|
| (C₁—C₁₂)alkyl | —CONR¹⁵— | (PEG)ₙ | —CO— | — | — | — | — |
| (C₁—C₁₂)alkyl | —CO— | (AA)ₚ | —NR¹⁵— | (PEG)ₙ | —CO— | — | — |
| (C₁—C₁₂)alkyl | —CO— | (AA)ₚ | — | — | — | — | — |
| (C₁—C₁₂)alkyl | —CONR¹⁵— | (PEG)ₙ | —NR¹⁵— | — | — | — | — |
| (C₁—C₁₂)alkyl | —CO— | (AA)ₚ | —NR¹⁵— | (PEG)ₙ | —NR¹⁵— | — | — |
| (C₁—C₁₂)alkyl | —CO— | (EDA)_{w} | —CO— | — | — | — | — |
| (C₁—C₁₂)alkyl | —CONR¹⁵— | (C₁—C₁₂)alkyl | —NR¹⁵— | — | — | — | — |
| (C₁—C₁₂)alkyl | —CONR¹⁵— | (PEG)ₙ | —CO— | (EDA)_{w} | — | — | — |
| (C₁—C₁₂)alkyl | —CO— | (EDA)_{w} | — | — | — | — | — |
| (C₁—C₁₂)alkyl | —CO— | (EDA)_{w} | —CO— | (CR¹³OH)ₕ | —CONR¹⁵— | (C₁—C₁₂)alkyl | —CO— |
| (C₁—C₁₂)alkyl | —CO— | (AA)ₚ | —NR¹⁵— | (C₁—C₁₂)alkyl | —CO— | — | — |
| (C₁—C₁₂)alkyl | —CONR¹⁵— | (PEG)ₙ | —CO— | (AA)ₚ | — | — | — |
| (C₁—C₁₂)alkyl | —CO— | (EDA)_{w} | —CO— | (CR¹³OH)ₕ | —CO— | (AA)ₚ | — |
| (C₁—C₁₂)alkyl | —CO— | (AA)ₚ | —NR¹⁵— | (C₁—C₁₂)alkyl | —CO— | (AA)ₚ | — |
| (C₁—C₁₂)alkyl | —CO— | (AA)ₚ | —NR¹⁵— | (PEG)ₙ | —CO— | (AA)ₚ | — |
| (C₁—C₁₂)alkyl | —CO— | (AA)ₚ | —NR¹⁵— | (PEG)ₙ | —SO₂— | (AA)ₚ | — |
| (C₁—C₁₂)alkyl | —CO— | (EDA)_{w} | —CO— | (CR¹³OH)ₕ | —CONR¹⁵— | (PEG)ₙ | —CO— |
| (C₁—C₁₂)alkyl | —CO— | (CR¹³OH)ₕ | —CO— | — | — | — | — |
| (C₁—C₁₂)alkyl | —CONR¹⁵— | substituted | —NR¹⁵— | (PEG)ₙ | —CO— | — | — |
| | | (C₁—C₁₂)alkyl | | | | | |
| (C₁—C₁₂)alkyl | —SO₂— | (C₁—C₁₂)alkyl | —CO— | — | — | — | — |
| (C₁—C₁₂)alkyl | —CONR¹⁵— | (C₁—C₁₂)alkyl | — | (CR¹³OH)ₕ | —CONR¹⁵— | — | — |
| (C₁—C₁₂)alkyl | —CO— | (AA)ₚ | —NR¹⁵— | (PEG)ₙ | —CO— | (AA)ₚ | —NR¹⁵— |
| (C₁—C₁₂)alkyl | —CO— | (AA)ₚ | —NR¹⁵— | (PEG)ₙ | —P(O)OH— | (AA)ₚ | — |
| (C₁—C₁₂)alkyl | —CO— | (EDA)_{w} | — | (AA)ₚ | — | — | — |
| (C₁—C₁₂)alkyl | —CONR¹⁵— | (C₁—C₁₂)alkyl | —NR¹⁵— | — | —CO— | — | — |
| (C₁—C₁₂)alkyl | —CONR¹⁵— | (C₁—C₁₂)alkyl | —NR¹⁵— | — | —CO— | (C₁—C₁₂)alkyl | —NR¹⁵— |
| (C₁—C₁₂)alkyl | —CO— | 4AP | —CO | (C₁—C₁₂)alkyl | —CO— | (AA)ₚ | — |
| (C₁—C₁₂)alkyl | —CO— | 4AP | —CO | (C₁—C₁₂)alkyl | —CO— | — | — |

In some instances, the conjugate includes the following, where: the linker, L, is selected from one of the following structures:

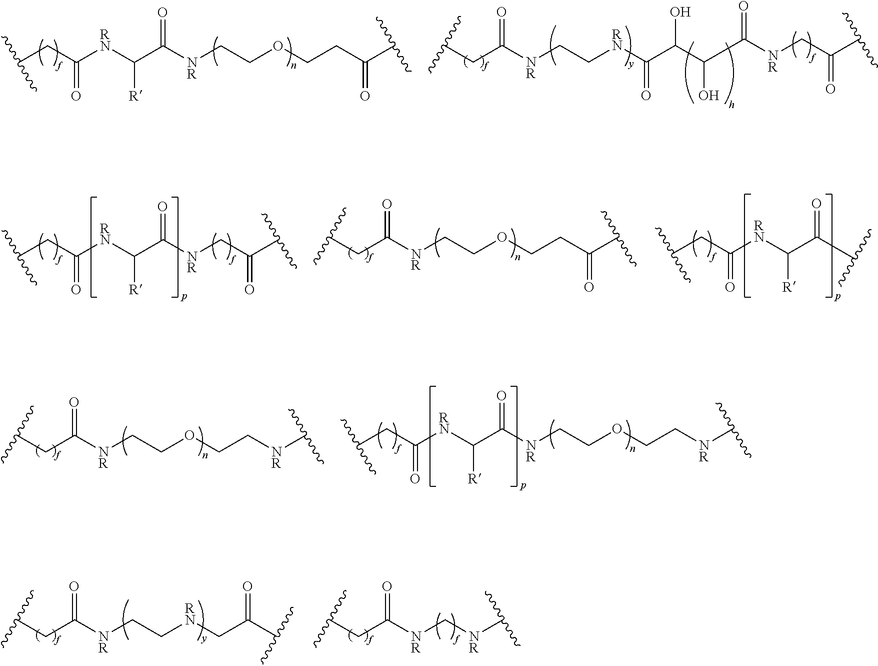

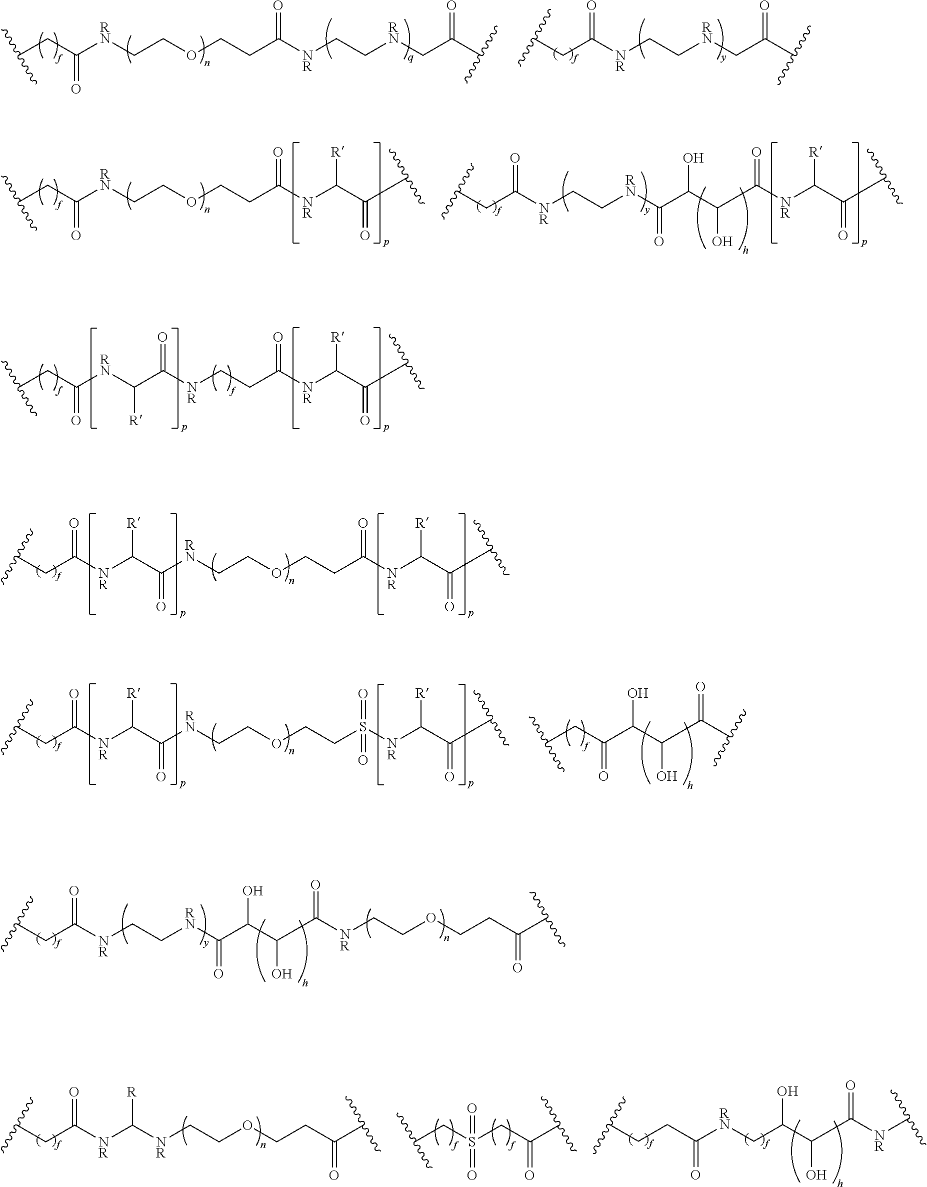

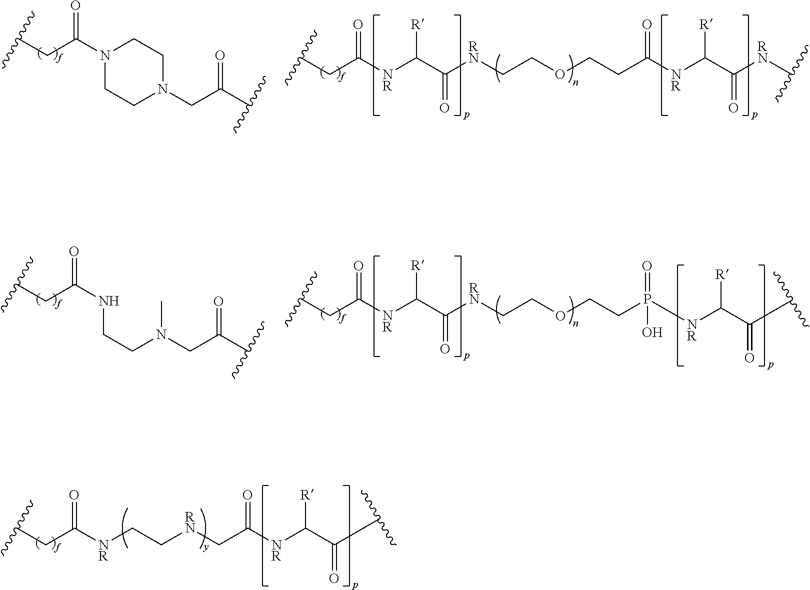

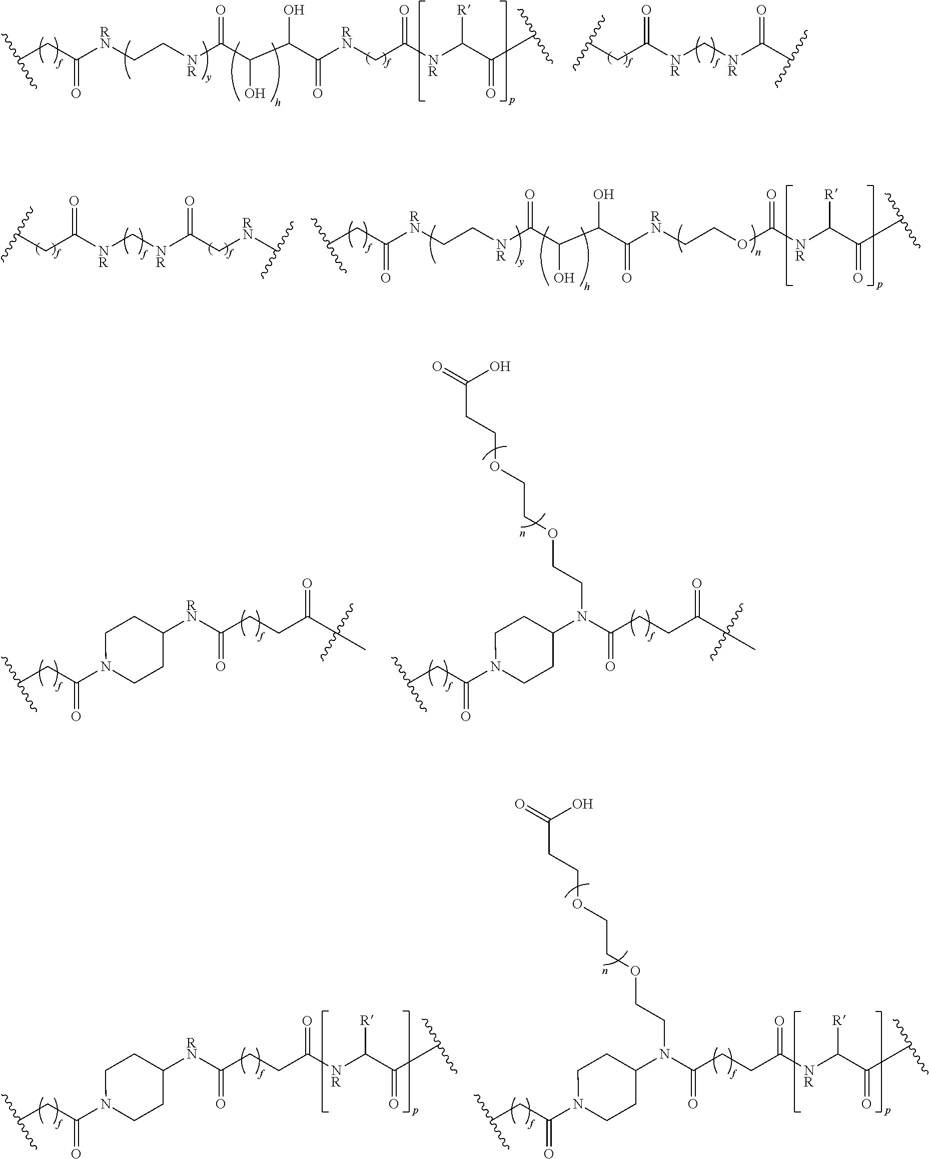

wherein
each f is independently 0 or an integer from 1 to 12;
each y is independently 0 or an integer from 1 to 20;
each n is independently 0 or an integer from 1 to 30;
each p is independently 0 or an integer from 1 to 20;
each h is independently 0 or an integer from 1 to 12;
each R is independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl; and
each R′ is independently H, a sidechain group of an amino acid, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In some instances, the maytansinoid is of the formula:

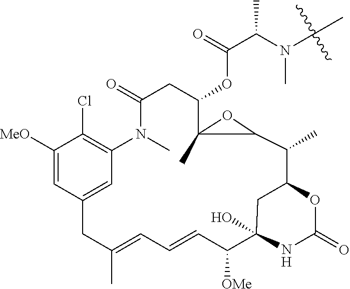

where [symbol] indicates the point of attachment between the maytansinoid and L.

In some instances, the conjugate includes the following, where: T¹ is (C₁-C₁₂)alkyl, V¹ is —CO—, T² is 4AP, V² is —CO—, T³ is (C₁-C₁₂)alkyl, V³ is —CO—, T⁴ is absent and V⁴ is absent.

In some instances, the linker, L, comprises the following structure:

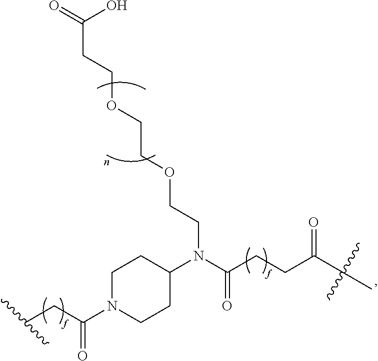

wherein
each f is independently an integer from 1 to 12; and
n is an integer from 1 to 30.

In some instances, the anti-CD25 antibody is an IgG1 antibody.

In some instances, the anti-CD25 antibody is an IgG1 kappa antibody.

In some instances, the anti-CD25 antibody competes with interleukin-2 (IL-2) for binding to CD25.

In some instances, the anti-CD25 antibody binds an epitope within amino acids 101-137, an epitope within amino acids 106-132, an epitope within amino acids 111-127, an epitope comprising amino acids 116-122, or an epitope consisting of amino acids 116-122, of a CD25 amino acid sequence depicted in Table 3.

In some instances, the anti-CD25 antibody competes for binding to CD25 with an antibody comprising the six complementarity determining regions (CDRs) of daclizumab.

In some instances, the anti-CD25 antibody comprises the six CDRs of daclizumab.

In some instances, the anti-CD25 antibody comprises a sequence of the formula (II):

X¹(FGly′)X²X²⁰X³Z³⁰ (II)

wherein
FGly′ is the modified amino acid residue of formula (I);
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid.

In some instances, the sequence is L(FGly′)TPSR.

In some instances, the conjugate includes the following, where:
Z³⁰ is selected from R, K, H, A, G, L, V, I, and P;
X¹ is selected from L, M, S, and V; and
X² and X³ are each independently selected from S, T, A, V, G, and C.

In some instances, the modified amino acid residue is positioned at a C-terminus of a heavy chain constant region of the anti-CD25 antibody.

In some instances, the heavy chain constant region comprises a sequence of the formula (II):

X¹(FGly′)X²X²⁰X³Z³⁰ (II)

wherein
FGly′ is the modified amino acid residue of formula (I);
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and wherein the sequence is C-terminal to the amino acid sequence SLSLSPG.

In some instances, the heavy chain constant region comprises the sequence SPGSL(FGly′)TPSRGS.

In some instances, the conjugate includes the following, where:
Z³⁰ is selected from R, K, H, A, G, L, V, I, and P;
X¹ is selected from L, M, S, and V; and
X² and X³ are each independently selected from S, T, A, V, G, and C.

In some instances, the modified amino acid residue is positioned in a light chain constant region of the anti-CD25 antibody.

In some instances, the light chain constant region comprises a sequence of the formula (II):

X¹(FGly′)X²X²⁰X³Z³⁰ (II)

wherein
FGly′ is the modified amino acid residue of formula (I);
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and
wherein the sequence is C-terminal to the sequence KVDNAL, and/or is N-terminal to the sequence QSGNSQ.

In some instances, the light chain constant region comprises the sequence KVDNAL(FGly′)TPSRQSGNSQ.

In some instances, the conjugate includes the following, where:
Z³⁰ is selected from R, K, H, A, G, L, V, I, and P;
X¹ is selected from L, M, S, and V; and
X² and X³ are each independently selected from S, T, A, V, G, and C.

In some instances, the modified amino acid residue is positioned in a heavy chain CH1 region of the anti-CD25 antibody.

In some instances, the heavy chain CH1 region comprises a sequence of the formula (II):

X¹(FGly′)X²X²⁰X³Z³⁰ (II)

wherein
FGly′ is the modified amino acid residue of formula (I);
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and
wherein the sequence is C-terminal to the amino acid sequence SWNSGA and/or is N-terminal to the amino acid sequence GVHTFP.

In some instances, the heavy chain CH1 region comprises the sequence SWNSGAL(FGly′)TPSRGVHTFP.

In some instances, the conjugate includes the following, where:
Z³⁰ is selected from R, K, H, A, G, L, V, I, and P;
X¹ is selected from L, M, S, and V; and
X² and X³ are each independently selected from S, T, A, V, G, and C.

In some instances, the modified amino acid residue is positioned in a heavy chain CH2 region of the anti-CD25 antibody.

In some instances, the modified amino acid residue is positioned in a heavy chain CH3 region of the anti-CD25 antibody.

Aspects of the present disclosure include a pharmaceutical composition comprising a conjugate according to the present disclosure, and a pharmaceutically-acceptable excipient.

Aspects of the present disclosure include a method comprising administering to a subject an effective amount of a conjugate according to the present disclosure.

Aspects of the present disclosure include a method of treating cancer in a subject. The method includes administering to the subject a therapeutically effective amount of a pharmaceutical composition comprising a conjugate according to the present disclosure, where the administering is effective to treat cancer in the subject.

In some instances, the cancer is a hematologic malignancy.

In some instances, the hematologic malignancy is leukemia. In some instances, the leukemia is acute myeloid leukemia (AML). In some instances, the AML is refractory AML.

In some instances, the hematologic malignancy is lymphoma. In some instances, the lymphoma is Hodgkin lymphoma (HL). In some instances, the lymphoma is Non-Hodgkin lymphoma (NHL).

Aspects of the present disclosure include a method of reducing inhibitory signals derived from T regulatory cells in a subject. The method includes administering to the subject a therapeutically effective amount of a pharmaceutical composition comprising a conjugate according to the present disclosure, where the administering is effective to reduce inhibitory signals derived from T regulatory cells in the subject.

Aspects of the present disclosure include a method of delivering a drug to a target site in a subject. The method includes administering to the subject a pharmaceutical composition comprising a conjugate according to the present disclosure, where the administering is effective to release a therapeutically effective amount of the drug from the conjugate at the target site in the subject.

Aspects of the present disclosure include an anti-CD25 antibody comprising a formylglycine (FGly) residue.

In some instances, the anti-CD25 antibody comprises the sequence:

X¹(FGly)X²X²⁰X³Z³⁰

wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the antibody, X¹ is present; and
X² and X³ are each independently any amino acid.

In some instances, the sequence is L(FGly)TPSR.

In some instances, the anti-CD25 antibody includes the following, where:
Z³⁰ is selected from R, K, H, A, G, L, V, I, and P;
X¹ is selected from L, M, S, and V; and
X² and X³ are each independently selected from S, T, A, V, G, and C.

In some instances, the the sequence is at a C-terminus of a heavy chain constant region of the anti-CD25 antibody.

In some instances, the heavy chain constant region comprises the sequence:

X¹(FGly)X²X²⁰X³Z³⁰

wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid,
wherein the sequence is C-terminal to the amino acid sequence SLSLSPG.

In some instances, the heavy chain constant region comprises the sequence SPGSL(FGly)TPSRGS.

In some instances, the anti-CD25 antibody includes the following, where:
Z³⁰ is selected from R, K, H, A, G, L, V, I, and P;
X¹ is selected from L, M, S, and V; and
X² and X³ are each independently selected from S, T, A, V, G, and C.

In some instances, the FGly residue is positioned in a light chain constant region of the anti-CD25 antibody.

In some instances, the light chain constant region comprises the sequence:

X¹(FGly)X²X²⁰X³Z³⁰ (II)

wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and
wherein the sequence is C-terminal to the sequence KVDNAL, and/or is N-terminal to the sequence QSGNSQ.

In some instances, the light chain constant region comprises the sequence KVDNAL(FGly)TPSRQSGNSQ.

In some instances, the anti-CD25 antibody includes the following, where:
Z³⁰ is selected from R, K, H, A, G, L, V, I, and P;
X¹ is selected from L, M, S, and V; and
X² and X³ are each independently selected from S, T, A, V, G, and C.

In some instances, the FGly residue is positioned in a heavy chain CH1 region of the anti-CD25 antibody.

In some instances, the heavy chain CH1 region comprises the sequence:

X¹(FGly)X²X²⁰X³Z³⁰

wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and
wherein the sequence is C-terminal to the amino acid sequence SWNSGA and/or is N-terminal to the amino acid sequence GVHTFP.

In some instances, the heavy chain CH1 region comprises the sequence SWNSGAL(FGly)TPSRGVHTFP.

In some instances, the anti-CD25 antibody includes the following, where:
Z³⁰ is selected from R, K, H, A, G, L, V, I, and P;
X¹ is selected from L, M, S, and V; and
X² and X³ are each independently selected from S, T, A, V, G, and C.

In some instances, the FGly residue is positioned in a heavy chain CH2 region of the anti-CD25 antibody.

In some instances, the FGly residue is positioned in a heavy chain CH3 region of the anti-CD25 antibody.

Aspects of the present disclosure include a cell comprising the anti-CD25 antibody according to the present disclosure.

Aspects of the present disclosure include a nucleic acid encoding the anti-CD25 antibody according to the present disclosure. Aspects of the present disclosure also include an expression vector comprising the nucleic acid. Aspects of the present disclosure also include a host cell comprising the nucleic acid or the expression vector.

### BRIEF DESCRIPTION OF THE DRAWINGS

FIG. 1, panel A, shows a formylglycine-generating enzyme (FGE) recognition sequence inserted at the desired location along the antibody backbone using standard molecular biology techniques. Upon expression, FGE, which is endogenous to eukaryotic cells, catalyzes the conversion of the Cys within the consensus sequence to a formylglycine residue (FGly). FIG. 1, panel B, shows antibodies carrying aldehyde moieties (2 per antibody) reacted with a Hydrazino-iso-Pictet-Spengler (HIPS) linker and payload to generate a site-specifically conjugated ADC. FIG. 1, panel C, shows HIPS chemistry, which proceeds through an intermediate hydrazonium ion followed by intramolecular alkylation with a nucleophilic indole to generate a stable C—C bond.

FIG. 2 shows a hydrophobic interaction column (HIC) trace of an aldehyde-tagged anti-CD25 antibody conjugated at the heavy chain C-terminus to a maytansine payload attached to a HIPS-4AP linker, according to embodiments of the present disclosure.

FIG. 3 shows a reversed phase chromatography (PLRP) trace of an aldehyde-tagged anti-CD25 antibody conjugated at the heavy chain C-terminus to a maytansine payload attached to a HIPS-4AP linker, according to embodiments of the present disclosure.

FIG. 4 shows a graph of analytical size exclusion chromatography (SEC) analysis of an aldehyde-tagged anti-CD25 antibody conjugated at the heavy chain C-terminus (CT) to a maytansine payload attached to a HIPS-4AP linker, according to embodiments of the present disclosure.

FIG. 5 shows a graph of intact mass analysis of an aldehyde-tagged anti-CD25 antibody conjugated at the heavy chain C-terminus to a maytansine payload attached to a HIPS-4AP linker, according to embodiments of the present disclosure.

FIG. 6 shows in vitro cytotoxicity data for an aldehyde-tagged anti-CD25 antibody conjugated at the heavy chain C-terminus to a maytansine payload attached to a HIPS-4AP linker in SU-DHL-1 cells, according to embodiments of the present disclosure.

FIG. 7 shows in vitro cytotoxicity data for an aldehyde-tagged anti-CD25 antibody conjugated at the heavy chain C-terminus to a maytansine payload attached to a HIPS-4AP linker in HDLM-2 cells, according to embodiments of the present disclosure.

FIG. 8 shows in vitro cytotoxicity data for an aldehyde-tagged anti-CD25 antibody conjugated at the heavy chain C-terminus to a maytansine payload attached to a HIPS-4AP linker in EoL-1 cells, according to embodiments of the present disclosure.

FIG. 9 shows in vitro cytotoxicity data for an aldehyde-tagged anti-CD25 antibody conjugated at the heavy chain C-terminus to a maytansine payload attached to a HIPS-4AP linker in L-540 cells, according to embodiments of the present disclosure.

FIG. 10 shows in vitro cytotoxicity data for an aldehyde-tagged anti-CD25 antibody conjugated at the heavy chain C-terminus to a maytansine payload attached to a HIPS-4AP linker in Karpas 299 cells, according to embodiments of the present disclosure.

FIG. 11 shows data demonstrating the in vivo efficacy of the aldehyde-tagged anti-CD25 antibody conjugated at the heavy chain C-terminus to a maytansine payload attached to a HIPS-4AP linker in a Karpas 299 xenograft model, according to embodiments of the present disclosure.

FIG. 12A depicts a site map showing possible modification sites for generation of an aldehyde tagged Ig polypeptide. The upper sequence is the amino acid sequence of the conserved region of an IgG1 light chain polypeptide (SEQ ID NO: //) and shows possible modification sites in an Ig light chain; the lower sequence is the amino acid sequence of the conserved region of an Ig heavy chain polypeptide (SEQ ID NO: //; GenBank Accession No. AAG00909) and shows possible modification sites in an Ig heavy chain. The heavy and light chain numbering is based on the full-length heavy and light chains.

FIG. 12B depicts an alignment of immunoglobulin heavy chain constant regions for IgG1 (SEQ ID NO: //), IgG2 (SEQ ID NO: //), IgG3 (SEQ ID NO: //), IgG4 (SEQ ID NO: //), and IgA (SEQ ID NO: //), showing modification sites at which aldehyde tags can be provided in an immunoglobulin heavy chain. The heavy and light chain numbering is based on the full-heavy and light chains.

FIG. 12C depicts an alignment of immunoglobulin light chain constant regions (SEQ ID NOs: //), showing modification sites at which aldehyde tags can be provided in an immunoglobulin light chain.

### DEFINITIONS

The following terms have the following meanings unless otherwise indicated. Any undefined terms have their art recognized meanings.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the Ci carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups-alkylene-aryl and -substituted alkylene-aryl where alkylene, substituted alkylene and aryl are defined herein.

“Alkoxy” refers to the group-O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group-NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups-alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group-alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C═CH), and propargyl (—CH₂C═CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group-O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group-O-aryl, wherein aryl is as defined herein, including, by way of example, phenoxy, naphthoxy, and the like, including optionally substituted aryl groups as also defined herein.

“Amino” refers to the group-NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group-N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O— substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group-OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups-alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂-moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group-OC(O) NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group-S-alkyl, wherein alkyl is as defined herein. In certain embodiments, sulfur may be oxidized to —S(O)—. The sulfoxide may exist as one or more stereoisomers.

The term “substituted thioalkoxy” refers to the group-S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, —NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰ trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂ (M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have-H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR ⁸⁰R⁸⁰, —OC(O)R⁷⁰, —OC(S)R⁷⁰, —CO₂⁻M⁺, —CO₂R⁷⁰, —OC(S)OR⁷⁰, —NR⁷⁰C(O)R⁷⁰, —NR⁷⁰C(S) R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

“Patient” refers to human and non-human subjects, especially mammalian subjects.

The term “treating” or “treatment” as used herein means the treating or treatment of a disease or medical condition in a patient, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like.

“Native amino acid sequence” or “parent amino acid sequence” are used interchangeably herein to refer to the amino acid sequence of a polypeptide prior to modification to include a modified amino acid residue.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like.

The terms “amino acid side chain” or “side chain of an amino acid” and the like may be used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

The term “carbohydrate” and the like may be used to refer to monomers units and/or polymers of monosaccharides, disaccharides, oligosaccharides, and polysaccharides. The term sugar may be used to refer to the smaller carbohydrates, such as monosaccharides, disaccharides. The term “carbohydrate derivative” includes compounds where one or more functional groups of a carbohydrate of interest are substituted (replaced by any convenient substituent), modified (converted to another group using any convenient chemistry) or absent (e.g., eliminated or replaced by H). A variety of carbohydrates and carbohydrate derivatives are available and may be adapted for use in the subject compounds and conjugates.

The term “antibody” is used in the broadest sense and includes monoclonal antibodies (including full length monoclonal antibodies), polyclonal antibodies, and multispecific antibodies (e.g., bispecific antibodies), humanized antibodies, single-chain antibodies, chimeric antibodies, antibody fragments (e.g., Fab fragments), and the like. An antibody is capable of binding a target antigen. (Janeway, C., Travers, P., Walport, M., Shlomchik (2001) Immuno Biology, 5th Ed., Garland Publishing, New York). A target antigen can have one or more binding sites, also called epitopes, recognized by complementarity determining regions (CDRs) formed by one or more variable regions of an antibody.

The term “natural antibody” refers to an antibody in which the heavy and light chains of the antibody have been made and paired by the immune system of a multi-cellular organism. Spleen, lymph nodes, bone marrow and serum are examples of tissues that produce natural antibodies. For example, the antibodies produced by the antibody producing cells isolated from a first animal immunized with an antigen are natural antibodies.

The term “humanized antibody” or “humanized immunoglobulin” refers to a non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28 (4/5): 489-498 (1991); Studnicka et al., Protein Engineering 7 (6): 805-814 (1994); Roguska. et al., PNAS 91:969-973 (1994)), and chain shuffling (U.S. Pat. No. 5,565,332). In certain embodiments, framework substitutions are identified by modeling of the interactions of the CDR and framework residues to identify framework residues important for antigen binding and sequence comparison to identify unusual framework residues at particular positions (see, e.g., U.S. Pat. No. 5,585,089; Riechmann et al., Nature 332:323 (1988)). Additional methods for humanizing antibodies contemplated for use in the present invention are described in U.S. Pat. Nos. 5,750,078; 5,502,167; 5,705,154; 5,770,403; 5,698,417; 5,693,493; 5,558,864; 4,935,496; and 4,816,567, and PCT publications WO 98/45331 and WO 98/45332. In particular embodiments, a subject rabbit antibody may be humanized according to the methods set forth in US20040086979 and US20050033031. Accordingly, the antibodies described above may be humanized using methods that are well known in the art.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a mouse antibody and the constant or effector domain from a human antibody, although domains from other mammalian species may be used.

An immunoglobulin polypeptide immunoglobulin light or heavy chain variable region is composed of a framework region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, 1991). The framework region of an antibody, that is the combined framework regions of the constituent light and heavy chains, serves to position and align the CDRs. The CDRs are primarily responsible for binding to an epitope of an antigen.

Throughout the present disclosure, the numbering of the residues in an immunoglobulin heavy chain and in an immunoglobulin light chain is that as in Kabat et al., Sequences of Proteins of Immunological Interest, 5th Ed. Public Health Service, National Institutes of Health, Bethesda, Md. (1991), expressly incorporated herein by reference.

A “parent Ig polypeptide” is a polypeptide comprising an amino acid sequence which lacks an aldehyde-tagged constant region as described herein. The parent polypeptide may comprise a native sequence constant region, or may comprise a constant region with pre-existing amino acid sequence modifications (such as additions, deletions and/or substitutions).

In the context of an Ig polypeptide, the term “constant region” is well understood in the art, and refers to a C-terminal region of an Ig heavy chain, or an Ig light chain. An Ig heavy chain constant region includes CH1, CH2, and CH3 domains (and CH4 domains, where the heavy chain is a u or an & heavy chain). In a native Ig heavy chain, the CH1, CH2, CH3 (and, if present, CH4) domains begin immediately after (C-terminal to) the heavy chain variable (VH) region, and are each from about 100 amino acids to about 130 amino acids in length. In a native Ig light chain, the constant region begins begin immediately after (C-terminal to) the light chain variable (VL) region, and is about 100 amino acids to 120 amino acids in length.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

By “genetically-encodable” as used in reference to an amino acid sequence of polypeptide, peptide or protein means that the amino acid sequence is composed of amino acid residues that are capable of production by transcription and translation of a nucleic acid encoding the amino acid sequence, where transcription and/or translation may occur in a cell or in a cell-free in vitro transcription/translation system.

The term “control sequences” refers to DNA sequences that facilitate expression of an operably linked coding sequence in a particular expression system, e.g. mammalian cell, bacterial cell, cell-free synthesis, etc. The control sequences that are suitable for prokaryote systems, for example, include a promoter, optionally an operator sequence, and a ribosome binding site. Eukaryotic cell systems may utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate the initiation of translation. Generally, “operably linked” means that the DNA sequences being linked are contiguous, and, in the case of a secretory leader, contiguous and in reading frame. Linking is accomplished by ligation or through amplification reactions. Synthetic oligonucleotide adaptors or linkers may be used for linking sequences in accordance with conventional practice.

The term “expression cassette” as used herein refers to a segment of nucleic acid, usually DNA, that can be inserted into a nucleic acid (e.g., by use of restriction sites compatible with ligation into a construct of interest or by homologous recombination into a construct of interest or into a host cell genome). In general, the nucleic acid segment comprises a polynucleotide that encodes a polypeptide of interest, and the cassette and restriction sites are designed to facilitate insertion of the cassette in the proper reading frame for transcription and translation. Expression cassettes can also comprise elements that facilitate expression of a polynucleotide encoding a polypeptide of interest in a host cell, e.g., a mammalian host cell. These elements may include, but are not limited to: a promoter, a minimal promoter, an enhancer, a response element, a terminator sequence, a polyadenylation sequence, and the like.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

By “reactive partner” is meant a molecule or molecular moiety that specifically reacts with another reactive partner to produce a reaction product. Exemplary reactive partners include a cysteine or serine of a sulfatase motif and Formylglycine Generating Enzyme (FGE), which react to form a reaction product of a converted aldehyde tag containing a formylglycine (FGly) in lieu of cysteine or serine in the motif. Other exemplary reactive partners include an aldehyde of an fGly residue of a converted aldehyde tag (e.g., a reactive aldehyde group) and an “aldehyde-reactive reactive partner”, which comprises an aldehyde-reactive group and a moiety of interest, and which reacts to form a reaction product of a modified aldehyde tagged polypeptide having the moiety of interest conjugated to the modified polypeptide through a modified fGly residue.

“N-terminus” refers to the terminal amino acid residue of a polypeptide having a free amine group, which amine group in non-N-terminus amino acid residues normally forms part of the covalent backbone of the polypeptide.

“C-terminus” refers to the terminal amino acid residue of a polypeptide having a free carboxyl group, which carboxyl group in non-C-terminus amino acid residues normally forms part of the covalent backbone of the polypeptide.

By “internal site” as used in referenced to a polypeptide or an amino acid sequence of a polypeptide means a region of the polypeptide that is not at the N-terminus or at the C-terminus.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides anti-CD25 antibody-maytansine conjugate structures. The disclosure also encompasses methods of production of such conjugates, as well as methods of using the same. Embodiments of each are described in more detail in the sections below.

### Antibody-Drug Conjugates

The present disclosure provides conjugates, e.g., antibody-drug conjugates. By “conjugate” is meant a first moiety (e.g., an antibody) is stably associated with a second moiety (e.g., a drug). For example, a maytansine conjugate includes a maytansine (e.g., a maytansine active agent moiety) stably associated with another moiety (e.g., the antibody). By “stably associated” is meant that a moiety is bound to another moiety or structure under standard conditions. In certain embodiments, the first and second moieties are bound to each other through one or more covalent bonds.

In certain embodiments, the conjugate is a polypeptide conjugate, which includes a polypeptide conjugated to a second moiety. In certain embodiments, the moiety conjugated to the polypeptide can be any of a variety of moieties of interest such as, but not limited to, a detectable label, a drug, a water-soluble polymer, or a moiety for immobilization of the polypeptide to a membrane or a surface. In certain embodiments, the conjugate is a maytansine conjugate, where a polypeptide is conjugated to a maytansine or a maytansine active agent moiety. “Maytansine”, “maytansine moiety”, “maytansine active agent moiety” and “maytansinoid” refer to a maytansine and analogs and derivatives thereof, and pharmaceutically active maytansine moieties and/or portions thereof. A maytansine conjugated to the polypeptide can be any of a variety of maytansinoid moieties such as, but not limited to, maytansine and analogs and derivatives thereof as described herein.

The moiety of interest can be conjugated to the polypeptide at any desired site of the polypeptide. Thus, the present disclosure provides, for example, a modified polypeptide having a moiety conjugated at a site at or near the C-terminus of the polypeptide. Other examples include a modified polypeptide having a moiety conjugated at a position at or near the N-terminus of the polypeptide. Examples also include a modified polypeptide having a moiety conjugated at a position between the C-terminus and the N-terminus of the polypeptide (e.g., at an internal site of the polypeptide). Combinations of the above are also possible where the modified polypeptide is conjugated to two or more moieties.

In certain embodiments, a conjugate of the present disclosure includes a maytansine conjugated to an amino acid reside of a polypeptide at the α-carbon of an amino acid residue. Stated another way, a maytansine conjugate includes a polypeptide where the side chain of one or more amino acid residues in the polypeptide have been modified to be attached to a maytansine (e.g., attached to a maytansine through a linker as described herein). For example, a maytansine conjugate includes a polypeptide where the α-carbon of one or more amino acid residues in the polypeptide has been modified to be attached to a maytansine (e.g., attached to a maytansine through a linker as described herein).

Embodiments of the present disclosure include conjugates where a polypeptide is conjugated to one or more moieties, such as 2 moieties, 3 moieties, 4 moieties, 5 moieties, 6 moieties, 7 moieties, 8 moieties, 9 moieties, or 10 or more moieties. The moieties may be conjugated to the polypeptide at one or more sites in the polypeptide. For example, one or more moieties may be conjugated to a single amino acid residue of the polypeptide. In some cases, one moiety is conjugated to an amino acid residue of the polypeptide. In other embodiments, two moieties may be conjugated to the same amino acid residue of the polypeptide. In other embodiments, a first moiety is conjugated to a first amino acid residue of the polypeptide and a second moiety is conjugated to a second amino acid residue of the polypeptide. Combinations of the above are also possible, for example where a polypeptide is conjugated to a first moiety at a first amino acid residue and conjugated to two other moieties at a second amino acid residue. Other combinations are also possible, such as, but not limited to, a polypeptide conjugated to first and second moieties at a first amino acid residue and conjugated to third and fourth moieties at a second amino acid residue, etc.

The one or more amino acid residues of the polypeptide that are conjugated to the one or more moieties may be naturally occurring amino acids, unnatural amino acids, or combinations thereof. For instance, the conjugate may include a moiety conjugated to a naturally occurring amino acid residue of the polypeptide. In other instances, the conjugate may include a moiety conjugated to an unnatural amino acid residue of the polypeptide. One or more moieties may be conjugated to the polypeptide at a single natural or unnatural amino acid residue as described above. One or more natural or unnatural amino acid residues in the polypeptide may be conjugated to the moiety or moieties as described herein. For example, two (or more) amino acid residues (e.g., natural or unnatural amino acid residues) in the polypeptide may each be conjugated to one or two moieties, such that multiple sites in the polypeptide are modified.

As described herein, a polypeptide may be conjugated to one or more moieties. In certain embodiments, the moiety of interest is a chemical entity, such as a drug or a detectable label. For example, a drug (e.g., maytansine) may be conjugated to the polypeptide, or in other embodiments, a detectable label may be conjugated to the polypeptide. Thus, for instance, embodiments of the present disclosure include, but are not limited to, the following: a conjugate of a polypeptide and a drug; a conjugate of a polypeptide and a detectable label; a conjugate of two or more drugs and a polypeptide; a conjugate of two or more detectable labels and a polypeptide; and the like.

In certain embodiments, the polypeptide and the moiety of interest are conjugated through a coupling moiety. For example, the polypeptide and the moiety of interest may each be bound (e.g., covalently bonded) to the coupling moiety, thus indirectly binding the polypeptide and the moiety of interest (e.g., a drug, such as maytansine) together through the coupling moiety. In some cases, the coupling moiety includes a hydrazinyl-indolyl or a hydrazinyl-pyrrolo-pyridinyl compound, or a derivative of a hydrazinyl-indolyl or a hydrazinyl-pyrrolo-pyridinyl compound. For instance, a general scheme for coupling a moiety of interest (e.g., a maytansine) to a polypeptide through a hydrazinyl-indolyl or a hydrazinyl-pyrrolo-pyridinyl coupling moiety is shown in the general reaction scheme below. Hydrazinyl-indolyl and hydrazinyl-pyrrolo-pyridinyl coupling moiety are also referred to herein as a hydrazino-iso-Pictet-Spengler (HIPS) coupling moiety and an aza-hydrazino-iso-Pictet-Spengler (azaHIPS) coupling moiety, respectively.

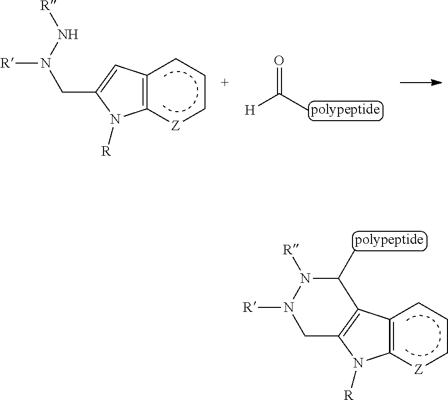

In the reaction scheme above, R is the moiety of interest (e.g., maytansine) that is conjugated to the polypeptide. As shown in the reaction scheme above, a polypeptide that includes a 2-formylglycine residue (fGly) is reacted with a drug (e.g., maytansine) that has been modified to include a coupling moiety (e.g., a hydrazinyl-indolyl or a hydrazinyl-pyrrolo-pyridinyl coupling moiety) to produce a polypeptide conjugate attached to the coupling moiety, thus attaching the maytansine to the polypeptide through the coupling moiety.

As described herein, the moiety can be any of a variety of moieties such as, but not limited to, chemical entity, such as a detectable label, or a drug (e.g., a maytansinoid). R′ and R″ may each independently be any desired substituent, such as, but not limited to, hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. Z may be CR¹¹, NR¹², N, O or S, where R¹¹ and R¹² are each independently selected from any of the substituents described for R′ and R″ above.

Other hydrazinyl-indolyl or hydrazinyl-pyrrolo-pyridinyl coupling moieties are also possible, as shown in the conjugates and compounds described herein. For example, the hydrazinyl-indolyl or hydrazinyl-pyrrolo-pyridinyl coupling moieties may be modified to be attached (e.g., covalently attached) to a linker. As such, embodiments of the present disclosure include a hydrazinyl-indolyl or hydrazinyl-pyrrolo-pyridinyl coupling moiety attached to a drug (e.g., maytansine) through a linker. Various embodiments of the linker that may couple the hydrazinyl-indolyl or hydrazinyl-pyrrolo-pyridinyl coupling moiety to the drug (e.g., maytansine) are described in detail herein.

In certain embodiments, the polypeptide may be conjugated to a moiety of interest, where the polypeptide is modified before conjugation to the moiety of interest. Modification of the polypeptide may produce a modified polypeptide that contains one or more reactive groups suitable for conjugation to the moiety of interest. In some cases, the polypeptide may be modified at one or more amino acid residues to provide one or more reactive groups suitable for conjugation to the moiety of interest (e.g., a moiety that includes a coupling moiety, such as a hydrazinyl-indolyl or a hydrazinyl-pyrrolo-pyridinyl coupling moiety as described above). For example, the polypeptide may be modified to include a reactive aldehyde group (e.g., a reactive aldehyde). A reactive aldehyde may be included in an “aldehyde tag” or “ald-tag”, which as used herein refers to an amino acid sequence derived from a sulfatase motif (e.g., L(C/S)TPSR) that has been converted by action of a formylglycine generating enzyme (FGE) to contain a 2-formylglycine residue (referred to herein as “FGly”). The FGly residue generated by an FGE may also be referred to as a “formylglycine”. Stated differently, the term “aldehyde tag” is used herein to refer to an amino acid sequence that includes a “converted” sulfatase motif (i.e., a sulfatase motif in which a cysteine or serine residue has been converted to FGly by action of an FGE, e.g., L(FGly)TPSR). A converted sulfatase motif may be derived from an amino acid sequence that includes an “unconverted” sulfatase motif (i.e., a sulfatase motif in which the cysteine or serine residue has not been converted to FGly by an FGE, but is capable of being converted, e.g., an unconverted sulfatase motif with the sequence: L(C/S)TPSR). By “conversion” as used in the context of action of a formylglycine generating enzyme (FGE) on a sulfatase motif refers to biochemical modification of a cysteine or serine residue in a sulfatase motif to a formylglycine (FGly) residue (e.g., Cys to FGly, or Ser to FGly). Additional aspects of aldehyde tags and uses thereof in site-specific protein modification are described in U.S. Pat. Nos. 7,985,783 and 8,729,232, the disclosures of each of which are incorporated herein by reference.

In some cases, the modified polypeptide containing the FGly residue may be conjugated to the moiety of interest by reaction of the FGly with a compound (e.g., a compound containing a hydrazinyl-indolyl or a hydrazinyl-pyrrolo-pyridinyl coupling moiety, as described above). For example, an FGly-containing polypeptide may be contacted with a reactive partner-containing drug under conditions suitable to provide for conjugation of the drug to the polypeptide. In some instances, the reactive partner-containing drug may include a hydrazinyl-indolyl or a hydrazinyl-pyrrolo-pyridinyl coupling moiety as described above. For example, a maytansine may be modified to include a hydrazinyl-indolyl or a hydrazinyl-pyrrolo-pyridinyl coupling moiety. In some cases, the maytansine is attached to a hydrazinyl-indolyl or a hydrazinyl-pyrrolo-pyridinyl, such as covalently attached to a a hydrazinyl-indolyl or a hydrazinyl-pyrrolo-pyridinyl through a linker, as described in detail herein.

In certain embodiments, a conjugate of the present disclosure includes a polypeptide (e.g., an antibody, such as an anti-CD25 antibody) having at least one modified amino acid residue. The modified amino acid residue of the polypeptide may be coupled to a drug (e.g., maytansine) containing a hydrazinyl-indolyl or a hydrazinyl-pyrrolo-pyridinyl coupling moiety as described above. In certain embodiments, the modified amino acid residue of the polypeptide (e.g., anti-CD25 antibody) may be derived from a cysteine or serine residue that has been converted to an FGly residue as described above. In certain embodiments, the FGly residue is conjugated to a drug containing a hydrazinyl-indolyl or a hydrazinyl-pyrrolo-pyridinyl coupling moiety as described above to provide a conjugate of the present disclosure where the drug is conjugated to the polypeptide through the hydrazinyl-indolyl or hydrazinyl-pyrrolo-pyridinyl coupling moiety. As used herein, the term FGly′ refers to the modified amino acid residue of the polypeptide (e.g., anti-CD25 antibody) that is coupled to the moiety of interest (e.g., a drug, such as a maytansine).

In certain embodiments, the conjugate includes at least one modified amino acid residue of the formula (I) described herein. For instance, the conjugate may include at least one modified amino acid residue with a side chain of the formula (I):

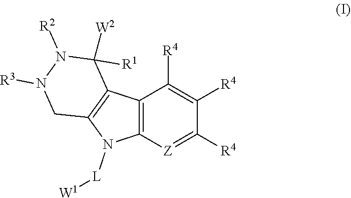

wherein
Z is CR⁴ or N;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is a linker comprising -(T¹-V¹)ₐ-(T²-V²)_{b}-(T³-V³)_{c}-(T⁴-V⁴)_{d}-, wherein a, b, c and d are each independently 0 or 1, where the sum of a, b, c and d is 1 to 4;
T¹, T², T³ and T⁴ are each independently selected from (C₁-C₁₂)alkyl, substituted (C₁-C₁₂)alkyl, (EDA)_{w}, (PEG)ₙ, (AA)ₚ, —(CR¹³OH)ₕ—, piperidin-4-amino (4AP), an acetal group, a hydrazine, a disulfide, and an ester, wherein EDA is an ethylene diamine moiety, PEG is a polyethylene glycol or a modified polyethylene glycol, and AA is an amino acid residue, wherein w is an integer from 1 to 20, n is an integer from 1 to 30, p is an integer from 1 to 20, and h is an integer from 1 to 12;
V¹, V², V³ and V⁴ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
each R¹³ is independently selected from hydrogen, an alkyl, a substituted alkyl, an aryl, and a substituted aryl;
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl;
W¹ is a maytansinoid; and
W² is an anti-CD25 antibody.

In certain embodiments, Z is CR⁴ or N. In certain embodiments, Z is CR⁴. In certain embodiments, Z is N.

In certain embodiments, R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹ is hydrogen. In certain embodiments, R¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹ is methyl. In certain embodiments, R¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is alkynyl or substituted alkynyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl.

In certain embodiments, R² is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R² is hydrogen. In certain embodiments, R² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R² is methyl. In certain embodiments, R² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R² is alkynyl or substituted alkynyl. In certain embodiments, R² is alkoxy or substituted alkoxy. In certain embodiments, R² is amino or substituted amino. In certain embodiments, R² is carboxyl or carboxyl ester. In certain embodiments, R² is acyl or acyloxy. In certain embodiments, R² is acyl amino or amino acyl. In certain embodiments, R² is alkylamide or substituted alkylamide. In certain embodiments, R² is sulfonyl. In certain embodiments, R² is thioalkoxy or substituted thioalkoxy. In certain embodiments, R² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R³ is hydrogen. In certain embodiments, R³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³ is methyl. In certain embodiments, R³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³ is alkynyl or substituted alkynyl. In certain embodiments, R³ is alkoxy or substituted alkoxy. In certain embodiments, R³ is amino or substituted amino. In certain embodiments, R³ is carboxyl or carboxyl ester. In certain embodiments, R³ is acyl or acyloxy. In certain embodiments, R³ is acyl amino or amino acyl. In certain embodiments, R³ is alkylamide or substituted alkylamide. In certain embodiments, R³ is sulfonyl. In certain embodiments, R³ is thioalkoxy or substituted thioalkoxy. In certain embodiments, R³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are optionally cyclically linked to form a 5 or 6-membered heterocyclyl. In certain embodiments, R² and R³ are cyclically linked to form a 5 or 6-membered heterocyclyl. In certain embodiments, R² and R³ are cyclically linked to form a 5-membered heterocyclyl. In certain embodiments, R² and R³ are cyclically linked to form a 6-membered heterocyclyl.

In certain embodiments, each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R⁴ are described in more detail as follows. In certain embodiments, R⁴ is hydrogen. In certain embodiments, each R⁴ is hydrogen. In certain embodiments, R⁴ is halogen, such as F, Cl, Br or I. In certain embodiments, R⁴ is F. In certain embodiments, R⁴ is Cl. In certain embodiments, R⁴ is Br. In certain embodiments, R⁴ is I. In certain embodiments, R⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R⁴ is methyl. In certain embodiments, R⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R⁴ is alkynyl or substituted alkynyl. In certain embodiments, R⁴ is alkoxy or substituted alkoxy. In certain embodiments, R⁴ is amino or substituted amino. In certain embodiments, R⁴ is carboxyl or carboxyl ester. In certain embodiments, R⁴ is acyl or acyloxy. In certain embodiments, R⁴ is acyl amino or amino acyl. In certain embodiments, R⁴ is alkylamide or substituted alkylamide. In certain embodiments, R⁴ is sulfonyl. In certain embodiments, R⁴ is thioalkoxy or substituted thioalkoxy. In certain embodiments, R⁴ is aryl or substituted aryl, such as C₅₋₈ aryl or C₅₋₈ substituted aryl, such as a C₅ aryl or C₅ substituted aryl, or a C₆ aryl or C₆ substituted aryl (e.g., phenyl or substituted phenyl). In certain embodiments, R⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, W¹ is a maytansinoid. Further description of the maytansinoid is found in the disclosure herein.

In certain embodiments, W² is an anti-CD25 antibody. Further description of anti-CD25 antibodies that find use in the subject conjugates is found in the disclosure herein.

In certain embodiments, the compounds of formula (I) include a linker, L. The linker may be utilized to bind a coupling moiety to one or more moieties of interest and/or one or more polypeptides. In some embodiments, the linker binds a coupling moiety to either a polypeptide or a chemical entity. The linker may be bound (e.g., covalently bonded) to the coupling moiety (e.g., as described herein) at any convenient position. For example, the linker may attach a hydrazinyl-indolyl or a hydrazinyl-pyrrolo-pyridinyl coupling moiety to a drug (e.g., a maytansine). The hydrazinyl-indolyl or hydrazinyl-pyrrolo-pyridinyl coupling moiety may be used to conjugate the linker (and thus the drug, e.g., maytansine) to a polypeptide, such as an anti-CD25 antibody. For example, the coupling moiety may be used to conjugate the linker (and thus the drug, e.g., maytansine) to a modified amino acid residue of the polypeptide, such as an FGly reside of an anti-CD25 antibody.

In certain embodiments, L attaches the coupling moiety to W¹, and thus the coupling moiety is indirectly bonded to W¹ through the linker L. As described above, W¹ is a maytansinoid, and thus L attaches the coupling moiety to a maytansinoid, e.g., the coupling moiety is indirectly bonded to the maytansinoid through the linker, L.

Any convenient linkers may be utilized in the subject conjugates and compounds. In certain embodiments, L includes a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, L includes an alkyl or substituted alkyl group. In certain embodiments, L includes an alkenyl or substituted alkenyl group. In certain embodiments, L includes an alkynyl or substituted alkynyl group. In certain embodiments, L includes an alkoxy or substituted alkoxy group. In certain embodiments, L includes an amino or substituted amino group. In certain embodiments, L includes a carboxyl or carboxyl ester group. In certain embodiments, L includes an acyl amino group. In certain embodiments, L includes an alkylamide or substituted alkylamide group. In certain embodiments, L includes an aryl or substituted aryl group. In certain embodiments, L includes a heteroaryl or substituted heteroaryl group. In certain embodiments, L includes a cycloalkyl or substituted cycloalkyl group. In certain embodiments, L includes a heterocyclyl or substituted heterocyclyl group.

In certain embodiments, L includes a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L is a linker described by the formula -(L¹)ₐ-(L²)_{b}-(L³)_{c}-(L⁴)_{d}-, wherein L¹, L², L³ and L⁴ are each independently a linker unit, and a, b, c and d are each independently 0 or 1, wherein the sum of a, b, c and d is 1 to 4.

In certain embodiments, the sum of a, b, c and d is 1. In certain embodiments, the sum of a, b, c and d is 2. In certain embodiments, the sum of a, b, c and d is 3. In certain embodiments, the sum of a, b, c and d is 4. In certain embodiments, a, b, c and d are each 1. In certain embodiments, a, b and c are each 1 and d is 0. In certain embodiments, a and b are each 1 and c and d are each 0. In certain embodiments, a is 1 and b, c and d are each 0.

In certain embodiments, L¹ is attached to the hydrazinyl-indolyl or the hydrazinyl-pyrrolo-pyridinyl coupling moiety (e.g., as shown in formula (I) above). In certain embodiments, L², if present, is attached to W¹. In certain embodiments, L³, if present, is attached to W¹. In certain embodiments, L⁴, if present, is attached to W¹.

Any convenient linker units may be utilized in the subject linkers. Linker units of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³ and L⁴ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L¹ (if present) comprises a polyethylene glycol, a modified polyethylene glycol, an amino acid residue, an alkyl group, a substituted alkyl, an aryl group, a substituted aryl group, or a diamine. In some embodiments, L¹ comprises a polyethylene glycol. In some embodiments, L¹ comprises a modified polyethylene glycol. In some embodiments, L¹ comprises an amino acid residue. In some embodiments, L¹ comprises an alkyl group or a substituted alkyl. In some embodiments, L¹ comprises an aryl group or a substituted aryl group. In some embodiments, L¹ comprises a diamine (e.g., a linking group comprising an alkylene diamine).

In some embodiments, L² (if present) comprises a polyethylene glycol, a modified polyethylene glycol, an amino acid residue, an alkyl group, a substituted alkyl, an aryl group, a substituted aryl group, or a diamine. In some embodiments, L² comprises a polyethylene glycol. In some embodiments, L² comprises a modified polyethylene glycol. In some embodiments, L² comprises an amino acid residue. In some embodiments, L² comprises an alkyl group or a substituted alkyl. In some embodiments, L² comprises an aryl group or a substituted aryl group. In some embodiments, L² comprises a diamine (e.g., a linking group comprising an alkylene diamine).

In some embodiments, L³ (if present) comprises a polyethylene glycol, a modified polyethylene glycol, an amino acid residue, an alkyl group, a substituted alkyl, an aryl group, a substituted aryl group, or a diamine. In some embodiments, L³ comprises a polyethylene glycol. In some embodiments, L³ comprises a modified polyethylene glycol. In some embodiments, L³ comprises an amino acid residue. In some embodiments, L³ comprises an alkyl group or a substituted alkyl. In some embodiments, L³ comprises an aryl group or a substituted aryl group. In some embodiments, L³ comprises a diamine (e.g., a linking group comprising an alkylene diamine).

In some embodiments, L⁴ (if present) comprises a polyethylene glycol, a modified polyethylene glycol, an amino acid residue, an alkyl group, a substituted alkyl, an aryl group, a substituted aryl group, or a diamine. In some embodiments, L⁴ comprises a polyethylene glycol. In some embodiments, L⁴ comprises a modified polyethylene glycol. In some embodiments, L⁴ comprises an amino acid residue. In some embodiments, L⁴ comprises an alkyl group or a substituted alkyl. In some embodiments, L⁴ comprises an aryl group or a substituted aryl group. In some embodiments, L⁴ comprises a diamine (e.g., a linking group comprising an alkylene diamine).

In some embodiments, L is a linker comprising -(L¹)ₐ-(L²)_{b}-(L³)_{c}-(L⁴)_{d}-, where:
-(L¹)ₐ- is -(T¹-V¹)ₐ-;
-(L²)_{b}- is -(T²-V²)_{b}-;
-(L³)_{c}- is -(T³-V³)_{c}-; and
-(L⁴)_{d}- is -(T⁴-V⁴)_{d}-,

wherein T¹, T², T³ and T⁴, if present, are tether groups;

V¹, V², V³ and V⁴, if present, are covalent bonds or linking functional groups; and

a, b, c and d are each independently 0 or 1, wherein the sum of a, b, c and d is 1 to 4.

As described above, in certain embodiments, L¹ is attached to the hydrazinyl-indolyl or the hydrazinyl-pyrrolo-pyridinyl coupling moiety (e.g., as shown in formula (I) above). As such, in certain embodiments, T¹ is attached to the hydrazinyl-indolyl or the hydrazinyl-pyrrolo-pyridinyl coupling moiety (e.g., as shown in formula (I) above). In certain embodiments, V¹ is attached to W¹ (the maytansinoid). In certain embodiments, L², if present, is attached to W¹. As such, in certain embodiments, T², if present, is attached to W¹, or V², if present, is attached to W¹. In certain embodiments, L³, if present, is attached to W¹. As such, in certain embodiments, T³, if present, is attached to W¹, or V³, if present, is attached to W¹. In certain embodiments, L⁴, if present, is attached to W¹. As such, in certain embodiments, T⁴, if present, is attached to W¹, or V⁴, if present, is attached to W¹.

Regarding the tether groups, T¹, T², T³ and T⁴, any convenient tether groups may be utilized in the subject linkers. In some embodiments, T¹, T², T³ and T⁴ each comprise one or more groups independently selected from a (C₁-C₁₂)alkyl, a substituted (C₁-C₁₂)alkyl, an (EDA)_{w}, (PEG)ₙ, (AA)ₚ, —(CR¹³OH)ₕ—, piperidin-4-amino (4AP), an acetal group, a disulfide, a hydrazine, and an ester, where w is an integer from 1 to 20, n is an integer from 1 to 30, p is an integer from 1 to 20, and h is an integer from 1 to 12.

In certain embodiments, when the sum of a, b, c and d is 2 and one of T¹-V¹, T²-V², T³-V³, or T⁴-V⁴ is (PEG)ₕ-CO, then n is not 6. For example, in some instances, the linker may have the following structure:

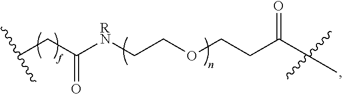

where n is not 6.

In certain embodiments, when the sum of a, b, c and d is 2 and one of T¹-V¹, T²-V², T³-V³, or T⁴-V⁴ is (C₁-C₁₂)alkyl-NR¹⁵, then (C₁-C₁₂)alkyl is not a C₅-alkyl. For example, in some instances, the linker may have the following structure:

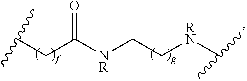

where g is not 4.

In certain embodiments, the tether group (e.g., T¹, T², T³ and/or T⁴) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₂-alkylene.

In certain embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₂-alkylene.

In certain embodiments, the tether group (e.g., T¹, T², T³ and/or T⁴) includes an ethylene diamine (EDA) moiety, e.g., an EDA containing tether. In certain embodiments, (EDA)_{w} includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

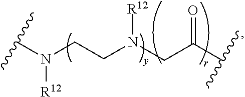

where y is an integer from 1 to 6, r is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group includes a 4-amino-piperidine (4AP) moiety (also referred to herein as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

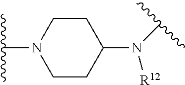

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes a polyethylene glycol moiety described by the formula: (PEG) k, which may be represented by the structure:

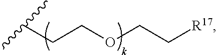

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, COOH, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH.

In certain embodiments, a tether group (e.g., T¹, T², T³ and/or T⁴) includes (PEG)ₙ, where (PEG)ₙ is a polyethylene glycol or a modified polyethylene glycol linking unit. In certain embodiments, (PEG)ₙ is described by the structure:

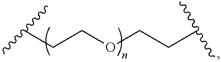

where n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In some instances, n is 2. In some instances, n is 3. In some instances, n is 6. In some instances, n is 12.

In certain embodiments, a tether group (e.g., T¹, T², T³ and/or T⁴) includes (AA)ₚ, where AA is an amino acid residue. Any convenient amino acids may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid, etc. In certain embodiments, p is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In certain embodiments, p is 1. In certain embodiments, p is 2.

In certain embodiments, a tether group (e.g., T¹, T², T³ and/or T⁴) includes a moiety described by the formula —(CR¹³OH)ₕ—, where h is 0 or n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11 or 12. In certain embodiments, h is 1. In certain embodiments, h is 2. In certain embodiments, R¹³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹³ is hydrogen. In certain embodiments, R¹³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹³ is alkynyl or substituted alkynyl. In certain embodiments, R¹³ is alkoxy or substituted alkoxy. In certain embodiments, R¹³ is amino or substituted amino. In certain embodiments, R¹³ is carboxyl or carboxyl ester. In certain embodiments, R¹³ is acyl or acyloxy. In certain embodiments, R¹³ is acyl amino or amino acyl. In certain embodiments, R¹³ is alkylamide or substituted alkylamide. In certain embodiments, R¹³ is sulfonyl. In certain embodiments, R¹³ is thioalkoxy or substituted thioalkoxy. In certain embodiments, R¹³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R¹³ is selected from hydrogen, an alkyl, a substituted alkyl, an aryl, and a substituted aryl. In these embodiments, alkyl, substituted alkyl, aryl, and substituted aryl are as described above for R¹³.

Regarding the linking functional groups, V¹, V², V³ and V⁴, any convenient linking functional groups may be utilized in the subject linkers. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoraidate, and the like. In some embodiments, V¹, V², V³ and V⁴ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵, —NR¹⁵SO₂— and —P(O)OH—, where q is an integer from 1 to 6. In certain embodiments, q is an integer from 1 to 6 (e.g., 1, 2, 3, 4, 5 or 6). In certain embodiments, q is 1. In certain embodiments, q is 2.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R¹⁵ are described in more detail as follows. In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁵ is alkynyl or substituted alkynyl. In certain embodiments, R¹⁵ is alkoxy or substituted alkoxy. In certain embodiments, R¹⁵ is amino or substituted amino. In certain embodiments, R¹⁵ is carboxyl or carboxyl ester. In certain embodiments, R¹⁵ is acyl or acyloxy. In certain embodiments, R¹⁵ is acyl amino or amino acyl. In certain embodiments, R¹⁵ is alkylamide or substituted alkylamide. In certain embodiments, R¹⁵ is sulfonyl. In certain embodiments, R¹⁵ is thioalkoxy or substituted thioalkoxy. In certain embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, the hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl substituents are as described above for R¹⁵.

In certain embodiments, the tether group includes an acetal group, a disulfide, a hydrazine, or an ester. In some embodiments, the tether group includes an acetal group. In some embodiments, the tether group includes a disulfide. In some embodiments, the tether group includes a hydrazine. In some embodiments, the tether group includes an ester.

As described above, in some embodiments, L is a linker comprising -(T¹-V¹)ₐ-(T²-V²)_{b}-(T³-V³)_{c}-(T⁴-V⁴)_{d}-, where a, b, c and d are each independently 0 or 1, where the sum of a, b, c and d is 1 to 4.

In some embodiments, in the subject linker:
T¹ is selected from a (C₁-C₁₂)alkyl and a substituted (C₁-C₁₂)alkyl;
T², T³ and T⁴ are each independently selected from (C₁-C₁₂)alkyl, substituted (C₁-C₁₂)alkyl, (EDA)_{w}, (PEG)ₙ, (AA)ₚ, —(CR¹³OH)ₕ-, 4-amino-piperidine (4AP), an acetal group, a disulfide, a hydrazine, and an ester; and
V¹, V², V³ and V⁴ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
   wherein:
(PEG)ₙ is

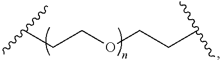

where n is an integer from 1 to 30;
EDA is an ethylene diamine moiety having the following structure:

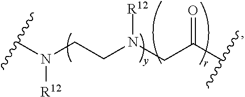

where y is an integer from 1 to 6 and r is 0 or 1;
4-amino-piperidine (4AP) is

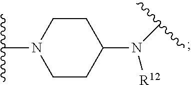

AA is an amino acid residue, where p is an integer from 1 to 20; and
each R¹⁵ and R¹² is independently selected from hydrogen, an alkyl, a substituted alkyl, an aryl and a substituted aryl, wherein any two adjacent R¹² groups may be cyclically linked to form a piperazinyl ring; and
R¹³ is selected from hydrogen, an alkyl, a substituted alkyl, an aryl, and a substituted aryl.

In certain embodiments, T¹, T², T³ and T⁴ and V¹, V², V³ and V⁴ are selected from the following table, e.g., one row of the following table:

TABLE 2

| T¹ | V¹ | T² | V² | T³ | V³ | T⁴ | V⁴ |
|---|---|---|---|---|---|---|---|
| (C₁—C₁₂)alkyl | —CONR¹⁵— | (PEG)ₙ | —CO— | — | — | — | — |
| (C₁—C₁₂)alkyl | —CO— | (AA)ₚ | —NR¹⁵— | (PEG)ₙ | —CO— | — | — |
| (C₁—C₁₂)alkyl | —CO— | (AA)ₚ | — | — | — | — | — |
| (C₁—C₁₂)alkyl | —CONR¹⁵— | (PEG)ₙ | —NR¹⁵— | — | — | — | — |
| (C₁—C₁₂)alkyl | —CO— | (AA)ₚ | —NR¹⁵— | (PEG)ₙ | —NR¹⁵— | — | — |
| (C₁—C₁₂)alkyl | —CO— | (EDA)_{w} | —CO— | — | — | — | — |
| (C₁—C₁₂)alkyl | —CONR¹⁵— | (C₁-C₁₂)alkyl | —NR¹⁵— | — | — | — | — |
| (C₁—C₁₂)alkyl | —CONR¹⁵— | (PEG)ₙ | —CO— | (EDA)_{w} | — | — | — |
| (C₁—C₁₂)alkyl | —CO— | (EDA)_{w} | — | — | — | — | — |
| (C₁—C₁₂)alkyl | —CO— | (EDA)_{w} | —CO— | (CR¹³OH)ₕ | —CONR¹⁵— | (C₁—C₁₂)alkyl | —CO— |
| (C₁—C₁₂)alkyl | —CO— | (AA)ₚ | —NR¹⁵— | (C₁—C₁₂)alkyl | —CO— | — | — |
| (C₁—C₁₂)alkyl | —CONR¹⁵— | (PEG)ₙ | —CO— | (AA)ₚ | — | — | — |
| (C₁—C₁₂)alkyl | —CO— | (EDA)_{w} | —CO— | (CR¹³OH)ₕ | —CO— | (AA)ₚ | — |
| (C₁—C₁₂)alkyl | —CO— | (AA)ₚ | —NR¹⁵— | (C₁—C₁₂)alkyl | —CO— | (AA)ₚ | — |
| (C₁—C₁₂)alkyl | —CO— | (AA)ₚ | —NR¹⁵— | (PEG)ₙ | —CO— | (AA)ₚ | — |
| (C₁—C₁₂)alkyl | —CO— | (AA)ₚ | —NR¹⁵— | (PEG)ₙ | —SO₂— | (AA)ₚ | — |
| (C₁—C₁₂)alkyl | —CO— | (EDA)_{w} | —CO— | (CR¹³OH)ₕ | —CONR¹⁵— | (PEG)ₙ | —CO— |
| (C₁—C₁₂)alkyl | —CO— | (CR¹³OH)ₕ | —CO— | — | — | — | — |
| (C₁—C₁₂)alkyl | —CONR¹⁵— | substituted | —NR¹⁵— | (PEG)ₙ | —CO— | — | — |
| | | (C₁—C₁₂)alkyl | | | | | |
| (C₁—C₁₂)alkyl | —SO₂— | (C₁-C₁₂)alkyl | —CO— | — | — | — | — |
| (C₁—C₁₂)alkyl | —CONR¹⁵— | (C₁-C₁₂)alkyl | — | (CR¹³OH)ₕ | —CONR¹⁵— | — | — |
| (C₁—C₁₂)alkyl | —CO— | (AA)ₚ | —NR¹⁵— | (PEG)ₙ | —CO— | (AA)ₚ | —NR¹⁵— |
| (C₁—C₁₂)alkyl | —CO— | (AA)ₚ | —NR¹⁵— | (PEG)ₙ | —P(O)OH— | (AA)ₚ | — |
| (C₁—C₁₂)alkyl | —CO— | (EDA)_{w} | — | (AA)ₚ | — | — | — |
| (C₁—C₁₂)alkyl | —CONR¹⁵— | (C₁—C₁₂)alkyl | —NR¹⁵— | — | —CO— | — | — |
| (C₁—C₁₂)alkyl | —CONR¹⁵— | (C₁—C₁₂)alkyl | —NR¹⁵— | — | —CO— | (C₁—C₁₂)alkyl | —NR¹⁵— |
| (C₁—C₁₂)alkyl | —CO— | 4AP | —CO— | (C₁—C₁₂)alkyl | —CO— | (AA)ₚ | — |
| (C₁—C₁₂)alkyl | —CO— | 4AP | —CO— | (C₁—C₁₂)alkyl | —CO— | — | — |

In certain embodiments, L is a linker comprising -(L¹)ₐ-(L²)_{b}-(L³)_{c}-(L⁴)_{d}-, where -(L¹)ₐ- is -(T¹-V¹)ₐ-; -(L²)_{b}- is -(T²-V²)_{b}-; -(L³)_{c}- is -(T³-V³)_{c}-; and -(L⁴)_{d}- is -(T⁴-V⁴)_{d}-.

In certain embodiments, T¹ is (C₁-C₁₂)alkyl, V¹ is —CO—, T² is (AA)ₚ, V² is —NR¹⁵—, T³ is (PEG)ₙ, V³ is —CO—, T⁴ is absent and V⁴ is absent.

In certain embodiments, T¹ is (C₁-C₁₂)alkyl, V¹ is —CO—, T² is (EDA)_{w}, V² is —CO—, T³ is (CR¹³OH)ₙ, V³ is —CONR¹⁵—, T⁴ is (C₁-C₁₂)alkyl and V⁴ is —CO—.

In certain embodiments, T¹ is (C₁-C₁₂)alkyl, V¹ is —CO—, T² is (AA)ₚ, V² is —NR¹⁵—, T³ is (C₁-C₁₂)alkyl, V³ is —CO—, T⁴ is absent and V⁴ is absent.

In certain embodiments, T¹ is (C₁-C₁₂)alkyl, V¹ is —CONR¹⁵—, T² is (PEG)ₙ, V² is —CO—, T³ is absent, V³ is absent, T⁴ is absent and V⁴ is absent.

In certain embodiments, T¹ is (C₁-C₁₂)alkyl, V¹ is —CO—, T² is (AA)ₚ, V² is absent, T³ is absent, V³ is absent, T⁴ is absent and V⁴ is absent.

In certain embodiments, T¹ is (C₁-C₁₂)alkyl, V¹ is —CONR¹⁵, T² is (PEG)ₙ, V² is —NR¹⁵—, T³ is absent, V³ is absent, T⁴ is absent and V⁴ is absent.

In certain embodiments, T¹ is (C₁-C₁₂)alkyl, V¹ is —CO—, T² is (AA)ₚ, V² is —NR¹⁵—, T³ is (PEG)ₙ, V³ is —NR¹⁵—, T⁴ is absent and V⁴ is absent.

In certain embodiments, T¹ is (C₁-C₁₂)alkyl, V¹ is —CO—, T² is (EDA)_{w}, V² is —CO—, T³ is absent, V³ is absent, T⁴ is absent and V⁴ is absent.

In certain embodiments, T¹ is (C₁-C₁₂)alkyl, V¹ is —CONR¹⁵—, T² is (C₁-C₁₂)alkyl, V² is —NR¹⁵—, T³ is absent, V³ is absent, T⁴ is absent and V⁴ is absent.

In certain embodiments, T¹ is (C₁-C₁₂)alkyl, V¹ is —CONR¹⁵—, T² is (PEG)ₙ, V² is —CO—, T³ is (EDA)_{w}, V³ is absent, T⁴ is absent and V⁴ is absent.

In certain embodiments, T¹ is (C₁-C₁₂)alkyl, V¹ is —CO—, T² is (EDA)_{w}, V² is absent, T³ is absent, V³ is absent, T⁴ is absent and V⁴ is absent.

In certain embodiments, T¹ is (C₁-C₁₂)alkyl, V¹ is —CONR¹⁵—, T² is (PEG)ₙ, V² is —CO—, T³ is (AA)ₚ, V³ is absent, T⁴ is absent and V⁴ is absent.

In certain embodiments, T¹ is (C₁-C₁₂)alkyl, V¹ is —CO—, T² is (EDA)_{w}, V² is —CO—, T³ is (CR¹³OH)ₙ, V³ is —CO—, T⁴ is (AA)ₚ and V⁴ is absent.

In certain embodiments, T¹ is (C₁-C₁₂)alkyl, V¹ is —CO—, T² is (AA)ₚ, V² is —NR¹⁵—, T³ is (C₁-C₁₂)alkyl, V³ is —CO—, T⁴ is (AA)ₚ and V⁴ is absent.

In certain embodiments, T¹ is (C₁-C₁₂)alkyl, V¹ is —CO—, T² is (AA)ₚ, V² is —NR¹⁵—, T³ is (PEG)ₙ, V³ is —CO—, T⁴ is (AA)ₚ and V⁴ is absent.

In certain embodiments, T¹ is (C₁-C₁₂)alkyl, V¹ is —CO—, T² is (AA)ₚ, V² is —NR¹¹—, T³ is (PEG)ₙ, V³ is —SO₂—, T⁴ is (AA)ₚ and V⁴ is absent.

In certain embodiments, T¹ is (C₁-C₁₂)alkyl, V¹ is —CO—, T² is (EDA)_{w}, V² is —CO—, T³ is (CR¹³OH)ₙ, V³ is —CONR¹⁵—, T⁴ is (PEG)ₙ and V⁴ is —CO—.

In certain embodiments, T¹ is (C₁-C₁₂)alkyl, V¹ is —CO—, T² is (CR¹³OH)ₙ, V² is —CO—, T³ is absent, V³ is absent, T⁴ is absent and V⁴ is absent.

In certain embodiments, T¹ is (C₁-C₁₂)alkyl, V¹ is —CONR¹⁵—, T² is substituted (C₁-C₁₂)alkyl, V² is —NR¹⁵—, T³ is (PEG)ₙ, V³ is —CO—, T⁴ is absent and V⁴ is absent.

In certain embodiments, T¹ is (C₁-C₁₂)alkyl, V¹ is —SO₂—, T² is (C₁-C₁₂)alkyl, V² is —CO—, T³ is absent, V³ is absent, T⁴ is absent and V⁴ is absent.

In certain embodiments, T¹ is (C₁-C₁₂)alkyl, V¹ is —CONR¹⁵—, T² is (C₁-C₁₂)alkyl, V² is absent, T³ is (CR¹³OH)ₙ, V³ is —CONR¹⁵—, T⁴ is absent and V⁴ is absent.

In certain embodiments, T¹ is (C₁-C₁₂)alkyl, V¹ is —CO—, T² is (AA)ₚ, V² is —NR¹⁵—, T³ is (PEG)ₙ, V³ is —CO—, T⁴ is (AA)ₚ and V⁴ is —NR¹⁵—.

In certain embodiments, T¹ is (C₁-C₁₂)alkyl, V¹ is —CO—, T² is (AA)ₚ, V² is —NR¹⁵—, T³ is (PEG)ₙ, V³ is —P(O)OH—, T⁴ is (AA)ₚ and V⁴ is absent.

In certain embodiments, T¹ is (C₁-C₁₂)alkyl, V¹ is —CO—, T² is (EDA)_{w}, V² is absent, T³ is (AA)ₚ, V³ is absent, T⁴ is absent and V⁴ is absent.

In certain embodiments, T¹ is (C₁-C₁₂)alkyl, V¹ is —CO—, T² is (EDA)_{w}, V² is —CO—, T³ is (CR¹³OH)ₙ, V³ is —CONR¹⁵—, T⁴ is (C₁-C₁₂)alkyl and V⁴ is —CO(AA)ₚ-.

In certain embodiments, T¹ is (C₁-C₁₂)alkyl, V¹ is —CONR¹⁵—, T² is (C₁-C₁₂)alkyl, V² is —NR¹⁵—, T³ is absent, V³ is —CO—, T⁴ is absent and V⁴ is absent.

In certain embodiments, T¹ is (C₁-C₁₂)alkyl, V¹ is —CONR¹⁵—, T² is (C₁-C₁₂)alkyl, V² is —NR¹⁵, T³ is absent, V³ is —CO—, T⁴ is (C₁-C₁₂)alkyl and V⁴ is —NR¹⁵.

In certain embodiments, T¹ is (C₁-C₁₂)alkyl, V¹ is —CO—, T² is (EDA)_{w}, V² is —CO—, T³ is (CR¹³OH)ₙ, V³ is —CONR¹⁵—, T⁴ is (PEG)ₙ and V⁴ is —CO(AA)ₚ-.

In certain embodiments, T¹ is (C₁-C₁₂)alkyl, V¹ is —CO—, T² is 4AP, V² is —CO—, T³ is (C₁-C₁₂)alkyl, V³ is —CO—, T⁴ is (AA)ₚ and V⁴ is absent.

In certain embodiments, T¹ is (C₁-C₁₂)alkyl, V¹ is —CO—, T² is 4AP, V² is —CO—, T³ is (C₁-C₁₂)alkyl, V³ is —CO—, T⁴ is absent and V⁴ is absent.

In certain embodiments, the linker is described by one of the following structures:

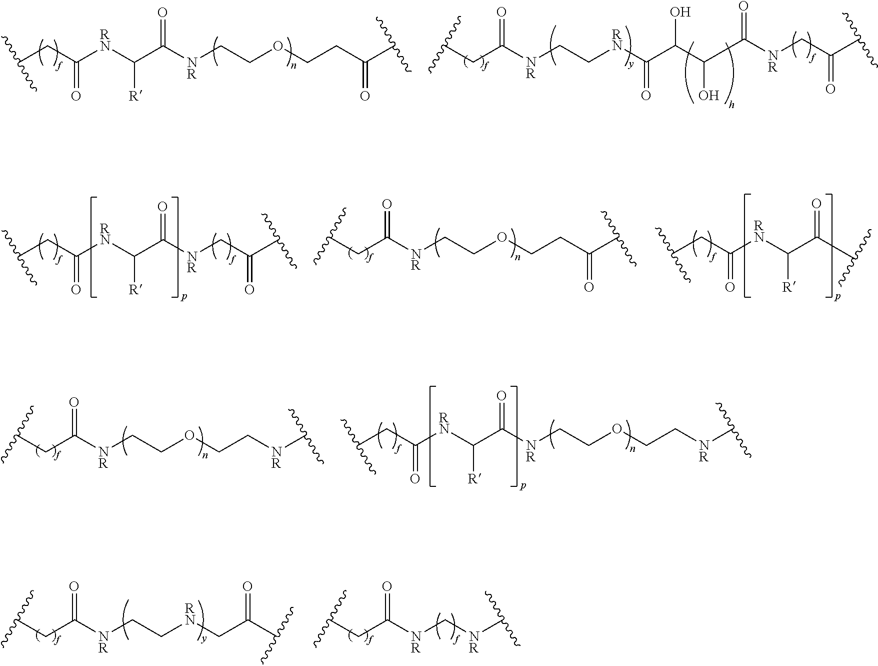

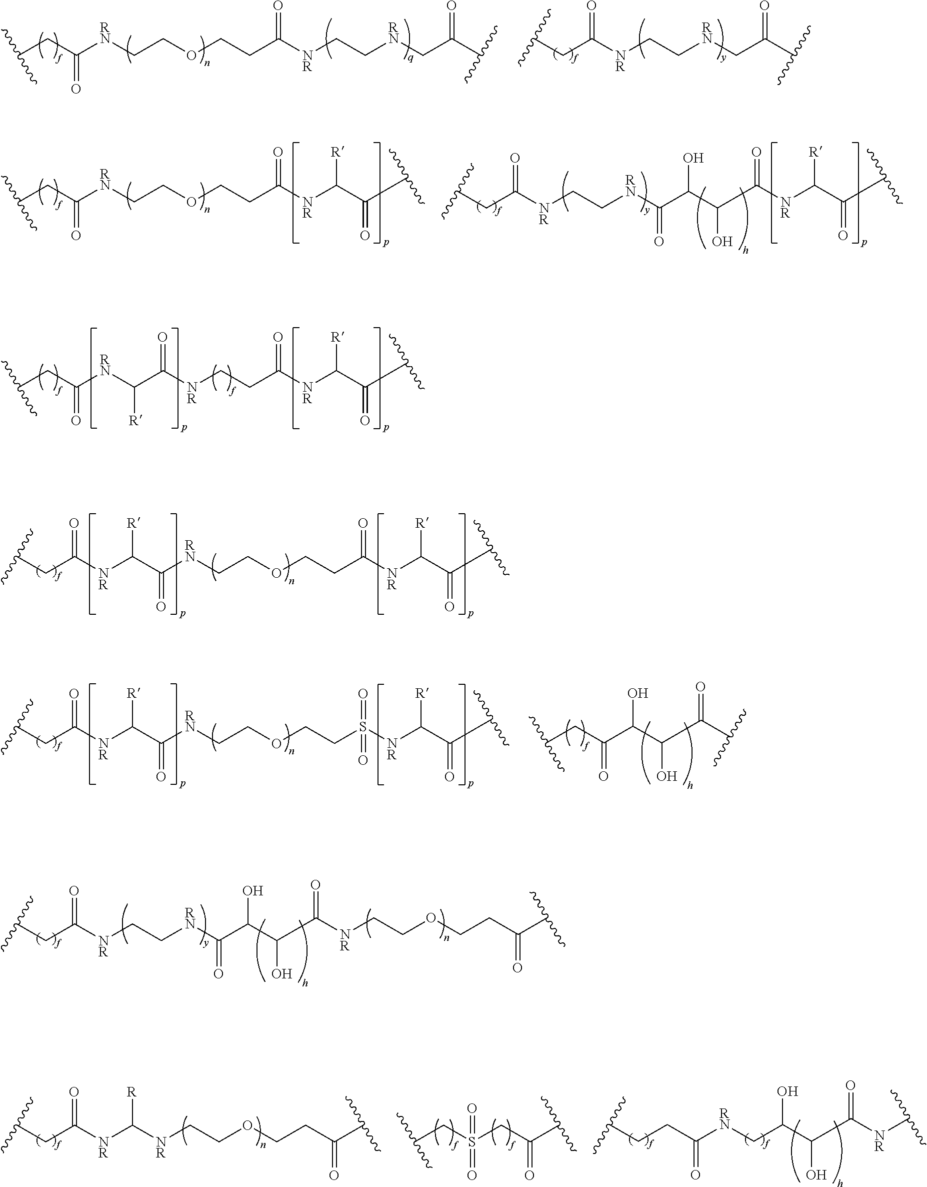

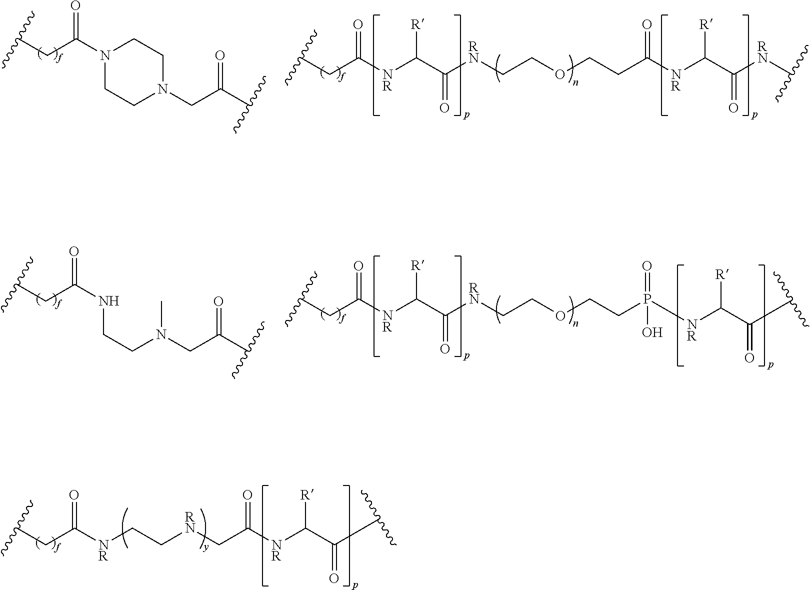

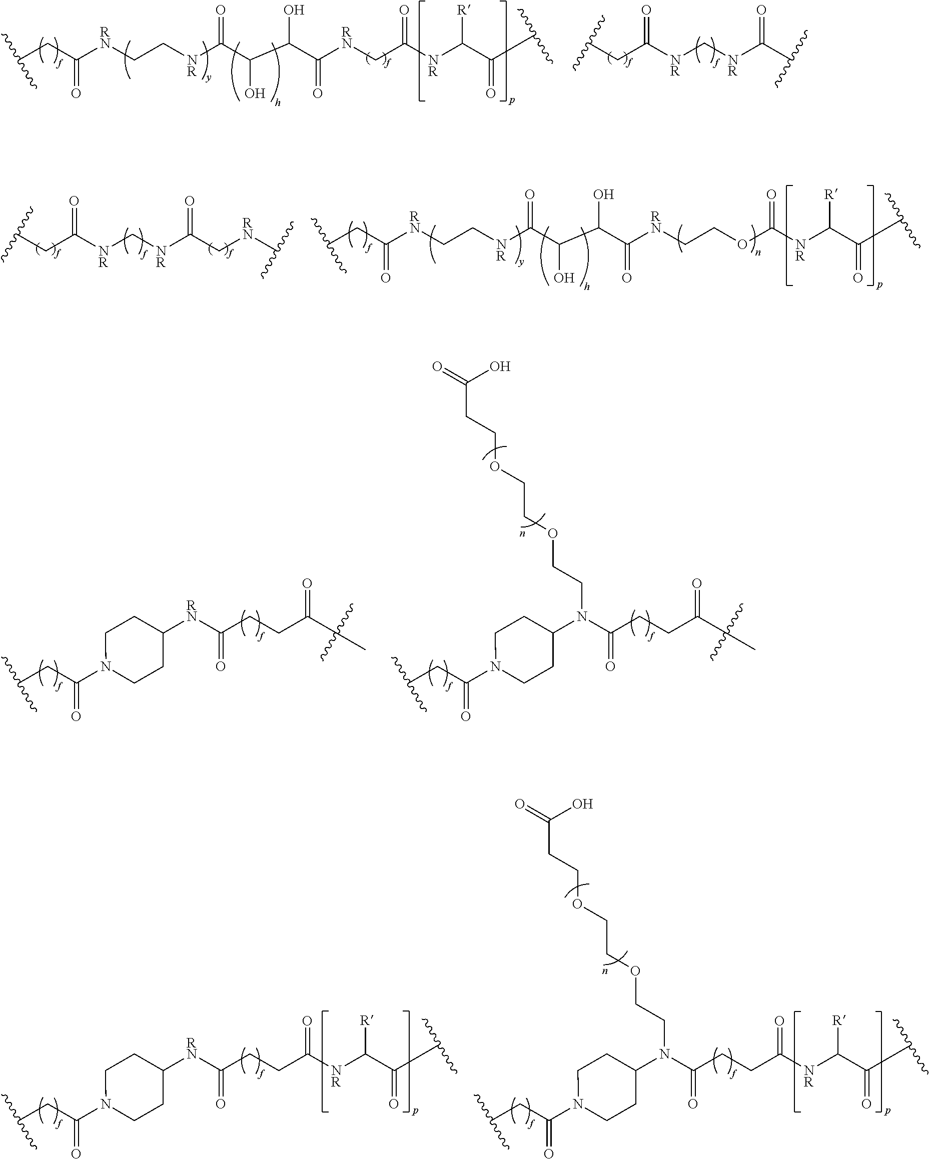

In certain embodiments of the linker structures depicted above, each f is independently 0 or an integer from 1 to 12; each y is independently 0 or an integer from 1 to 20; each n is independently 0 or an integer from 1 to 30; each p is independently 0 or an integer from 1 to 20; each h is independently 0 or an integer from 1 to 12; each R is independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl; and each R′ is independently H, a sidechain of an amino acid, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments of the linker structures depicted above, each f is independently 0, 1, 2, 3, 4, 5 or 6; each y is independently 0, 1, 2, 3, 4, 5 or 6; each n is independently 0, 1, 2, 3, 4, 5 or 6; each p is independently 0, 1, 2, 3, 4, 5 or 6; and each h is independently 0, 1, 2, 3, 4, 5 or 6. In certain embodiments of the linker structures depicted above, each R is independently H, methyl or —(CH₂)ₘ—OH where m is 1, 2, 3 or 4 (e.g., 2).

In certain embodiments of the linker, L, T¹ is (C₁-C₁₂)alkyl, V¹ is —CO—, T² is 4AP, V² is —CO—, T³ is (C₁-C₁₂)alkyl, V³ is —CO—, T⁴ is absent and V⁴ is absent. In certain embodiments, T¹ is ethylene, V¹ is —CO—, T² is 4AP, V² is —CO—, T³ is ethylene, V³ is —CO—, T⁴ is absent and V⁴ is absent. In certain embodiments, T¹ is ethylene, V¹ is —CO—, T² is 4AP, V² is —CO—, T³ is ethylene, V³ is —CO—, T⁴ is absent and V⁴ is absent, where T² (e.g., 4AP) has the following structure:

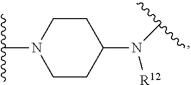

wherein
R¹² is a polyethylene glycol moiety (e.g., a polyethylene glycol or a modified polyethylene glycol).

In certain embodiments, the linker, L, includes the following structure:

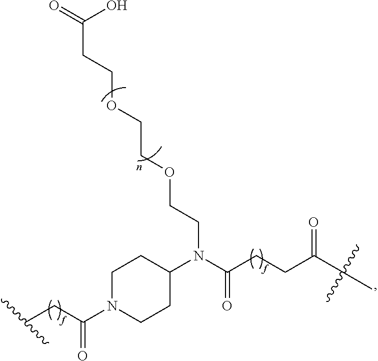

wherein
each f is independently an integer from 1 to 12; and
n is an integer from 1 to 30.

In certain embodiments, f is 1. In certain embodiments, f is 2. In certain embodiments, one f is 2 and one f is 1.

In certain embodiments, n is 1.

In certain embodiments, the left-hand side of the above linker structure is attached to the hydrazinyl-indolyl or the hydrazinyl-pyrrolo-pyridinyl coupling moiety, and the right-hand side of the above linker structure is attached to a maytansine.

Any of the chemical entities, linkers and coupling moieties set forth in the structures above may be adapted for use in the subject compounds and conjugates.

Additional disclosure related to hydrazinyl-indolyl and hydrazinyl-pyrrolo-pyridinyl compounds and methods for producing a conjugate is found in U.S. Application Publication No. 2014/0141025, filed Mar. 11, 2013, and U.S. Application Publication No. 2015/0157736, filed Nov. 26, 2014, the disclosures of each of which are incorporated herein by reference.

### Anti-CD25 Antibodies

As noted above, a subject conjugate can comprise, as substituent W² an anti-CD25 antibody, where the anti-CD25 antibody has been modified to include a 2-formylglycine (FGly) residue. As used herein, amino acids may be referred to by their standard name, their standard three letter abbreviation and/or their standard one letter abbreviation, such as: Alanine or Ala or A; Cysteine or Cys or C; Aspartic acid or Asp or D; Glutamic acid or Glu or E; Phenylalanine or Phe or F; Glycine or Gly or G; Histidine or His or H; Isoleucine or Ile or I; Lysine or Lys or K; Leucine or Leu or L; Methionine or Met or M; Asparagine or Asn or N; Proline or Pro or P; Glutamine or Gln or Q; Arginine or Arg or R; Serine or Ser or S; Threonine or Thr or T; Valine or Val or V; Tryptophan or Trp or W; and Tyrosine or Tyr or Y.

In some cases, a suitable anti-CD25 antibody specifically binds a CD25 polypeptide, where the epitope comprises amino acid residues within a CD25 antigen. In some embodiments, the anti-CD25 antibody binds an epitope within amino acids 101-137, an epitope within amino acids 106-132, an epitope within amino acids 111-127, an epitope comprising amino acids 116-122, or an epitope consisting of amino acids 116-122, of a CD25 amino acid sequence depicted in Table 3 below.

TABLE 3
Human CD25 Amino Acid Sequence

| | |
|---|---|
| Human CD25 Amino | ELCDDDPPEIPHATFKAMAYKEGTMLNCECKRG |
| Acid Sequence | FRRIKSGSLYMLCTGNSSHSSWDNQCQCTSSAT |
| (SEQ ID NO: //) | RNTTKQVTPQPEEQKERKTTEMQSPMQPVDQAS |
| | LPGHCREPPPWENEATERIYHFVVGQMVYYQCV |
| | QGYRALHRGPAESVCKMTHGKTRWTQPQLICTG |
| | EMETSQFPGEEKPQASPEGRPESETSCLVTTTD |
| | FQIQTEMAATMETSIFTTEYQVAVAGCVFLLIS |
| | VLLLSGLTWQRRQRKSRRTI |

The CD25 epitope can be formed by a polypeptide having at least about 75%, at least about 80%, at least about 85%, at least about 90%, at least about 95%, at least about 98%, at least about 99%, or 100%, amino acid sequence identity to a contiguous stretch of from about 200 amino acids to about 251 amino acids of the human CD25 amino acid sequence depicted in Table 3.

A “CD25 antigen” or “CD25 polypeptide” can comprises an amino acid sequence having at least about 75%, at least about 80%, at least about 90%, at least about 95%, at least about 98%, at least about 99%, or 100%, amino acid sequence identity to a contiguous stretch of from about 200 amino acids to about 251 amino acids of the human CD25 amino acid sequence depicted in Table 3.

In some cases, a suitable anti-CD25 antibody exhibits high affinity binding to CD25. For example, in some cases, a suitable anti-CD25 antibody binds to CD25 with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M. In some cases, a suitable anti-CD25 antibody binds to an epitope present on CD25 with an affinity of from about 10⁻⁷ M to about 10⁻⁸ M, from about 10⁻⁸ M to about 10⁻⁹ M, from about 10⁻⁹ M to about 10⁻¹⁰ M, from about 10⁻¹⁰ M to about 10⁻¹¹ M, or from about 10⁻¹¹ M to about 10⁻¹² M, or greater than 10⁻¹² M.

In some cases, a suitable anti-CD25 antibody competes for binding to an epitope within CD25 with a second anti-CD25 antibody (e.g., basiliximab or daclizumab) and/or binds to the same epitope within CD25, as a second anti-CD25 antibody (e.g., basiliximab or daclizumab). In some cases, an anti-CD25 antibody that competes for binding to an epitope within CD25 with a second anti-CD25 antibody also binds to the same epitope as the second anti-CD25 antibody (e.g., basiliximab or daclizumab). In some cases, an anti-CD25 antibody that competes for binding to an epitope within CD25 with a second anti-CD25 antibody binds to an epitope that is overlapping with the epitope bound by the second anti-CD25 antibody (e.g., basiliximab or daclizumab). In some cases, the anti-CD25 antibody is humanized.

In some embodiments, the anti-CD25 antibody is an IgG1 antibody. For example, in certain aspects, the the anti-CD25 antibody is an IgG1 kappa antibody.

In certain aspects, the anti-CD25 antibody competes with interleukin-2 (IL-2) for binding to CD25.

In some embodiments, the anti-CD25 antibody comprises one, two, three, four, five, or all six complementarity determining regions (CDRs) of the anti-CD25 antibody basiliximab. In certain aspects, the anti-CD25 antibody comprises one, two, three, four, five, or all six complementarity determining regions (CDRs) of the anti-CD25 antibody daclizumab.

In certain aspects, the anti-CD25 antibody is a FGly′-containing antibody based on basiliximab or daclizumab. For example, in some embodiments, the antibody is a derivative of daclizumab, where the difference between daclizumab and the derivative is the presence of one or more FGly′ residues (and optionally, the associated FGE recognition sequence amino acids) in the derivative. Provided in Table 4 are nucleic acid and amino acid sequences for an example daclizumab-based antibody according to one embodiment. In the amino acid sequences in Table 4, variable regions are underlined and CDRs are shown in bold. In this example daclizumab-based antibody, the italicized residues at the C-terminus of the heavy chain replace a lysine residue at the C-terminus of a standard IgG1 heavy chain. The underlined residues (LCTPSR) among the italicized residues constitute the aldehyde tag, where the C is converted to an FGly residue by FGE upon expression of the heavy chain. The non-underlined residues among the italicized residues are additional residues that are different from a standard IgG1 heavy chain sequence.

TABLE 4
Example daclizumab-based antibody

| | |
|---|---|
| Light | DIQMTQSPSTLSASVGDRVTITCSASSSISYMHWYQQ |
| chain | KPGKAPKLLIYTTSNLASGVPARFSGSGSGTEFTLTI |
| amino | SSLQPDDFATYYCHQRSTYPLTFGQGTKVEVKRTVAA |
| acid | PSVFIFPPSDEQLKSGTASVVCLLNNFYPREAKVQWK |
| sequence | VDNALQSGNSQESVTEQDSKDSTYSLSSTLTLSKADY |
| (SEQ ID | EKHKVYACEVTHQGLSSPVTKSFNRGEC |
| NO: //) | |
| Heavy | QVQLVQSGAEVKKPGSSVKVSCKASGYTFTSYRMHWV |
| chain | RQAPGQGLEWIGYINPSTGYTEYNQKFKDKATITADE |
| amino | STNTAYMELSSLRSEDTAVYYCARGGGVFDYWGQGTL |
| acid | VTVSSASTKGPSVFPLAPSSKSTSGGTAALGCLVKDY |
| sequence | FPEPVTVSWNSGALTSGVHTFPAVLQSSGLYSLSSVV |
| (SEQ ID | TVPSSSLGTQTYICNVNHKPSNTKVDKRVEPKSCDKT |
| NO: //) | HTCPPCPAPELLGGPSVFLFPPKPKDTLMISRTPEVT |
| | CVVVDVSHEDPEVKFNWYVDGVEVHNAKTKPREEQYN |
| | STYRVVSVLTVLHQDWLNGKEYKCKVSNKALPAPIEK |
| | TISKAKGQPREPQVYTLPPSREEMTKNQVSLTCLVKG |
| | FYPSDIAVEWESNGQPENNYKTTPPVLDSDGSFFLYS |
| | KLTVDKSRWQQGNVFSCSVMHEALHNHYTQKSLSLSP |
| | GSLCTPSRGS |
| Light | GACATCCAGATGACCCAGTCTCCTTCCACCCTGTCTG |
| chain- | CATCTGTAGGAGACAGAGTCACCATCACTTGCAGCGC |
| encoding | CAGCAGCAGTATTAGTTACATGCACTGGTATCAGCAG |
| DNA | AAACCAGGGAAAGCCCCTAAGCTCCTGATCTATACCA |
| sequence | CCTCCAACTTGGCCAGTGGGGTCCCAGCCAGGTTCAG |
| (SEQ ID | CGGCAGTGGATCTGGGACAGAATTCACTCTCACCATC |
| NO: //) | AGCAGCCTGCAGCCTGATGATTTTGCAACTTATTACT |
| | GCCACCAGAGGAGCACCTATCCCCTGACTTTCGGCCA |
| | GGGGACCAAGGTGGAGGTGAAACGTACTGTGGCTGCA |
| | CCATCTGTCTTCATCTTCCCGCCATCTGATGAGCAGT |
| | TGAAATCTGGAACTGCCTCTGTTGTGTGCCTGCTGAA |
| | TAACTTCTATCCCAGAGAGGCCAAAGTACAGTGGAAG |
| | GTGGATAACGCCCTCCAATCGGGTAACTCCCAGGAGA |
| | GTGTCACAGAGCAGGACAGCAAGGACAGCACCTACAG |
| | CCTCAGCAGCACCCTGACGCTGAGCAAAGCAGACTAC |
| | GAGAAACACAAAGTCTACGCCTGCGAAGTCACCCATC |
| | AGGGCCTGAGCTCGCCCGTCACAAAGAGCTTCAACAG |
| | GGGAGAGTGTTAGTGA |
| Heavy | CAGGTGCAGCTGGTGCAGTCTGGGGCTGAGGTGAAGA |
| chain- | AGCCTGGGAGCTCAGTGAAGGTTTCCTGCAAGGCATC |
| encoding | TGGATACACCTTCACCAGCTACAGGATGCACTGGGTG |
| DNA | CGACAGGCCCCTGGACAAGGGCTTGAGTGGATCGGAT |
| sequence | ACATCAACCCTAGTACCGGTTACACAGAGTACAACCA |
| (SEQ ID | GAAGTTCAAGGACAAGGCCACCATCACCGCCGACGAG |
| NO: //) | TCCACGAACACAGCCTACATGGAGCTGAGCAGCCTGA |
| | GATCTGAGGACACGGCCGTGTATTACTGTGCGAGAGG |
| | CGGAGGAGTGTTCGACTACTGGGGCCAGGGAACCCTG |
| | GTGACCGTCTCCTCAGCTAGCACCAAGGGCCCATCGG |
| | TCTTCCCCCTGGCACCCTCCTCCAAGAGCACCTCTGG |
| | GGGCACAGCGGCCCTGGGCTGCCTGGTCAAGGACTAC |
| | TTCCCCGAACCGGTGACGGTGTCGTGGAACTCAGGCG |
| | CCCTGACCAGCGGCGTGCACACCTTCCCGGCTGTCCT |
| | ACAGTCCTCAGGACTCTACTCCCTCAGCAGCGTGGTG |
| | ACCGTGCCCTCCAGCAGCTTGGGCACCCAGACCTACA |
| | TCTGCAACGTGAATCACAAGCCCAGCAACACCAAGGT |
| | GGACAAGAGAGTTGAGCCCAAATCTTGTGACAAAACT |
| | CACACATGCCCACCGTGCCCAGCACCTGAACTCCTGG |
| | GGGGACCGTCAGTCTTCCTCTTCCCCCCAAAACCCAA |
| | GGACACCCTCATGATCTCCCGGACCCCTGAGGTCACA |
| | TGCGTGGTGGTGGACGTGAGCCACGAAGACCCTGAGG |
| | TCAAGTTCAACTGGTACGTGGACGGCGTGGAGGTGCA |
| | TAATGCCAAGACAAAGCCAAGAGAGGAGCAGTACAAC |
| | AGCACGTACCGTGTGGTCAGCGTCCTCACCGTCCTGC |
| | ACCAGGACTGGCTGAATGGCAAGGAGTACAAGTGCAA |
| | GGTCTCCAACAAAGCCCTCCCAGCCCCCATCGAGAAA |
| | ACCATCTCCAAAGCCAAAGGGCAGCCCCGAGAACCAC |
| | AGGTGTACACCCTGCCCCCATCCCGGGAGGAGATGAC |
| | CAAGAACCAGGTCAGCCTGACCTGCCTGGTCAAAGGC |
| | TTCTATCCCAGCGACATCGCCGTGGAGTGGGAGAGCA |
| | ATGGGCAGCCGGAGAACAACTACAAGACCACGCCTCC |
| | CGTGCTGGACTCCGACGGCTCCTTCTTCCTCTACAGC |
| | AAGCTCACCGTGGACAAGAGCAGGTGGCAGCAGGGGA |
| | ACGTCTTCTCATGCTCCGTGATGCATGAGGCTCTGCA |
| | CAACCACTACACGCAGAAGAGCCTCTCCCTGTCTCCC |
| | GGGTCCTTATGTACCCCTTCTAGAGGATCCTGATGA |

In some embodiments, a suitable anti-CD25 antibody can induce apoptosis in a cell that expresses CD25 on its cell surface.

An anti-CD25 antibody suitable for use in a subject conjugate will in some cases inhibit the proliferation of human tumor cells that express on their surface (e.g., overexpress) CD25, where the inhibition occurs in vitro, in vivo, or both in vitro and in vivo. For example, in some cases, an anti-CD25 antibody suitable for use in a subject conjugate inhibits proliferation of human tumor cells that express on their surface (e.g., overexpress) CD25 by at least about 15%, at least about 20%, at least about 25%, at least about 30%, at least about 40%, at least about 50%, at least about 60%, at least about 70%, at least about 80%, or more than 80%, e.g., by at least about 85%, at least about 90%, at least about 95%, at least about 98%, at least about 99%, or 100%.

In some embodiments, the anti-CD25 antibody competes for binding to CD25 (e.g., an epitope of CD25 comprising amino acid residues within a CD25 antigen (e.g., an epitope within amino acids 101-137, an epitope within amino acids 106-132, an epitope within amino acids 111-127, an epitope comprising amino acids 116-122, or an epitope consisting of amino acids 116-122, of a CD25 amino acid sequence depicted in Table 3)) with an antibody comprising a heavy chain complementarity determining region (CDR) selected from SYRMH (daclizumab VH CDR1; SEQ ID NO: //), YINPSTGYTEYNQKFKD (daclizumab VH CDR2; SEQ ID NO: //), GGGVFDY (daclizumab VH CDR3; SEQ ID NO: //), and any combination thereof. In some cases, the anti-CD25antibody is humanized.

In certain aspects, the anti-CD25 antibody competes for binding to CD25 (e.g., an epitope of CD25 comprising amino acid residues within a CD25 antigen (e.g., an epitope within amino acids 101-137, an epitope within amino acids 106-132, an epitope within amino acids 111-127, an epitope comprising amino acids 116-122, or an epitope consisting of amino acids 116-122, of a CD25 amino acid sequence depicted in Table 3)) with an antibody comprising a light-chain CDR selected from SASSSISYMH (daclizumab VL CDR1; SEQ ID NO: //), YTSILHS (daclizumab VL CDR2; SEQ ID NO: //), and QQGNTLPWT (daclizumab VL CDR3; SEQ ID NO: //), and any combination thereof. In some cases, the anti-CD25 antibody is humanized.

In some embodiments, the anti-CD25 antibody competes for binding to CD25 (e.g., an epitope of CD25 comprising amino acid residues within a CD25 antigen (e.g., an epitope within amino acids 101-137, an epitope within amino acids 106-132, an epitope within amino acids 111-127, an epitope comprising amino acids 116-122, or an epitope consisting of amino acids 116-122, of a CD25 amino acid sequence depicted in Table 3)) with an antibody comprising the following six CDRs: SYRMH (daclizumab VH CDR1; SEQ ID NO: //), YINPSTGYTEYNQKFKD (daclizumab VH CDR2; SEQ ID NO: //), GGGVFDY (daclizumab VH CDR3; SEQ ID NO: //), SASSSISYMH (daclizumab VL CDR1; SEQ ID NO: //), YTSILHS (daclizumab VL CDR2; SEQ ID NO: //), and QQGNTLPWT (daclizumab VL CDR3; SEQ ID NO: //). In some cases, the anti-CD25 antibody is humanized.

In some embodiments, the anti-CD25 antibody comprises the following six CDRs: SYRMH (daclizumab VH CDR1; SEQ ID NO: //), YINPSTGYTEYNQKFKD (daclizumab VH CDR2; SEQ ID NO: //), GGGVFDY (daclizumab VH CDR3; SEQ ID NO: //), SASSSISYMH (daclizumab VL CDR1; SEQ ID NO: //), YTSILHS (daclizumab VL CDR2; SEQ ID NO: //), and QQGNTLPWT (daclizumab VL CDR3; SEQ ID NO: //). In some cases, the anti-CD25 antibody is humanized.

In certain aspects, the anti-CD25 antibody comprises VH CDRs present in an anti-CD25 VH region comprising the following amino acid sequence:

(daclizumab VH, SEQ ID NO: //)
QVQLVQSGAEVKKPGSSVKVSCKASGYTFTSYRMHWVRQAPGQGLEWIGYI
NPSTGYTEYNQKFKDKATITADESTNTAYMELSSLRSEDTAVYYCARGGGV
FDYWGQGTLVTVSS.

In some embodiments, the anti-CD25 antibody comprises VL CDRs present in an anti-CD25 VL region comprising the following amino acid sequence:

(daclizumab VL, SEQ ID NO: //)
DIQMTQSPSTLSASVGDRVTITCSASSSISYMHWYQQKPGKAPKLLIYTTS
NLASGVPARFSGSGSGTEFTLTISSLQPDDFATYYCHQRSTYPLTFGQGTK
VEVK.

In certain aspects, the anti-CD25 antibody comprises VH CDRs present in QVQLVQSGAEVKKPGSSVKVSCKASGYTFTSYRMHWVRQAPGQGLEWIGYINPSTGYT EYNQKFKDKATITADESTNTAYMELSSLRSEDTAVYYCARGGGVFDYWGQGTLVTVSS (daclizumab VH, SEQ ID NO: //) and VL CDRs present in DIQMTQSPSTLSASVGDRVTITCSASSSISYMHWYQQKPGKAPKLLIYTTSNLASGVPAR FSGSGSGTEFTLTISSLQPDDFATYYCHQRSTYPLTFGQGTKVEVK (daclizumab VL, SEQ ID NO: //).

### Modified Constant Region Sequences

As noted above, the amino acid sequence of an anti-CD25 antibody is modified to include a sulfatase motif that contains a serine or cysteine residue that is capable of being converted (oxidized) to a 2-formylglycine (FGly) residue by action of a formylglycine generating enzyme (FGE) either in vivo (e.g., at the time of translation of an aldehyde tag-containing protein in a cell) or in vitro (e.g., by contacting an aldehyde tag-containing protein with an FGE in a cell-free system). Such sulfatase motifs may also be referred to herein as an FGE-modification site.

### Sulfatase Motifs

A minimal sulfatase motif of an aldehyde tag is usually 5 or 6 amino acid residues in length, usually no more than 6 amino acid residues in length. Sulfatase motifs provided in an Ig polypeptide are at least 5 or 6 amino acid residues, and can be, for example, from 5 to 16, 6-16, 5-15, 6-15, 5-14, 6-14, 5-13, 6-13, 5-12, 6-12, 5-11, 6-11, 5-10, 6-10, 5-9, 6-9, 5-8, or 6-8 amino acid residues in length, so as to define a sulfatase motif of less than 16, 15, 14, 13, 12, 11, 10, 9, 8 or 7 amino acid residues in length.

In certain embodiments, polypeptides of interest include those where one or more amino acid residues, such as 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more amino acid residues have been inserted, deleted, substituted (replaced) relative to the native amino acid sequence to provide for a sequence of a sulfatase motif in the polypeptide. In certain embodiments, the polypeptide includes a modification (insertion, addition, deletion, and/or substitution/replacement) of less than 20, 19, 18, 17, 16, 15, 14, 13, 12, 11, 10, 9, 8, 7, 6, 5, 4, 3 or 2 amino acid residues of the amino acid sequence relative to the native amino acid sequence of the polypeptide. Where an amino acid sequence native to the polypeptide (e.g., anti-CD25 antibody) contains one or more residues of the desired sulfatase motif, the total number of modifications of residues can be reduced, e.g., by site-specification modification (insertion, addition, deletion, substitution/replacement) of amino acid residues flanking the native amino acid residues to provide a sequence of the desired sulfatase motif. In certain embodiments, the extent of modification of the native amino acid sequence of the target anti-CD25 polypeptide is minimized, so as to minimize the number of amino acid residues that are inserted, deleted, substituted (replaced), or added (e.g., to the N- or C-terminus). Minimizing the extent of amino acid sequence modification of the target anti-CD25 polypeptide may minimize the impact such modifications may have upon anti-CD25 function and/or structure.

It should be noted that while aldehyde tags of particular interest are those comprising at least a minimal sulfatase motif (also referred to a “consensus sulfatase motif”), it will be readily appreciated that longer aldehyde tags are both contemplated and encompassed by the present disclosure and can find use in the compositions and methods of the present disclosure. Aldehyde tags can thus comprise a minimal sulfatase motif of 5 or 6 residues, or can be longer and comprise a minimal sulfatase motif which can be flanked at the N- and/or C-terminal sides of the motif by additional amino acid residues. Aldehyde tags of, for example, 5 or 6 amino acid residues are contemplated, as well as longer amino acid sequences of more than 5, 6, 7, 8, 9, 10, 11, 12, 13, 14, 15, 16, 17, 18, 19, 20 or more amino acid residues.

An aldehyde tag can be present at or near the C-terminus of an Ig heavy chain; e.g., an aldehyde tag can be present within 1, 2, 3, 4, 5, 6, 7, 8, 9, or 10 amino acids of the C-terminus of a native, wild-type Ig heavy chain. An aldehyde tag can be present within a CH1 domain of an Ig heavy chain. An aldehyde tag can be present within a CH2 domain of an Ig heavy chain. An aldehyde tag can be present within a CH3 domain of an Ig heavy chain. An aldehyde tag can be present in an Ig light chain constant region, e.g., in a kappa light chain constant region or a lambda light chain constant region.

In certain embodiments, the sulfatase motif used may be described by the formula:

X¹Z¹⁰X²²Z²⁰X³Z³⁰ (I)
where
Z¹⁰ is cysteine or serine (which can also be represented by (C/S));
Z²⁰ is either a proline or alanine residue (which can also be represented by (P/A));
Z³⁰ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I;
X¹ is present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M, S or V, with the proviso that when the sulfatase motif is at the N-terminus of the target polypeptide, X¹ is present; and

X² and X³ independently can be any amino acid, though usually an aliphatic amino acid, a polar, uncharged amino acid, or a sulfur containing amino acid (i.e., other than an aromatic amino acid or a charged amino acid), e.g., S, T, A, V, G or C, e.g., S, T, A, V or G.

The amino acid sequence of an anti-CD25 heavy and/or light chain can be modified to provide a sequence of at least 5 amino acids of the formula X¹Z¹⁰X²Z²⁰X³Z³⁰, where
Z¹⁰ is cysteine or serine;
Z²⁰ is a proline or alanine residue;
Z³⁰ is an aliphatic amino acid or a basic amino acid;
X¹ is present or absent and, when present, is any amino acid, with the proviso that when the heterologous sulfatase motif is at an N-terminus of the polypeptide, X¹ is present;
X² and X³ are each independently any amino acid,
where the sequence is within or adjacent a solvent-accessible loop region of the Ig constant region, and wherein the sequence is not at the C-terminus of the Ig heavy chain.

The sulfatase motif is generally selected so as to be capable of conversion by a selected FGE, e.g., an FGE present in a host cell in which the aldehyde tagged polypeptide is expressed or an FGE which is to be contacted with the aldehyde tagged polypeptide in a cell-free in vitro method.

For example, where the FGE is a eukaryotic FGE (e.g., a mammalian FGE, including a human FGE), the sulfatase motif can be of the formula:

X¹CX²PX³Z³⁰ (I″)
where
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, S or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G, or C, e.g., S, T, A, V or G; and
Z³⁰ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I.

Specific examples of sulfatase motifs include LCTPSR (SEQ ID NO: //), MCTPSR (SEQ ID NO: //), VCTPSR (SEQ ID NO: //), LCSPSR (SEQ ID NO: //), LCAPSR (SEQ ID NO: //), LCVPSR (SEQ ID NO: //), LCGPSR (SEQ ID NO: //), ICTPAR (SEQ ID NO: //), LCTPSK (SEQ ID NO: //), MCTPSK (SEQ ID NO: //), VCTPSK (SEQ ID NO: //), LCSPSK (SEQ ID NO: //), LCAPSK (SEQ ID NO: //), LCVPSK (SEQ ID NO: //), LCGPSK (SEQ ID NO: //), LCTPSA (SEQ ID NO: //), ICTPAA (SEQ ID NO: //), MCTPSA (SEQ ID NO: //), VCTPSA (SEQ ID NO: //), LCSPSA (SEQ ID NO: //), LCAPSA (SEQ ID NO: //), LCVPSA (SEQ ID NO: //), and LCGPSA (SEQ ID NO: //).

### FGly-Containing Sequences

Upon action of FGE on the modified anti-CD25 heavy and/or light chain, the serine or the cysteine in the sulfatase motif is modified to FGly. Thus, the FGly-containing sulfatase motif can be of the formula:

X¹(FGly)X²Z²⁰X³Z³⁰ (I′″)
where
FGly is the formylglycine residue;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

As described above, the modified polypeptide containing the FGly residue may be conjugated to a drug (e.g., a maytansinoid) by reaction of the FGly with the drug (e.g., a drug containing a hydrazinyl-indolyl or a hydrazinyl-pyrrolo-pyridinyl coupling moiety, as described above) to produce an FGly′-containing sulfatase motif. As used herein, the term FGly′ refers to the modified amino acid residue of the sulfatase motif that is coupled to the drug, such as a maytansinoid (e.g., the modified amino acid residue of formula (I)). Thus, the FGly′-containing sulfatase motif can be of the formula:

X¹(FGly′)X²X²⁰X³Z³⁰ (II)

where
FGly′ is the modified amino acid residue of formula (I);
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

In certain embodiments, the modified amino acid residue of formula (I) is positioned at a C-terminus of a heavy chain constant region of the anti-CD25 antibody. In some instances, the heavy chain constant region comprises a sequence of the formula (II):

X¹(FGly′)X²X²⁰X³Z³⁰ (II)

wherein
FGly′ is the modified amino acid residue of formula (I);
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G; and wherein the sequence is C-terminal to the amino acid sequence QKSLSLSPGK, and where the sequence may include 1, 2, 3, 4, 5, or from 5 to 10, amino acids not present in a native, wild-type heavy Ig chain constant region.

In certain embodiments, the heavy chain constant region comprises the sequence SLSLSPGSL(FGly′)TPSRGS at the C-terminus of the Ig heavy chain, e.g., in place of a native SLSLSPGK (SEQ ID NO: //) sequence.

In certain embodiments, the modified amino acid residue of formula (I) is positioned in a light chain constant region of the anti-CD25 antibody. In certain embodiments, the light chain constant region comprises a sequence of the formula (II):

X¹(FGly′)X²X²⁰X³Z³⁰ (II)

wherein
FGly′ is the modified amino acid residue of formula (I);
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G; and wherein the sequence is C-terminal to the amino acid sequence KVDNAL (SEQ ID NO: //) and/or is N-terminal to the amino acid sequence QSGNSQ (SEQ ID NO: //).

In certain embodiments, the light chain constant region comprises the sequence KVDNAL(FGly′)TPSRQSGNSQ (SEQ ID NO: //).

In certain embodiments, the modified amino acid residue of formula (I) is positioned in a heavy chain CH1 region of the anti-CD25 antibody. In certain embodiments, the heavy chain CH1 region comprises a sequence of the formula (II):

X¹(FGly′)X²X²⁰X³Z³⁰ (II)

wherein
FGly′ is the modified amino acid residue of formula (I);
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G; and wherein the sequence is C-terminal to the amino acid sequence SWNSGA (SEQ ID NO: //) and/or is N-terminal to the amino acid sequence GVHTFP (SEQ ID NO: //).

In certain embodiments, the heavy chain CH1 region comprises the sequence SWNSGAL(FGly′)TPSRGVHTFP (SEQ ID NO: //).

### Site of Modification

As noted above, the amino acid sequence of an anti-CD25 antibody is modified to include a sulfatase motif that contains a serine or cysteine residue that is capable of being converted (oxidized) to an FGly residue by action of an FGE either in vivo (e.g., at the time of translation of an aldehyde tag-containing protein in a cell) or in vitro (e.g., by contacting an aldehyde tag-containing protein with an FGE in a cell-free system). The anti-CD25 polypeptides used to generate a conjugate of the present disclosure include at least an Ig constant region, e.g., an Ig heavy chain constant region (e.g., at least a CH1 domain; at least a CH1 and a CH2 domain; a CH1, a CH2, and a CH3 domain; or a CH1, a CH2, a CH3, and a CH4 domain), or an Ig light chain constant region. Such Ig polypeptides are referred to herein as “target Ig polypeptides” or “target anti-CD25 antibodies” or “target anti-CD25 Ig polypeptides.”

The site in an anti-CD25 antibody into which a sulfatase motif is introduced can be any convenient site. As noted above, in some instances, the extent of modification of the native amino acid sequence of the target anti-CD25 polypeptide is minimized, so as to minimize the number of amino acid residues that are inserted, deleted, substituted (replaced), and/or added (e.g., to the N- or C-terminus). Minimizing the extent of amino acid sequence modification of the target anti-CD25 polypeptide may minimize the impact such modifications may have upon anti-CD25 function and/or structure.

An anti-CD25 antibody heavy chain constant region can include Ig constant regions of any heavy chain isotype, non-naturally occurring Ig heavy chain constant regions (including consensus Ig heavy chain constant regions). An Ig constant region can be modified to include an aldehyde tag, where the aldehyde tag is present in or adjacent a solvent-accessible loop region of the Ig constant region. An Ig constant region can be modified by insertion and/or substitution of 1, 2, 3, 4, 5, 6, 7, 8, 9, 10, 11, 12, 13, 14, 15, or 16 amino acids, or more than 16 amino acids, to provide an amino acid sequence of a sulfatase motif as described above.

In some cases, an aldehyde-tagged anti-CD25 antibody comprises an aldehyde-tagged Ig heavy chain constant region (e.g., at least a CH1 domain; at least a CH1 and a CH2 domain; a CH1, a CH2, and a CH3 domain; or a CH1, a CH2, a CH3, and a CH4 domain). The aldehyde-tagged Ig heavy chain constant region can include heavy chain constant region sequences of an IgA, IgM, IgD, IgE, IgG1, IgG2, IgG3, or IgG4 isotype heavy chain or any allotypic variant of same, e.g., human heavy chain constant region sequences or mouse heavy chain constant region sequences, a hybrid heavy chain constant region, a synthetic heavy chain constant region, or a consensus heavy chain constant region sequence, etc., modified to include at least one sulfatase motif that can be modified by an FGE to generate an FGly-modified Ig polypeptide. Allotypic variants of Ig heavy chains are known in the art. Sec, e.g., Jefferis and Lefranc (2009) MAbs 1:4.

In some cases, an aldehyde-tagged anti-CD25 antibody comprises an aldehyde-tagged Ig light chain constant region. The aldehyde-tagged Ig light chain constant region can include constant region sequences of a kappa light chain, a lambda light chain, e.g., human kappa or lambda light chain constant regions, a hybrid light chain constant region, a synthetic light chain constant region, or a consensus light chain constant region sequence, etc., modified to include at least one sulfatase motif that can be modified by an FGE to generate an FGly-modified anti-CD25 antibody polypeptide. Exemplary constant regions include human gamma 1 and gamma 3 regions. With the exception of the sulfatase motif, a modified constant region may have a wild-type amino acid sequence, or it may have an amino acid sequence that is at least 70% identical (e.g., at least 80%, at least 90% or at least 95% identical) to a wild type amino acid sequence.

In some embodiments the sulfatase motif is at a position other than, or in addition to, the C-terminus of the Ig polypeptide heavy chain. As noted above, an isolated aldehyde-tagged anti-CD25 polypeptide can comprise a heavy chain constant region modified to include a sulfatase motif as described above, where the sulfatase motif is in or adjacent a surface-accessible loop region of the anti-CD25 polypeptide heavy chain constant region.

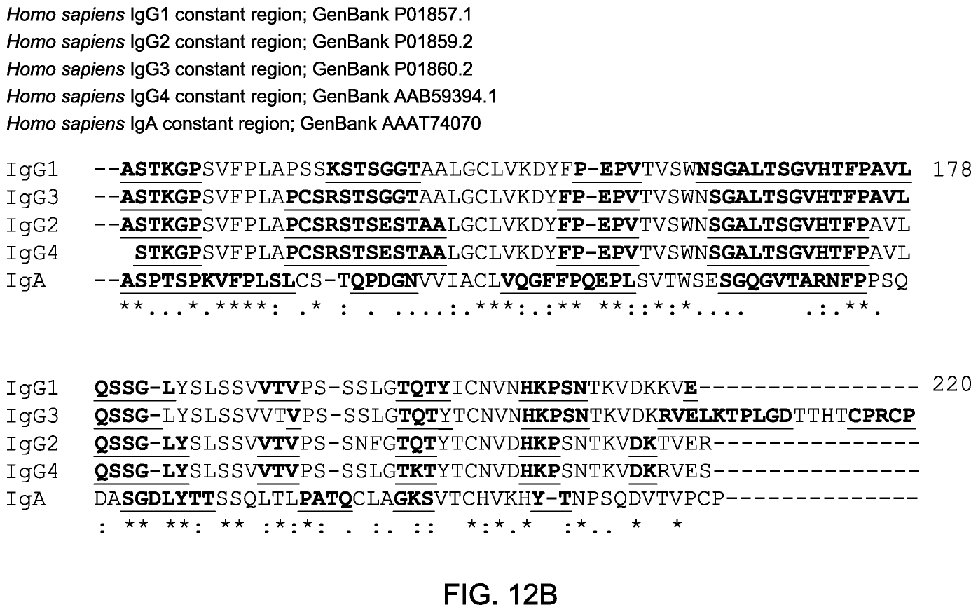

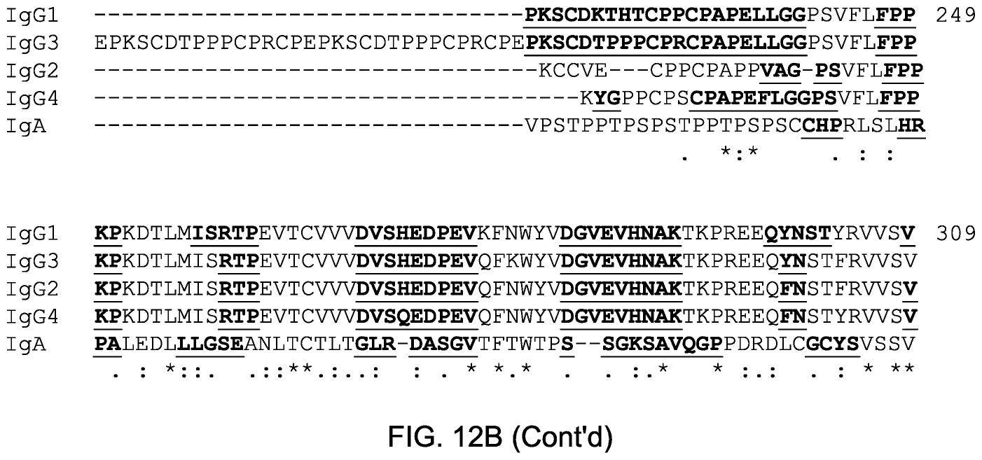

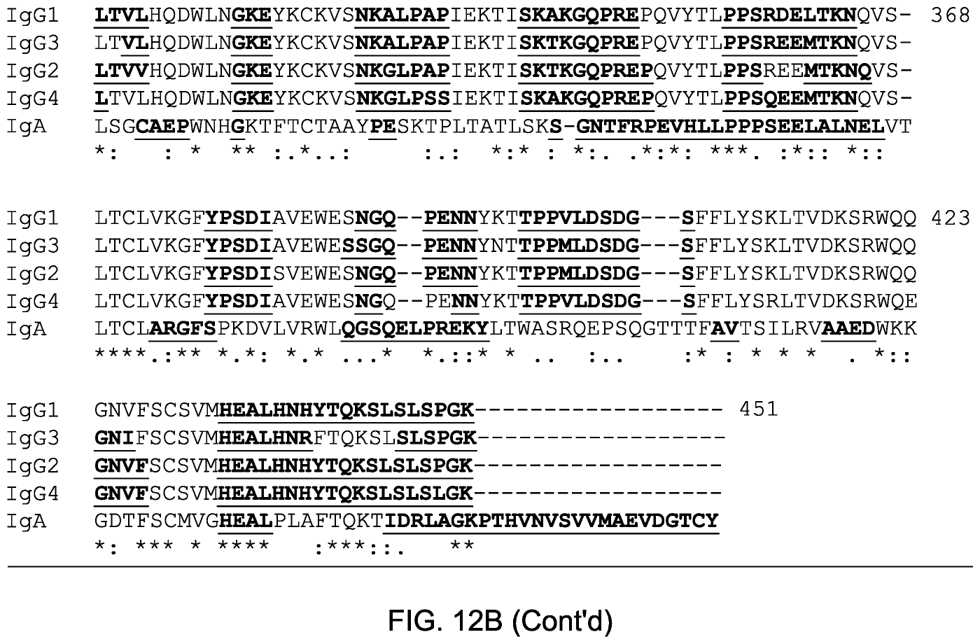

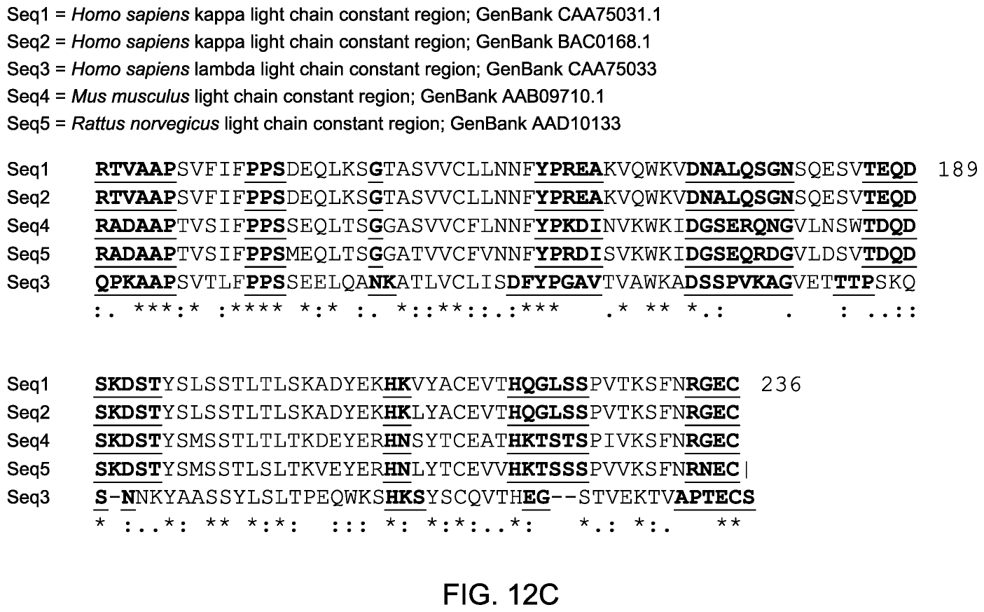

In some instances, a target anti-CD25 immunoglobulin is modified to include a sulfatase motif as described above, where the modification includes one or more amino acid residue insertions, deletions, and/or substitutions. In certain embodiments, the sulfatase motif is within, or adjacent to, a region of an IgG1 heavy chain constant region corresponding to one or more of: 1) amino acids 122-127; 2) amino acids 137-143; 3) amino acids 155-158; 4) amino acids 163-170; 5) amino acids 163-183; 6) amino acids 179-183; 7) amino acids 190-192; 8) amino acids 200-202; 9) amino acids 199-202; 10) amino acids 208-212; 11) amino acids 220-241; 12) amino acids 247-251; 13) amino acids 257-261; 14) amino acid 269-277; 15) amino acids 271-277; 16) amino acids 284-285; 17) amino acids 284-292; 18) amino acids 289-291; 19) amino acids 299-303; 20) amino acids 309-313; 21) amino acids 320-322; 22) amino acids 329-335; 23) amino acids 341-349; 24) amino acids 342-348; 25) amino acids 356-365; 26) amino acids 377-381; 27) amino acids 388-394; 28) amino acids 398-407; 29) amino acids 433-451; and 30) amino acids 446-451; wherein the amino acid numbering is based on the amino acid numbering of human IgG1 as depicted in FIG. 12B.

In some instances, a target anti-CD25 immunoglobulin is modified to include a sulfatase motif as described above, where the modification includes one or more amino acid residue insertions, deletions, and/or substitutions. In certain embodiments, the sulfatase motif is within, or adjacent to, a region of an IgG1 heavy chain constant region corresponding to one or more of: 1) amino acids 1-6; 2) amino acids 16-22; 3) amino acids 34-47; 4) amino acids 42-49; 5) amino acids 42-62; 6) amino acids 34-37; 7) amino acids 69-71; 8) amino acids 79-81; 9) amino acids 78-81; 10) amino acids 87-91; 11) amino acids 100-121; 12) amino acids 127-131; 13) amino acids 137-141; 14) amino acid 149-157; 15) amino acids 151-157; 16) amino acids 164-165; 17) amino acids 164-172; 18) amino acids 169-171; 19) amino acids 179-183; 20) amino acids 189-193; 21) amino acids 200-202; 22) amino acids 209-215; 23) amino acids 221-229; 24) amino acids 22-228; 25) amino acids 236-245; 26) amino acids 217-261; 27) amino acids 268-274; 28) amino acids 278-287; 29) amino acids 313-331; and 30) amino acids 324-331; wherein the amino acid numbering is based on the amino acid numbering of human IgG1 as set out in SEQ ID NO: //(human IgG1 constant region; sequence depicted in FIG. 12B).

Exemplary surface-accessible loop regions of an IgG1 heavy chain include: 1) ASTKGP (SEQ ID NO: //); 2) KSTSGGT (SEQ ID NO: //); 3) PEPV (SEQ ID NO: //); 4) NSGALTSG (SEQ ID NO: //); 5) NSGALTSGVHTFPAVLQSSGL (SEQ ID NO: //); 6) QSSGL (SEQ ID NO: //); 7) VTV; 8) QTY; 9) TQTY (SEQ ID NO: //); 10) HKPSN (SEQ ID NO: //); 11) EPKSCDKTHTCPPCPAPELLGG (SEQ ID NO: //); 12) FPPKP (SEQ ID NO: //); 13) ISRTP (SEQ ID NO: //); 14) DVSHEDPEV (SEQ ID NO: //); 15) SHEDPEV (SEQ ID NO: //); 16) DG; 17) DGVEVHNAK (SEQ ID NO: //); 18) HNA; 19) QYNST (SEQ ID NO: //); 20) VLTVL (SEQ ID NO: //); 21) GKE; 22) NKALPAP (SEQ ID NO: //); 23) SKAKGQPRE (SEQ ID NO: //); 24) KAKGQPR (SEQ ID NO: //); 25) PPSRKELTKN (SEQ ID NO: //); 26) YPSDI (SEQ ID NO: //); 27) NGQPENN (SEQ ID NO: //); 28) TPPVLDSDGS (SEQ ID NO: //); 29) HEALHNHYTQKSLSLSPGK (SEQ ID NO: //); and 30) SLSPGK (SEQ ID NO: //), as shown in FIGS. 12A and 12B.

In some instances, a target immunoglobulin is modified to include a sulfatase motif as described above, where the modification includes one or more amino acid residue insertions, deletions, and/or substitutions. In certain embodiments, the sulfatase motif is within, or adjacent to, a region of an IgG2 heavy chain constant region corresponding to one or more of: 1) amino acids 1-6; 2) amino acids 13-24; 3) amino acids 33-37; 4) amino acids 43-54; 5) amino acids 58-63; 6) amino acids 69-71; 7) amino acids 78-80; 8) 87-89; 9) amino acids 95-96; 10) 114-118; 11) 122-126; 12) 134-136; 13) 144-152; 14) 159-167; 15) 175-176; 16) 184-188; 17) 195-197; 18) 204-210; 19) 216-224; 20) 231-233; 21) 237-241; 22) 252-256; 23) 263-269; 24) 273-282; 25) amino acids 299-302; where the amino acid numbering is based on the numbering of the amino acid sequence set forth in SEQ ID NO: //(human IgG2; also depicted in FIG. 12B).

Exemplary surface-accessible loop regions of an IgG2 heavy chain include 1) ASTKGP (SEQ ID NO: //); 2) PCSRSTSESTAA (SEQ ID NO: //); 3) FPEPV (SEQ ID NO: //); 4) SGALTSGVHTFP (SEQ ID NO: //); 5) QSSGLY (SEQ ID NO: //); 6) VTV; 7) TQT; 8) HKP; 9) DK; 10) VAGPS (SEQ ID NO: //); 11) FPPKP (SEQ ID NO: //); 12) RTP; 13) DVSHEDPEV (SEQ ID NO: //); 14) DGVEVHNAK (SEQ ID NO: //); 15) FN; 16) VLTVV (SEQ ID NO: //); 17) GKE; 18) NKGLPAP (SEQ ID NO: //); 19) SKTKGQPRE (SEQ ID NO: //); 20) PPS; 21) MTKNQ (SEQ ID NO: //); 22) YPSDI (SEQ ID NO: //); 23) NGQPENN (SEQ ID NO: //); 24) TPPMLDSDGS (SEQ ID NO: //); 25) GNVF (SEQ ID NO: //); and 26) HEALHNHYTQKSLSLSPGK (SEQ ID NO: //), as shown in FIG. 12B.

In some instances, a target immunoglobulin is modified to include a sulfatase motif as described above, where the modification includes one or more amino acid residue insertions, deletions, and/or substitutions. In certain embodiments, the sulfatase motif is within, or adjacent to, a region of an IgG3 heavy chain constant region corresponding to one or more of: 1) amino acids 1-6; 2) amino acids 13-22; 3) amino acids 33-37; 4) amino acids 43-61; 5) amino acid 71; 6) amino acids 78-80; 7) 87-91; 8) amino acids 97-106; 9) 111-115; 10) 147-167; 11) 173-177; 16) 185-187; 13) 195-203; 14) 210-218; 15) 226-227; 16) 238-239; 17) 246-248; 18) 255-261; 19) 267-275; 20) 282-291; 21) amino acids 303-307; 22) amino acids 313-320; 23) amino acids 324-333; 24) amino acids 350-352; 25) amino acids 359-365; and 26) amino acids 372-377; where the amino acid numbering is based on the numbering of the amino acid sequence set forth in SEQ ID NO: //(human IgG3; also depicted in FIG. 12B).

Exemplary surface-accessible loop regions of an IgG3 heavy chain include 1) ASTKGP (SEQ ID NO: //); 2) PCSRSTSGGT (SEQ ID NO: //); 3) FPEPV (SEQ ID NO: //); 4) SGALTSGVHTFPAVLQSSG (SEQ ID NO: //); 5) V; 6) TQT; 7) HKPSN (SEQ ID NO: //); 8) RVELKTPLGD (SEQ ID NO: //); 9) CPRCPKP (SEQ ID NO: //); 10) PKSCDTPPPCPRCPAPELLGG (SEQ ID NO: //); 11) FPPKP (SEQ ID NO: //); 12) RTP; 13) DVSHEDPEV (SEQ ID NO: //); 14) DGVEVHNAK (SEQ ID NO: //); 15) YN; 16) VL; 17) GKE; 18) NKALPAP (SEQ ID NO: //); 19) SKTKGQPRE (SEQ ID NO: //); 20) PPSREEMTKN (SEQ ID NO: //); 21) YPSDI (SEQ ID NO: //); 22) SSGQPENN (SEQ ID NO: //); 23) TPPMLDSDGS (SEQ ID NO: //); 24) GNI; 25) HEALHNR (SEQ ID NO: //); and 26) SLSPGK (SEQ ID NO: //), as shown in FIG. 12B.

In some instances, a target immunoglobulin is modified to include a sulfatase motif as described above, where the modification includes one or more amino acid residue insertions, deletions, and/or substitutions. In certain embodiments, the sulfatase motif is within, or adjacent to, a region of an IgG4 heavy chain constant region corresponding to one or more of: 1) amino acids 1-5; 2) amino acids 12-23; 3) amino acids 32-36; 4) amino acids 42-53; 5) amino acids 57-62; 6) amino acids 68-70; 7) amino acids 77-79; 8) amino acids 86-88; 9) amino acids 94-95; 10) amino acids 101-102; 11) amino acids 108-118; 12) amino acids 122-126; 13) amino acids 134-136; 14) amino acids 144-152; 15) amino acids 159-167; 16) amino acids 175-176; 17) amino acids 185-186; 18) amino acids 196-198; 19) amino acids 205-211; 20) amino acids 217-226; 21) amino acids 232-241; 22) amino acids 253-257; 23) amino acids 264-265; 24) 269-270; 25) amino acids 274-283; 26) amino acids 300-303; 27) amino acids 399-417; where the amino acid numbering is based on the numbering of the amino acid sequence set forth in SEQ ID NO: //(human IgG4; also depicted in FIG. 12B).

Exemplary surface-accessible loop regions of an IgG4 heavy chain include 1) STKGP (SEQ ID NO: //); 2) PCSRSTSESTAA (SEQ ID NO: //); 3) FPEPV (SEQ ID NO: //); 4) SGALTSGVHTFP (SEQ ID NO: //); 5) QSSGLY (SEQ ID NO: //); 6) VTV; 7) TKT; 8) HKP; 9) DK; 10) YG; 11) CPAPEFLGGPS (SEQ ID NO: //); 12) FPPKP (SEQ ID NO: //); 13) RTP; 14) DVSQEDPEV (SEQ ID NO: //); 15) DGVEVHNAK (SEQ ID NO: //); 16) FN; 17) VL; 18) GKE; 19) NKGLPSS (SEQ ID NO: //); 20) SKAKGQPREP (SEQ ID NO: //); 21) PPSQEEMTKN (SEQ ID NO: //); 22) YPSDI (SEQ ID NO: //); 23) NG; 24) NN; 25) TPPVLDSDGS (SEQ ID NO: //); 26) GNVF (SEQ ID NO: //); and 27) HEALHNHYTQKSLSLSLGK (SEQ ID NO: //), as shown in FIG. 12B.

In some instances, a target immunoglobulin is modified to include a sulfatase motif as described above, where the modification includes one or more amino acid residue insertions, deletions, and/or substitutions. In certain embodiments, the sulfatase motif is within, or adjacent to, a region of an IgA heavy chain constant region corresponding to one or more of: 1) amino acids 1-13; 2) amino acids 17-21; 3) amino acids 28-32; 4) amino acids 44-54; 5) amino acids 60-66; 6) amino acids 73-76; 7) amino acids 80-82; 8) amino acids 90-91; 9) amino acids 123-125; 10) amino acids 130-133; 11) amino acids 138-142; 12) amino acids 151-158; 13) amino acids 165-174; 14) amino acids 181-184; 15) amino acids 192-195; 16) amino acid 199; 17) amino acids 209-210; 18) amino acids 222-245; 19) amino acids 252-256; 20) amino acids 266-276; 21) amino acids 293-294; 22) amino acids 301-304; 23) amino acids 317-320; 24) amino acids 329-353; where the amino acid numbering is based on the numbering of the amino acid sequence set forth in SEQ ID NO: //(human IgA; also depicted in FIG. 12B).

Exemplary surface-accessible loop regions of an IgA heavy chain include 1) ASPTSPKVFPLSL (SEQ ID NO: //); 2) QPDGN (SEQ ID NO: //); 3) VQGFFPQEPL (SEQ ID NO: //); 4) SGQGVTARNFP (SEQ ID NO: //); 5) SGDLYTT (SEQ ID NO: //); 6) PATQ (SEQ ID NO: //); 7) GKS; 8) YT; 9) CHP; 10) HRPA (SEQ ID NO: //); 11) LLGSE (SEQ ID NO: //); 12) GLRDASGV (SEQ ID NO: //); 13) SSGKSAVQGP (SEQ ID NO: //); 14) GCYS (SEQ ID NO: //); 15) CAEP (SEQ ID NO: //); 16) PE; 17) SGNTFRPEVHLLPPPSEELALNEL (SEQ ID NO: //); 18) ARGFS (SEQ ID NO: //); 19) QGSQELPREKY (SEQ ID NO: //); 20) AV; 21) AAED (SEQ ID NO: //); 22) HEAL (SEQ ID NO: //); and 23) IDRLAGKPTHVNVSVVMAEVDGTCY (SEQ ID NO: //), as shown in FIG. 12B.

A sulfatase motif can be provided within or adjacent one or more of these amino acid sequences of such modification sites of an Ig heavy chain. For example, an Ig heavy chain polypeptide can be modified (e.g., where the modification includes one or more amino acid residue insertions, deletions, and/or substitutions) at one or more of these amino acid sequences to provide a sulfatase motif adjacent and N-terminal and/or adjacent and C-terminal to these modification sites. Alternatively or in addition, an Ig heavy chain polypeptide can be modified (e.g., where the modification includes one or more amino acid residue insertions, deletions, and/or substitutions) at one or more of these amino acid sequences to provide a sulfatase motif between any two residues of the Ig heavy chain modifications sites. In some embodiments, an Ig heavy chain polypeptide may be modified to include two motifs, which may be adjacent to one another, or which may be separated by one, two, three, four or more (e.g., from about 1 to about 25, from about 25 to about 50, or from about 50 to about 100, or more, amino acids. Alternatively or in addition, where a native amino acid sequence provides for one or more amino acid residues of a sulfatase motif sequence, selected amino acid residues of the modification sites of an Ig heavy chain polypeptide amino acid sequence can be modified (e.g., where the modification includes one or more amino acid residue insertions, deletions, and/or substitutions) so as to provide a sulfatase motif at the modification site.

The amino acid sequence of a surface-accessible loop region can thus be modified to provide a sulfatase motif, where the modifications can include insertions, deletions, and/or substitutions. For example, where the modification is in a CH1 domain, the surface-accessible loop region can have the amino acid sequence NSGALTSG (SEQ ID NO: //), and the aldehyde-tagged sequence can be, e.g., NSGALCTPSRG (SEQ ID NO: //), e.g., where the “TS” residues of the NSGALTSG (SEQ ID NO: //) sequence are replaced with “CTPSR,” (SEQ ID NO: //) such that the sulfatase motif has the sequence LCTPSR (SEQ ID NO: //). As another example, where the modification is in a CH2 domain, the surface-accessible loop region can have the amino acid sequence NKALPAP (SEQ ID NO: //), and the aldehyde-tagged sequence can be, e.g., NLCTPSRAP (SEQ ID NO: //), e.g., where the “KAL” residues of the NKALPAP (SEQ ID NO: //) sequence are replaced with “LCTPSR,” (SEQ ID NO: //) such that the sulfatase motif has the sequence LCTPSR (SEQ ID NO: //). As another example, where the modification is in a CH2/CH3 domain, the surface-accessible loop region can have the amino acid sequence KAKGQPR (SEQ ID NO: //), and the aldehyde-tagged sequence can be, e.g., KAKGLCTPSR (SEQ ID NO: //), e.g., where the “GQP” residues of the KAKGQPR (SEQ ID NO: //) sequence are replaced with “LCTPS,” (SEQ ID NO: //) such that the sulfatase motif has the sequence LCTPSR (SEQ ID NO: //).

As noted above, an isolated aldehyde-tagged anti-CD25 Ig polypeptide can comprise a light chain constant region modified to include a sulfatase motif as described above, where the sulfatase motif is in or adjacent a surface-accessible loop region of the Ig polypeptide light chain constant region. Illustrative examples of surface-accessible loop regions of a light chain constant region are presented in FIGS. 12A and 12C.

In some instances, a target immunoglobulin is modified to include a sulfatase motif as described above, where the modification includes one or more amino acid residue insertions, deletions, and/or substitutions. In certain embodiments, the sulfatase motif is within, or adjacent to, a region of an Ig light chain constant region corresponding to one or more of: 1) amino acids 130-135; 2) amino acids 141-143; 3) amino acid 150; 4) amino acids 162-166; 5) amino acids 163-166; 6) amino acids 173-180; 7) amino acids 186-194; 8) amino acids 211-212; 9) amino acids 220-225; 10) amino acids 233-236; wherein the amino acid numbering is based on the amino acid numbering of human kappa light chain as depicted in FIG. 12C. In some instances, a target immunoglobulin is modified to include a sulfatase motif as described above, where the modification includes one or more amino acid residue insertions, deletions, and/or substitutions. In certain embodiments, the sulfatase motif is within, or adjacent to, a region of an Ig light chain constant region corresponding to one or more of: 1) amino acids 1-6; 2) amino acids 12-14; 3) amino acid 21; 4) amino acids 33-37; 5) amino acids 34-37; 6) amino acids 44-51; 7) amino acids 57-65; 8) amino acids 83-83; 9) amino acids 91-96; 10) amino acids 104-107; where the amino acid numbering is based on SEQ ID NO: //(human kappa light chain; amino acid sequence depicted in FIG. 12C).

Exemplary surface-accessible loop regions of an Ig light chain (e.g., a human kappa light chain) include: 1) RTVAAP (SEQ ID NO: //); 2) PPS; 3) Gly (see, e.g., Gly at position 150 of the human kappa light chain sequence depicted in FIG. 12C); 4) YPREA (SEQ ID NO: //); 5) PREA (SEQ ID NO: //); 6) DNALQSGN (SEQ ID NO: //); 7) TEQDSKDST (SEQ ID NO: //); 8) HK; 9) HQGLSS (SEQ ID NO: //); and 10) RGEC (SEQ ID NO: //), as shown in FIGS. 12A and 12C.

Exemplary surface-accessible loop regions of an Ig lambda light chain include QPKAAP (SEQ ID NO: //), PPS, NK, DFYPGAV (SEQ ID NO: //), DSSPVKAG (SEQ ID NO: //), TTP, SN, HKS, EG, and APTECS (SEQ ID NO: //), as shown in FIG. 12C.

In some instances, a target immunoglobulin is modified to include a sulfatase motif as described above, where the modification includes one or more amino acid residue insertions, deletions, and/or substitutions. In certain embodiments, the sulfatase motif is within, or adjacent to, a region of a rat Ig light chain constant region corresponding to one or more of: 1) amino acids 1-6; 2) amino acids 12-14; 3) amino acids 121-22; 4) amino acids 31-37; 5) amino acids 44-51; 6) amino acids 55-57; 7) amino acids 61-62; 8) amino acids 81-83; 9) amino acids 91-92; 10) amino acids 102-105; wherein the amino acid numbering is based on the amino acid numbering of rat light chain as set forth in SEQ ID NO: //(sequence depicted in FIG. 12C).

In some cases, a sulfatase motif is introduced into the CH1 region of an anti-CD25 heavy chain constant region. In some cases, a sulfatase motif is introduced at or near (e.g., within 1 to 10 amino acids of) the C-terminus of an anti-CD25 heavy chain. In some cases, a sulfatase motif is introduced in the light-chain constant region.

In some cases, a sulfatase motif is introduced into the CH1 region of an anti-CD25 heavy chain constant region, e.g., within amino acids 121-219 of the IgG1 heavy chain amino acid sequence depicted in FIG. 12A. For example, in some cases, a sulfatase motif is introduced into the amino acid sequence: ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVE (SEQ ID NO: //). For example, in some of these embodiments, the amino acid sequence GALTSGVH (SEQ ID NO: //) is modified to GALCTPSRGVH (SEQ ID NO: //), where the sulfatase motif is LCTPSR (SEQ ID NO: //).

In some cases, a sulfatase motif is introduced at or near the C-terminus of an anti-CD25 heavy chain, e.g., the sulfatase motifs introduced within 1 amino acid, 2 amino acids (aa), 3 aa, 4 aa, 5 aa, 6 aa, 7 aa, 8 aa, 9 aa, or 10 aa the C-terminus of an anti-CD25 heavy chain. As one non-limiting example, the C-terminal lysine reside of an anti-CD25 heavy chain can be replaced with the amino acid sequence SLCTPSRGS (SEQ ID NO: //).

In some cases, a sulfatase motif is introduced into the constant region of a light chain of an anti-CD25 antibody. As one non-limiting example, in some cases, a sulfatase motif is introduced into the constant region of a light chain of an anti-CD25 antibody, where the sulfatase motif is C-terminal to KVDNAL (SEQ ID NO: //), and/or is N-terminal to QSGNSQ (SEQ ID NO: //). For example, in some cases, the sulfatase motif is LCTPSR (SEQ ID NO: //), and the anti-CD25 light chain comprises the amino acid sequence KVDNALLCTPSRQSGNSQ (SEQ ID NO: //).

### Drugs for Conjugation to a Polypeptide

The present disclosure provides drug-polypeptide conjugates. Examples of drugs include small molecule drugs, such as a cancer chemotherapeutic agent. For example, where the polypeptide is an antibody (or fragment thereof) that has specificity for a tumor cell, the antibody can be modified as described herein to include a modified amino acid, which can be subsequently conjugated to a cancer chemotherapeutic agent, such as a microtubule affecting agents. In certain embodiments, the drug is a microtubule affecting agent that has antiproliferative activity, such as a maytansinoid. In certain embodiments, the drug is a maytansinoid, which as the following structure:

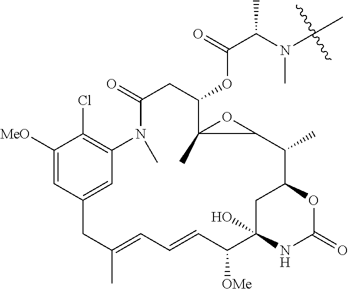

where [symbol] indicates the point of attachment between the maytansinoid and the linker, L, in formula (I). By “point of attachment” is meant that the [symbol] symbol indicates the bond between the N of the maytansinoid and the linker, L, in formula (I). For example, in formula (I), W¹ is a maytansinoid, such as a maytansinoid of the structure above, where [symbol] indicates the point of attachment between the maytansinoid and the linker, L.

As described above, in certain embodiments, L is a linker described by the formula -(L¹)ₐ-(L²)_{b}-(L³)_{c}-(L⁴)_{d}-, wherein L¹, L², L³ and L⁴ are each independently a linker unit. In certain embodiments, L¹ is attached to the coupling moiety, such as a hydrazinyl-indolyl or a hydrazinyl-pyrrolo-pyridinyl coupling moiety (e.g., as shown in formula (I) above). In certain embodiments, L², if present, is attached to W¹ (the maytansinoid). In certain embodiments, L³, if present, is attached to W¹ (the maytansinoid). In certain embodiments, L⁴, if present, is attached to W¹ (the maytansinoid).

As described above, in certain embodiments, the linker -(L¹)ₐ-(L²)_{b}-(L³)_{c}-(L⁴)_{d}- is described by the formula -(T¹-V¹)ₐ-(T²-V²)_{b}-(T³-V³)^{c}-(T⁴-V⁴)_{d}-, wherein a, b, c and d are each independently 0 or 1, where the sum of a, b, c and d is 1 to 4. In certain embodiments, as described above, L¹ is attached to the hydrazinyl-indolyl or the hydrazinyl-pyrrolo-pyridinyl coupling moiety (e.g., as shown in formula (I) above). As such, in certain embodiments, T¹ is attached to the hydrazinyl-indolyl or the hydrazinyl-pyrrolo-pyridinyl coupling moiety (e.g., as shown in formula (I) above). In certain embodiments, V¹ is attached to W¹ (the maytansinoid). In certain embodiments, as described above, L², if present, is attached to W¹ (the maytansinoid). As such, in certain embodiments, T², if present, is attached to W¹ (the maytansinoid), or V², if present, is attached to W¹ (the maytansinoid). In certain embodiments, as described above, L³, if present, is attached to W¹ (the maytansinoid). As such, in certain embodiments, T³, if present, is attached to W¹ (the maytansinoid), or V³, if present, is attached to W¹ (the maytansinoid). In certain embodiments, as described above, L⁴, if present, is attached to W¹ (the maytansinoid). As such, in certain embodiments, T⁴, if present, is attached to W¹ (the maytansinoid), or V⁴, if present, is attached to W¹ (the maytansinoid).

Embodiments of the present disclosure include conjugates where a polypeptide (e.g., anti-CD25 antibody) is conjugated to one or more drug moieties (e.g., maytansinoid), such as 2 drug moieties, 3 drug moieties, 4 drug moieties, 5 drug moieties, 6 drug moieties, 7 drug moieties, 8 drug moieties, 9 drug moieties, or 10 or more drug moieties. The drug moieties may be conjugated to the polypeptide at one or more sites in the polypeptide, as described herein. In certain embodiments, the conjugates have an average drug-to-antibody ratio (DAR) (molar ratio) in the range of from 0.1 to 10, or from 0.5 to 10, or from 1 to 10, such as from 1 to 9, or from 1 to 8, or from 1 to 7, or from 1 to 6, or from 1 to 5, or from 1 to 4, or from 1 to 3, or from 1 to 2. In certain embodiments, the conjugates have an average DAR from 1 to 2, such as 1, 1.1, 1.2, 1.3, 1.4, 1.5, 1.6, 1.7, 1.8, 1.9 or 2. In certain embodiments, the conjugates have an average DAR of 1.5 to 2. In certain embodiments, the conjugates have an average DAR of 1.75 to 1.85. In certain embodiments, the conjugates have an average DAR of 1.8. By average is meant the arithmetic mean.

### Formulations

The conjugates of the present disclosure can be formulated in a variety of different ways. In general, where the conjugate is a polypeptide-drug conjugate, the conjugate is formulated in a manner compatible with the drug conjugated to the polypeptide, the condition to be treated, and the route of administration to be used.

In some embodiments, provided is a pharmaceutical composition that includes any of the conjugates of the present disclosure and a pharmaceutically-acceptable excipient.

The conjugate (e.g., polypeptide-drug conjugate) can be provided in any suitable form, e.g., in the form of a pharmaceutically acceptable salt, and can be formulated for any suitable route of administration, e.g., oral, topical or parenteral administration. Where the conjugate is provided as a liquid injectable (such as in those embodiments where they are administered intravenously or directly into a tissue), the conjugate can be provided as a ready-to-use dosage form, or as a reconstitutable storage-stable powder or liquid composed of pharmaceutically acceptable carriers and excipients.

Methods for formulating conjugates can be adapted from those readily available. For example, conjugates can be provided in a pharmaceutical composition comprising a therapeutically effective amount of a conjugate and a pharmaceutically acceptable carrier (e.g., saline). The pharmaceutical composition may optionally include other additives (e.g., buffers, stabilizers, preservatives, and the like). In some embodiments, the formulations are suitable for administration to a mammal, such as those that are suitable for administration to a human.

### Methods of Treatment

The polypeptide-drug conjugates of the present disclosure find use in treatment of a condition or disease in a subject that is amenable to treatment by administration of the parent drug (i.e., the drug prior to conjugation to the polypeptide).

In some embodiments, provided are methods that include administering to a subject an effective amount of any of the conjugates of the present disclosure.

In certain aspects, provided are methods of delivering a drug to a target site in a subject, the method including administering to the subject a pharmaceutical composition including any of the conjugates of the present disclosure, where the administering is effective to release a therapeutically effective amount of the drug from the conjugate at the target site in the subject.

By “treatment” is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease; and/or (iii) relief, that is, causing the regression of clinical symptoms.

The subject to be treated can be one that is in need of therapy, where the host to be treated is one amenable to treatment using the parent drug. Accordingly, a variety of subjects may be amenable to treatment using the polypeptide-drug conjugates disclosed herein. Generally, such subjects are “mammals”, with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

The amount of polypeptide-drug conjugate administered can be initially determined based on guidance of a dose and/or dosage regimen of the parent drug. In general, the polypeptide-drug conjugates can provide for targeted delivery and/or enhanced serum half-life of the bound drug, thus providing for at least one of reduced dose or reduced administrations in a dosage regimen. Thus, the polypeptide-drug conjugates can provide for reduced dose and/or reduced administration in a dosage regimen relative to the parent drug prior to being conjugated in an polypeptide-drug conjugate of the present disclosure.

Furthermore, as noted above, because the polypeptide-drug conjugates can provide for controlled stoichiometry of drug delivery, dosages of polypeptide-drug conjugates can be calculated based on the number of drug molecules provided on a per polypeptide-drug conjugate basis.

In some embodiments, multiple doses of a polypeptide-drug conjugate are administered. The frequency of administration of a polypeptide-drug conjugate can vary depending on any of a variety of factors, e.g., severity of the symptoms, condition of the subject, etc. For example, in some embodiments, a polypeptide-drug conjugate is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### Methods of Treating Cancer

The present disclosure provides methods that include delivering a conjugate of the present disclosure to an individual having a cancer. The methods are useful for treating a wide variety of cancers, including carcinomas, sarcomas, leukemias, and lymphomas. In the context of cancer, the term “treating” includes one or more (e.g., each) of: reducing growth of a solid tumor, inhibiting replication of cancer cells, reducing overall tumor burden, and ameliorating one or more symptoms associated with a cancer.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's B cell lymphoma; and the like.

In certain aspects, provided are methods of treating cancer in a subject, such methods including administering to the subject a therapeutically effective amount of a pharmaceutical composition including any of the conjugates of the present disclosure, where the administering is effective to treat cancer in the subject. In some embodiments, the cancer is a hematologic malignancy. Hematologic malignancies of interest include, but are not limited to, leukemias, e.g., acute myeloid leukemia (AML). CD25 is up-regulated in refractory AML. In certain aspects, the methods are for treating relapsed and/or refractory AML. In some embodiments, the hematologic malignancy is lymphoma. CD25 is expressed on Hodgkin lymphoma (HL) cells and various T and B cell non-Hodgkin lymphoma (NHL) cells. When the hematologic malignancy is lymphoma, in certain aspects, the lymphoma is Hodgkin lymphoma (HL). When the lymphoma is Hodgkin lymphoma, in certain aspects, the Hodgkin lymphoma is relapsed and/or refractory Hodgkin lymphoma. When the hematologic malignancy is lymphoma, in certain aspects, the lymphoma is Non-Hodgkin lymphoma (NHL). When the lymphoma is Non-Hodgkin lymphoma, in certain aspects, the Non-Hodgkin lymphoma is relapsed and/or refractory Non-Hodgkin lymphoma.

Methods of Reducing Inhibitory Signals Derived from T Regulatory Cells in a Subject

CD25 is expressed on regulatory T cells. As such, in certain aspects, provided are methods of reducing inhibitory signals derived from T regulatory cells in a subject, such methods including administering to the subject a therapeutically effective amount of any of the conjugates of the present disclosure, where the administering is effective to reduce inhibitory signals derived from T regulatory cells in the subject.

The methods of reducing inhibitory signals derived from T regulatory cells find use in a variety of contexts, including but not limited to, as part of an immunomodulatory combination therapy approach by reducing the inhibitory signals derived from the Treg populations and allowing for a productive anti-tumor immune response in a subject having cancer.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. Sec, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4th edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

### Example 1

A linker containing a 4-amino-piperidine (4AP) group was synthesized according to Scheme 1, shown below.

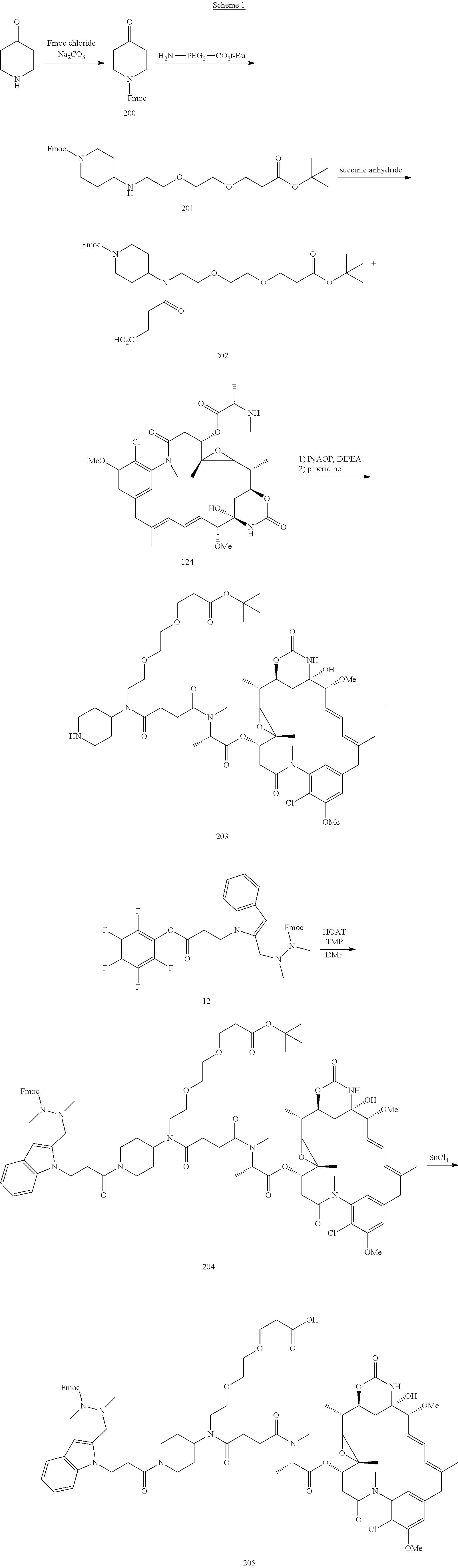

### Synthesis of (9H-fluoren-9-yl)methyl 4-oxopiperidine-1-carboxylate (200)

To a 100 mL round-bottom flask containing a magnetic stir bar was added piperidin-4-one hydrochloride monohydrate (1.53 g, 10 mmol), Fmoc chloride (2.58 g, 10 mmol), sodium carbonate (3.18 g, 30 mmol), dioxane (20 mL), and water (2 mL). The reaction mixture was stirred at room temperature for 1 h. The mixture was diluted with EtOAc (100 mL) and extracted with water (1×100 mL). The organic layer was dried over Na₂SO₄, filtered, and concentrated under reduced pressure. The resulting material was dried in vacuo to yield compound 200 as a white solid (3.05 g, 95% yield).

¹H NMR (CDCl₃) δ 7.78 (d, 2H, J=7.6), 7.59 (d, 2H, J=7.2), 7.43 (t, 2H, J=7.2), 7.37 (t, 2H, J=7.2), 4.60 (d, 2H, J=6.0), 4.28 (t, 2H, J=6.0), 3.72 (br, 2H), 3.63 (br, 2H), 2.39 (br, 2H), 2.28 (br, 2H).

MS (ESI) m/z: [M+H]⁺ Calcd for C₂₀H₂₀NO₃ 322.4; Found 322.2.

### Synthesis of (9H-fluoren-9-yl)methyl 4-((2-(2-(3-(tert-butoxy)-3-oxopropoxy) ethoxy)ethyl)amino) piperidine-1-carboxylate (201)

To a dried scintillation vial containing a magnetic stir bar was added piperidinone 200 (642 mg, 2.0 mmol), H₂N-PEG₂-CO₂t-Bu (560 mg, 2.4 mmol), 4 A molecular sieves (activated powder, 500 mg), and 1,2-dichlorocthane (5 mL). The mixture was stirred for 1 h at room temperature. To the reaction mixture was added sodium triacetoxyborohydride (845 mg, 4.0 mmol). The mixture was stirred for 5 days at room temperature. The resulting mixture was diluted with EtOAc. The organic layer was washed with saturated NaHCO₃ (1×50 mL), and brine (1×50 mL), dried over Na₂SO₄, filtered, and concentrated under reduced pressure to yield compound 201 as an oil, which was carried forward without further purification.

### Synthesis of 13-(1-(((9H-fluoren-9-yl) methoxy) carbonyl) piperidin-4-yl)-2,2-dimethyl-4,14-dioxo-3,7,10-trioxa-13-azaheptadecan-17-oic Acid (202)

To a dried scintillation vial containing a magnetic stir bar was added N-Fmoc-piperidine-4-amino-PEG₂-CO₂t-Bu (201) from the previous step, succinic anhydride (270 mg, 2.7 mmol), and dichloromethane (5 mL). The mixture was stirred for 18 hours at room temperature. The reaction mixture was partitioned between EtOAc and saturated NaHCO₃. The aqueous layer was extracted with EtOAc (3×). The aqueous layer was acidified with HCl (1 M) until the pH ˜3. The aqueous layer was extracted (3×) with DCM. The combined organic layers were dried over Na₂SO₄, filtered, and concentrated under reduced pressure. The reaction mixture was purified by C18 flash chromatography (elute 10-100% MeCN/water with 0.1% acetic acid). Product-containing fractions were concentrated under reduced pressure and then azeotroped with toluene (3×50 mL) to remove residual acetic acid to afford 534 mg (42%, 2 steps) of compound 202 as a white solid.

¹H NMR (DMSO-d₆) δ 11.96 (br, 1H), 7.89 (d, 2H, J=7.2), 7.63 (d, 2H, J=7.2), 7.42 (t, 2H, J=7.2), 7.34 (t, 2H, J=7.2), 4.25-4.55 (m, 3H), 3.70-4.35 (m, 3H), 3.59 (t, 2H, J=6.0), 3.39 (m, 5H), 3.35 (m, 3H), 3.21 (br, 1H), 2.79 (br, 2H), 2.57 (m, 2H), 2.42 (q, 4H, J=6.0), 1.49 (br, 3H), 1.37 (s, 9H).

MS (ESI) m/z: [M+H]⁺ Calcd for C₃₅H₄₇N₂O₉ 639.3; Found 639.2.

### Synthesis of (2S)-1-(((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl)oxy)-2,3-dimethyl-1,4,7-trioxo-8-(piperidin-4-yl)-11,14-dioxa-3,8-diazaheptadecan-17-oic Acid (203)

To a solution of ester 202 (227 mg, 0.356 mmol), diisopropylethylamine (174 μL, 1.065 mmol), N-deacetyl maytansine 124 (231 mg, 0.355 mmol) in 2 mL of DMF was added PyAOP (185 mg, 0.355 mmol). The solution was stirred for 30 min. Piperidine (0.5 mL) was added to the reaction mixture and stirred for an additional 20 min. The crude reaction mixture was purified by C18 reverse phase chromatography using a gradient of 0-100% acetonitrile: water affording 203.2 mg (55%, 2 steps) of compound 203.

### Synthesis of 17-(tert-butyl) 1-((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl) (2S)-8-(1-(3-(2-((2-(((9H-fluoren-9-yl) methoxy) carbonyl)-1,2-dimethylhydrazinyl)methyl)-1H-indol-1-yl) propanoyl) piperidin-4-yl)-2,3-dimethyl-4,7-dioxo-11,14-dioxa-3,8-diazaheptadecanedioate (204)

A solution of piperidine 203 (203.2 mg, 0.194 mmol), ester 12 (126.5 mg, 0.194 mmol), 2,4,6-trimethylpyridine (77 μL, 0.582 mmol), HOAT (26.4 mg, 0.194 mmol) in 1 mL DMF was stirred 30 min. The crude reaction was purified by C18 reverse phase chromatography using a gradient of 0-100% acetonitrile: water with 0.1% formic acid affording 280.5 mg (97% yield) of compound 204.

MS (ESI) m/z: [M+H]⁺ Calcd for C₈₁H₁₀₆ClN₈O₁₈ 1513.7; Found 1514.0.

### Synthesis of (2S)-8-(1-(3-(2-((2-(((9H-fluoren-9-yl) methoxy) carbonyl)-1,2-dimethylhydrazinyl)methyl)-1H-indol-1-yl) propanoyl) piperidin-4-yl)-1-(((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl)oxy)-2,3-dimethyl-1,4,7-trioxo-11,14-dioxa-3,8-diazaheptadecan-17-oic acid (205)

To a solution of compound 204 (108 mg, 0.0714 mmol) in 500 μL anhydrous DCM was added 357 μL of a 1M solution of SnCl₄ in DCM. The heterogeneous mixture was stirred for 1 h and then purified by C18 reverse phase chromatography using a gradient of 0-100% acetonitrile: water with 0.1% formic acid affording 78.4 mg (75% yield) of compound 205.

MS (ESI) m/z: [M−H]⁻ Calcd for C₇₇H₉₆ClN₈O₁₈ 1455.7; Found 1455.9.

### Example 2

A linker containing a 4-amino-piperidine (4AP) group was synthesized according to Scheme 2, shown below.

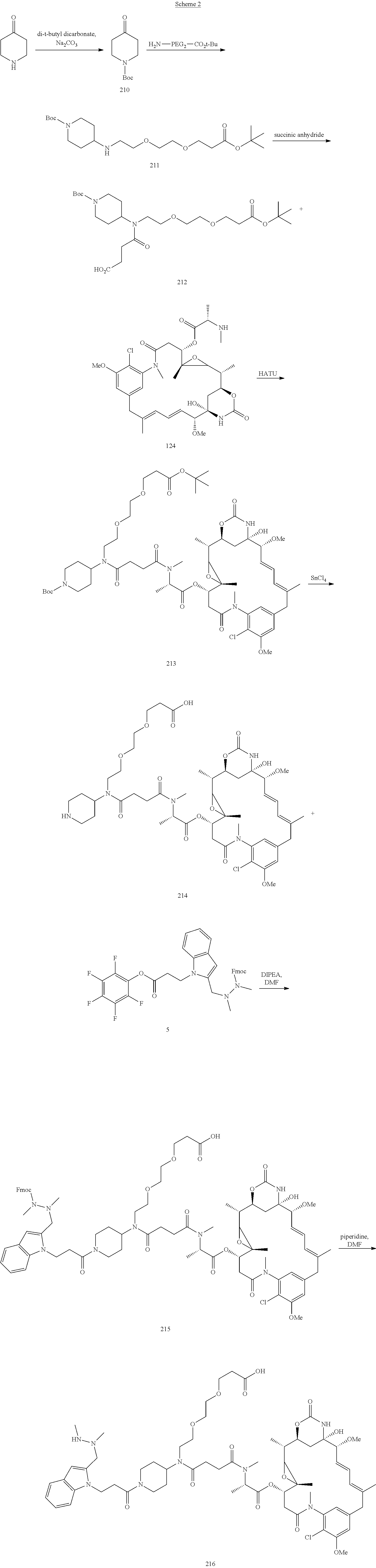

### Synthesis of tert-butyl 4-oxopiperidine-1-carboxylate (210)

To a 100 mL round-bottom flask containing a magnetic stir bar was added piperidin-4-one hydrochloride monohydrate (1.53 g, 10 mmol), di-tert-butyl dicarbonate (2.39 g, 11 mmol), sodium carbonate (1.22 g, 11.5 mmol), dioxane (10 mL), and water (1 mL). The reaction mixture was stirred at room temperature for 1 h. The mixture was diluted with water (100 mL) and extracted with EtOAc (3×100 mL). The combined organic layers were washed with brine, dried over Na₂SO₄, filtered, and concentrated under reduced pressure. The resulting material was dried in vacuo to yield 1.74 g (87%) of compound 210 as a white solid.

¹H NMR (CDCl₃) δ 3.73 (t, 4H, J=6.0), 2.46 (t, 4H, J=6.0), 1.51 (s, 9H).

MS (ESI) m/z: [M+H]⁺ Calcd for C₁₀H₁₈NO₃ 200.3; Found 200.2.

### Synthesis of tert-butyl 4-((2-(2-(3-(tert-butoxy)-3-oxopropoxy) ethoxy)ethyl)amino) piperidine-1-carboxylate (211)

To a dried scintillation vial containing a magnetic stir bar was added tert-butyl 4-oxopiperidine-1-carboxylate (399 mg, 2 mmol), H₂N-PEG₂-COOL-Bu (550 mg, 2.4 mmol), 4 Å molecular sieves (activated powder, 200 mg), and 1,2-dichloroethane (5 mL). The mixture was stirred for 1 h at room temperature. To the reaction mixture was added sodium triacetoxyborohydride (845 mg, 4 mmol). The mixture was stirred for 3 days at room temperature. The resulting mixture was partitioned between EtOAc and saturated aqueous NaHCO₃. The organic layer was washed with brine, dried over Na₂SO₄, filtered, and concentrated under reduced pressure to afford 850 mg of compound 211 as a viscous oil.

MS (ESI) m/z: [M+H]⁺ Calcd for C₂₁H₄₁N₂O₆ 417.3; Found 417.2.

### Synthesis of 13-(1-(tert-butoxycarbonyl) piperidin-4-yl)-2,2-dimethyl-4,14-dioxo-3,7,10-trioxa-13-azaheptadecan-17-oic acid (212)

To a dried scintillation vial containing a magnetic stir bar was added tert-butyl 4-((2-(2-(3-(tert-butoxy)-3-oxopropoxy) ethoxy)ethyl)amino) piperidine-1-carboxylate 211 (220 mg, 0.5 mmol), succinic anhydride (55 mg, 0.55 mmol), 4-(dimethylamino)pyridine (5 mg, 0.04 mmol), and dichloromethane (3 mL). The mixture was stirred for 24 h at room temperature. The reaction mixture was partially purified by flash chromatography (elute 50-100% EtOAc/hexanes) to yield 117 mg of compound 212 as a clear oil, which was carried forward without further characterization.

MS (ESI) m/z: [M+H]⁺ Calcd for C₂₅H₄₅N₂O₉ 517.6; Found 517.5.

### Synthesis of 17-(tert-butyl) 1-((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl) (2S)-8-(1-(tert-butoxycarbonyl) piperidin-4-yl)-2,3-dimethyl-4,7-dioxo-11,14-dioxa-3,8-diazaheptadecanedioate (213)

To a dried scintillation vial containing a magnetic stir bar was added 13-(1-(tert-butoxycarbonyl) piperidin-4-yl)-2,2-dimethyl-4, 14-dioxo-3,7, 10-trioxa-13-azaheptadecan-17-oic acid 212 (55 mg, 0.1 mmol), N-deacyl maytansine 124 (65 mg, 0.1 mmol), HATU (43 mg, 0.11 mmol), DMF (1 mL), and dichloromethane (0.5 mL). The mixture was stirred for 8 h at room temperature. The reaction mixture was directly purified by C18 flash chromatography (elute 5-100% MeCN/water) to give 18 mg (16%) of compound 213 as a white film.

MS (ESI) m/z: [M+H]⁺ Calcd for C₅₇H₈₇ClN₅O₁₇ 1148.6; Found 1148.7.

### Synthesis of (2S)-1-(((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl)oxy)-2,3-dimethyl-1,4,7-trioxo-8-(piperidin-4-yl)-11,14-dioxa-3,8-diazaheptadecan-17-oic acid (214)

To a dried scintillation vial containing a magnetic stir bar was added maytansinoid 213 (31 mg, 0.027 mmol) and dichloromethane (1 mL). The solution was cooled to 0° C. and tin (IV) tetrachloride (1.0 M solution in dichloromethane, 0.3 mL, 0.3 mmol) was added. The reaction mixture was stirred for 1 h at 0° C. The reaction mixture was directly purified by C18 flash chromatography (elute 5-100% MeCN/water) to yield 16 mg (60%) of compound 214 as a white solid (16 mg, 60% yield).

MS (ESI) m/z: [M+H]⁺ Calcd for C₄₈H₇₁ClN₅O₁₅ 992.5; Found 992.6.

### Synthesis of (2S)-8-(1-(3-(2-((2-(((9H-fluoren-9-yl) methoxy) carbonyl)-1,2-dimethylhydrazinyl)methyl)-1H-indol-1-yl) propanoyl) piperidin-4-yl)-1-(((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl)oxy)-2,3-dimethyl-1,4,7-trioxo-11,14-dioxa-3,8-diazaheptadecan-17-oic acid (215)

To a dried scintillation vial containing a magnetic stir bar was added maytansinoid 214 (16 mg, 0.016 mmol), (9H-fluoren-9-yl)methyl 1,2-dimethyl-2-((1-(3-oxo-3-(perfluorophenoxy) propyl)-1H-indol-2-yl)methyl) hydrazine-1-carboxylate (5) (13 mg, 0.02 mmol), DIPEA (8 μL, 0.05 mmol), and DMF (1 mL). The solution was stirred for 18 h at room temperature. The reaction mixture was directly purified by C18 flash chromatography (elute 5-100% MeCN/water) to yield 18 mg (77%) of compound 215 as a white solid.

MS (ESI) m/z: [M+H]⁺ Calcd for C₇₇H₉₈ClN₈O₁₈ 1457.7; Found 1457.9.

### Synthesis of (2S)-1-(((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl)oxy)-8-(1-(3-(2-((1,2-dimethylhydrazinyl)methyl)-1H-indol-1-yl) propanoyl) piperidin-4-yl)-2,3-dimethyl-1,4,7-trioxo-11,14-dioxa-3,8-diazaheptadecan-17-oic acid (216)

To a dried scintillation vial containing a magnetic stir bar was added maytansinoid 215 (18 mg, 0.012 mmol), piperidine (20 μL, 0.02 mmol), and DMF (1 mL). The solution was stirred for 20 minutes at room temperature. The reaction mixture was directly purified by C18 flash chromatography (elute 1-60% MeCN/water) to yield 15 mg (98%) of compound 216 (also referred to herein as HIPS-4AP-maytansine or HIPS-4-amino-piperidin-maytansinc) as a white solid.

MS (ESI) m/z: [M+H]⁺ Calcd for C₆₂H₈₈ClN₈O₁₆ 1235.6; Found 1236.0.

### Example 3

### Experimental Procedures

### General

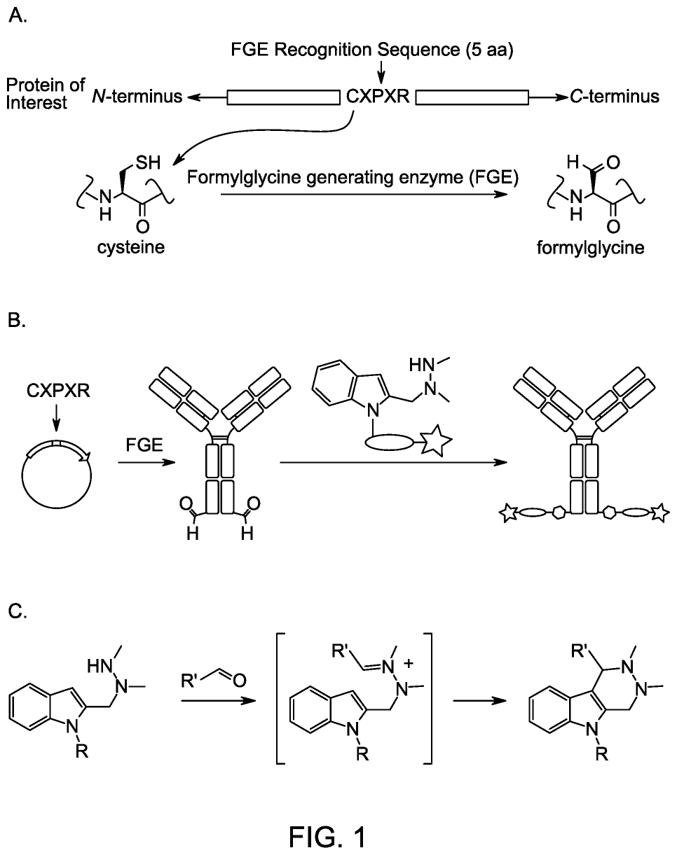

Experiments were performed to create site-specifically conjugated antibody-drug conjugates (ADCs). Site-specific ADC production included the incorporation of formylglycine (FGly), a non-natural amino acid, into the protein sequence. To install FGly (FIG. 1), a short consensus sequence, CXPXR, where X is serine, threonine, alanine, or glycine, was inserted at the desired location in the conserved regions of antibody heavy or light chains using standard molecular biology cloning techniques. This “tagged” construct was produced recombinantly in cells that coexpress the formylglycine-generating enzyme (FGE), which cotranslationally converted the cysteine within the tag into an FGly residue, generating an aldehyde functional group (also referred to herein as an aldehyde tag). The aldehyde functional group served as a chemical handle for bioorthogonal conjugation. A hydrazino-iso-Pictet-Spengler (HIPS) ligation was used to connect the payload (e.g., a drug, such as a cytotoxin (e.g., maytansine)) to FGly, resulting in the formation of a stable, covalent C—C bond between the cytotoxin payload and the antibody. This C—C bond was expected to be stable to physiologically-relevant conditions encountered by the ADC during circulation and FcRn recycling, e.g., proteases, low pH, and reducing reagents. Antibodies bearing the aldehyde tag may be produced at a variety of locations. Experiments were performed to test the effects of inserting the aldehyde tag at the heavy chain C-terminus (CT). Biophysical and functional characteriziaton was performed on the resulting ADCs made by conjugation to maytansine payloads via a HIPS linker.

### Cloning, Expression, and Purification of Tagged Antibodies

The aldehyde tag sequence was inserted at the heavy chain C-terminus (CT) of the anti-CD25 antibody daclizumab using standard molecular biology techniques. For small-scale production, CHO-S cells were transfected with human FGE expression constructs and pools of FGE-overexpressing cells were used for the transient production of antibodies. For larger-scale production, GPEx technology (Catalent, Inc., Somerset, NJ) was used to generate a clonal cell line overexpressing human FGE (GPEx). Then, the FGE clone was used to generate bulk stable pools of antibody-expressing cells. Antibodies were purified from the conditioned medium using a Protein A chromatography (MabSelect, GE Healthcare Life Sciences, Pittsburgh, PA). Purified antibodies were flash frozen and stored at −80° C. until further use.

### Bioconjugation, Purification, and HPLC Analytics

C-terminally aldehyde-tagged aCD25 antibody (daclizumab, 15 mg/mL) was conjugated to a maytansine payload attached to a HIPS-4AP linker (8 mol. equivalents drug: antibody) for 48-72 h at 37° C. in 20 mM sodium citrate, 50 mM NaCl pH 5.0 containing 0.85% DMA. After conjugation, free drug was removed by tangential flow filtration and the ADC was buffer exchanged into 20 mM sodium citrate, 50 mM NaCl pH 5.5.

To determine the drug-to-antibody ratio (DAR) of the final product, ADCs were examined by analytical HIC (Tosoh #14947) with mobile phase A: 1.5 M ammonium sulfate, 25 mM sodium phosphate pH 7.0, and mobile phase B: 25% isopropanol, 18.75 mM sodium phosphate pH 7.0. To determine aggregation, samples were analyzed using analytical size exclusion chromatography (SEC; Tosoh #08541) with a mobile phase of 300 mM NaCl, 25 mM sodium phosphate pH 6.8.

### Results

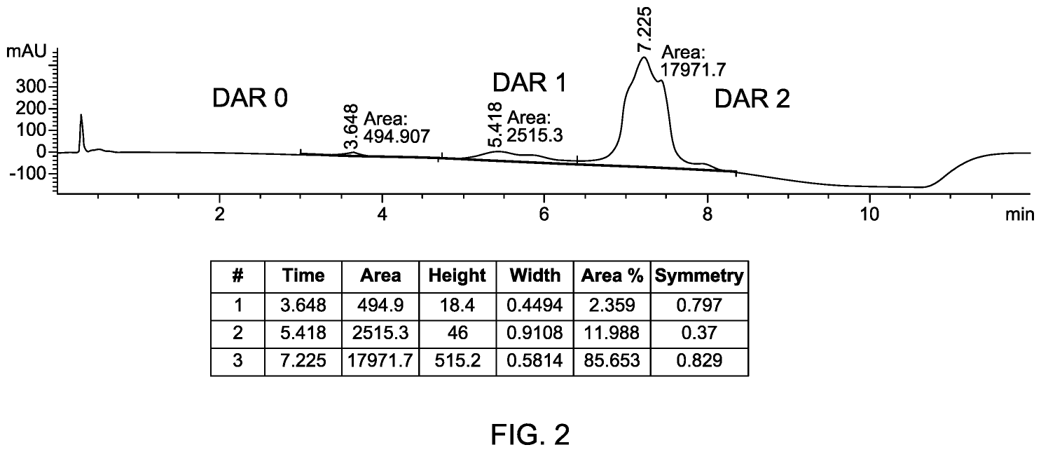

The anti-CD25 antibody modified to contain the aldehyde tag at the heavy chain C-terminus (CT) was conjugated to a maytansine payload attached to a HIPS-4AP linker. Upon completion, remaining free drug was removed during buffer exchange by tangential flow filtration. These reactions were high yielding, with >90% conjugation efficiency and >80% total yield. The resulting ADCs had drug-to-antibody ratios (DARs) of 1.75-1.84 and were predominately monomeric. FIGS. 2-5 document DARs from representative reactions as determined by HIC and reversed phase PLRP chromatography, and show the monomeric integrity as determined by SEC.

FIG. 2 shows a hydrophobic interaction column (HIC) trace of an aldehyde-tagged anti-CD25 antibody conjugated at the heavy chain C-terminus (CT) to a maytansine payload attached to a HIPS-4AP linker. FIG. 2 indicates that the DAR was 1.83 as determined by HIC.

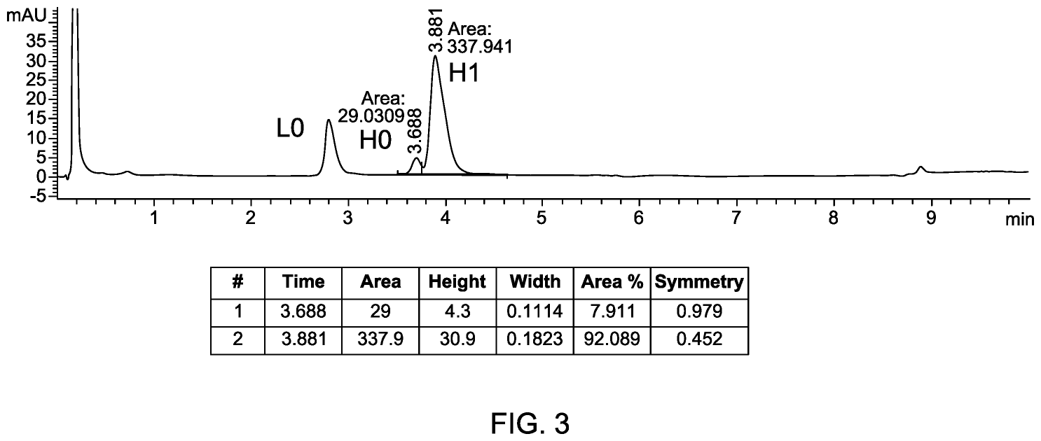

FIG. 3 shows a reversed phase chromatography (PLRP) trace of an aldehyde-tagged anti-CD25 antibody conjugated at the heavy chain C-terminus (CT) to a maytansine payload attached to a HIPS-4AP linker. FIG. 3 indicates that the DAR was 1.81 as determined by PLRP.

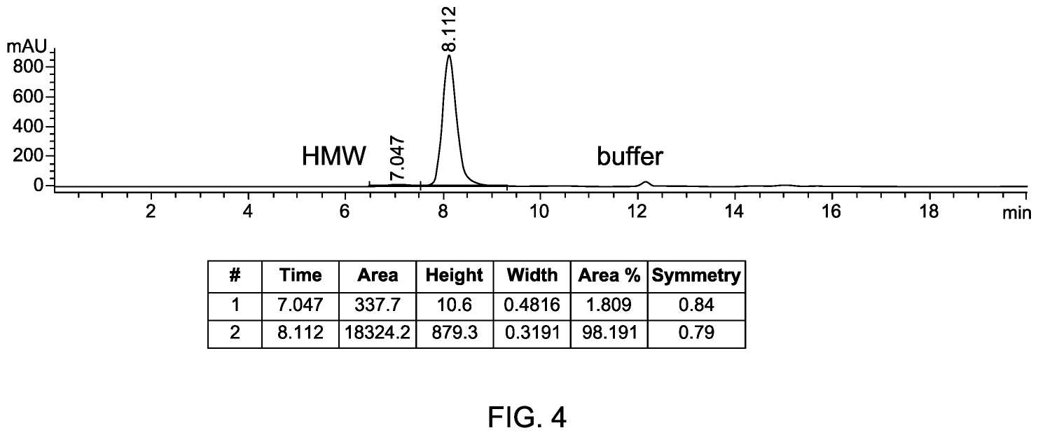

FIG. 4 shows a graph of analytical size exclusion chromatography (SEC) analysis of an aldehyde-tagged anti-CD25 antibody conjugated at the heavy chain C-terminus (CT) to a maytansine payload attached to a HIPS-4AP linker. As shown in FIG. 4, analytical SEC indicated 98.2% monomer for the final product.

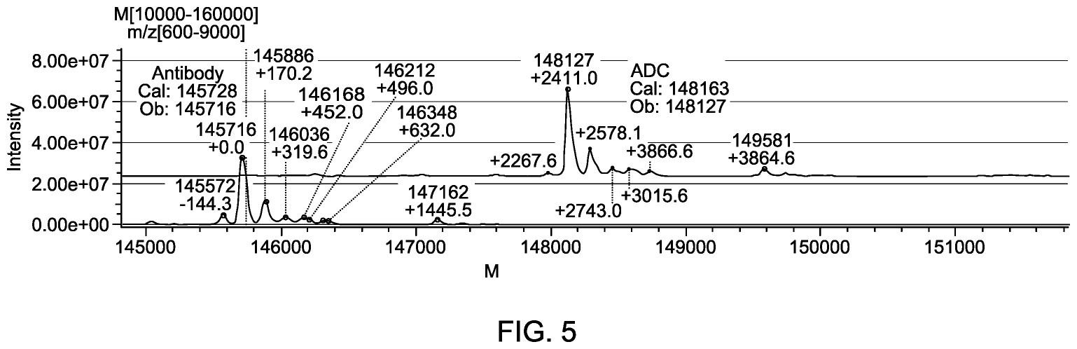

FIG. 5 shows a graph of intact mass analysis of an aldehyde-tagged anti-CD25 antibody conjugated at the heavy chain C-terminus (CT) to a maytansine payload attached to a HIPS-4AP linker. As shown in FIG. 5, a linker/payload-related increase in mass for a DAR2 conjugated species was observed.

### In Vitro Cytotoxicity

The CD25-positive leukemia and lymphoma cell lines, SU-DHL-1, HDLM-2, EoL-1, L-540, and Karpas 299, were obtained from the ATCC and DSMZ cell banks, or were tested at Crown Bioscience, Inc. The cells were maintained in RPMI-1640 medium (Cellgro) supplemented with 10 or 20% fetal bovine serum (Invitrogen) and Glutamax (Invitrogen). 24 h prior to plating, cells were passaged to ensure log-phase growth. On the day of plating, 5000 cells/well were seeded onto 96-well plates in 100 μL normal growth medium supplemented with 10 IU penicillin and 10 μg/mL streptomycin (Cellgro). Cells were treated at various concentrations with 20 μL of diluted analytes, and the plates were incubated at 37° C. in an atmosphere of 5% CO₂. After 5 d, 100 μL/well of Cell Titer-Glo reagent (Promega) was added, and luminescence was measured using a Molecular Devices SpectraMax M5 plate reader. GraphPad Prism software was used for data analysis.

### Results

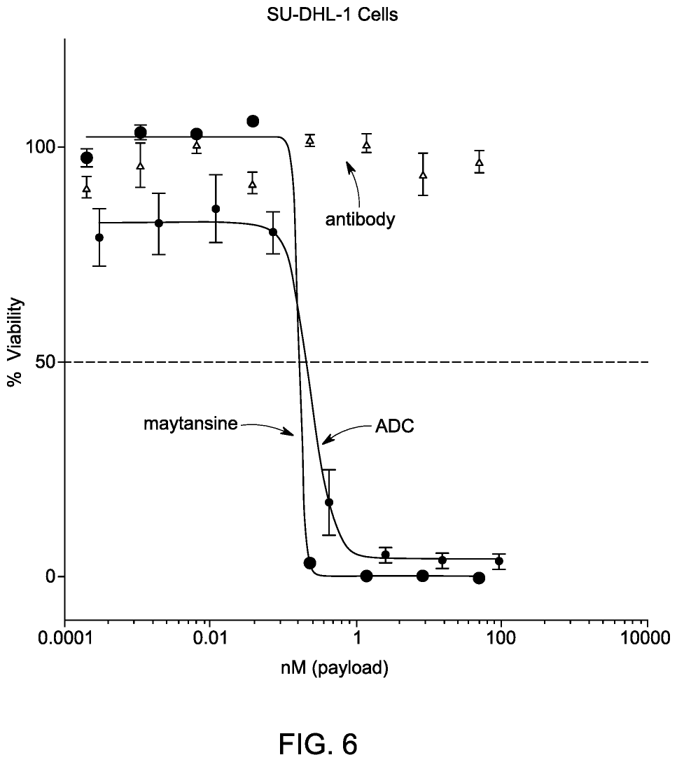

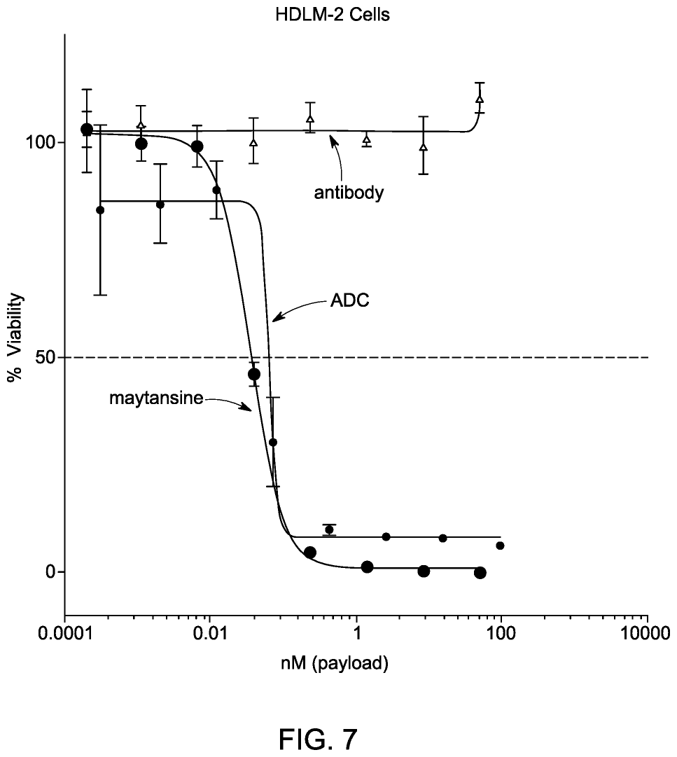

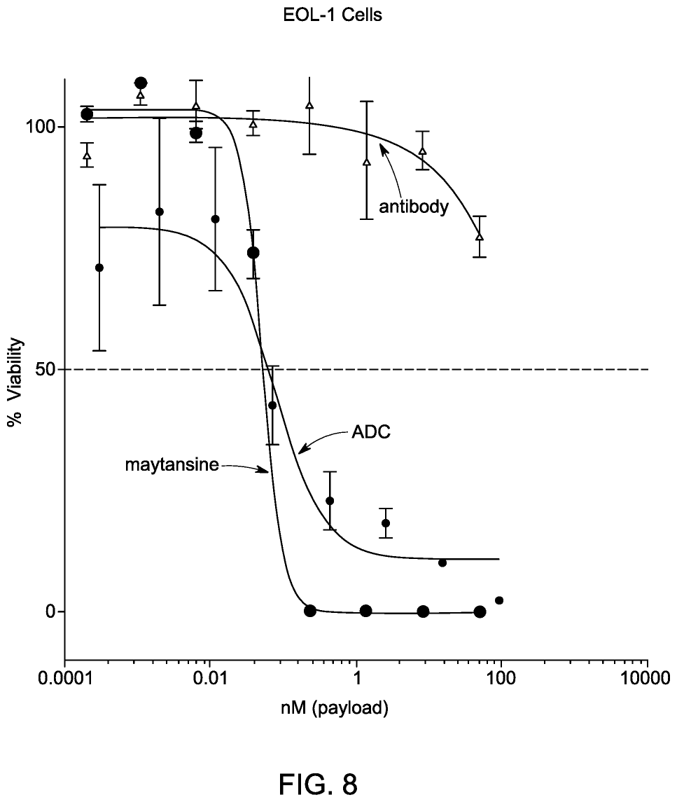

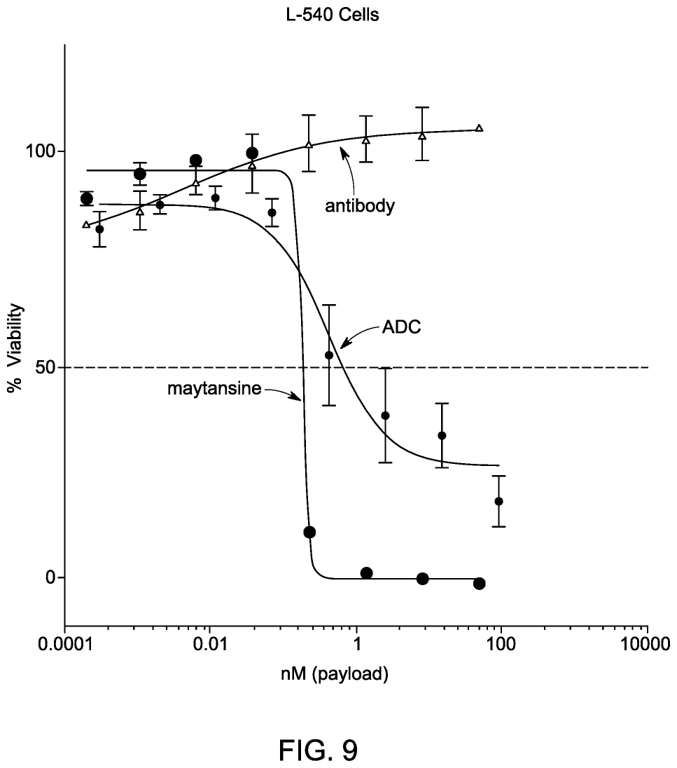

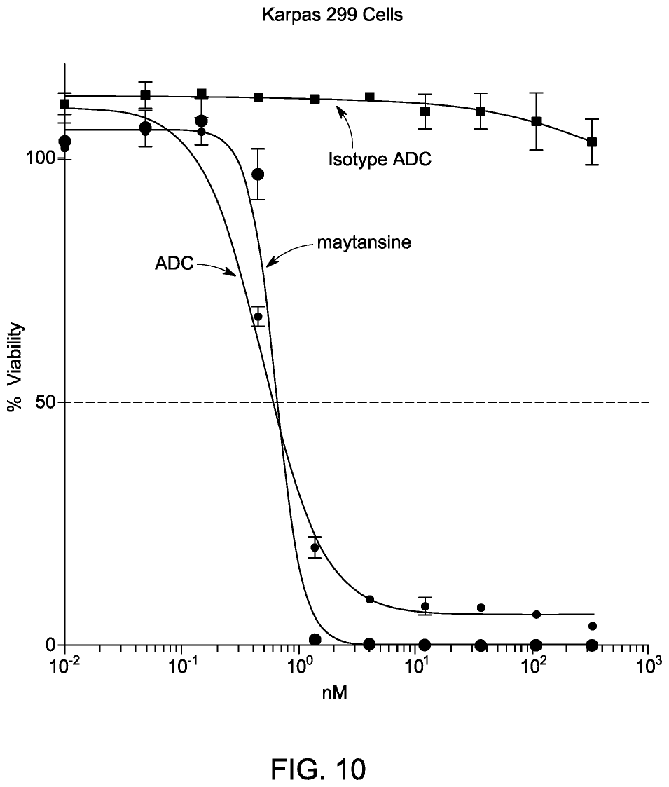

The anti-CD25 HIPS-4AP-maytansine conjugate exhibited very potent activity against all five tested cell lines, comparable to free maytansine. FIGS. 6-10 show the results for the SU-DHL-1 (IC₅₀: 0.24 nM), HDLM-2 (IC₅₀: 0.06 nM), EoL-1 (IC₅₀: 0.07 nM), L-540 (IC₅₀: 0.43 nM), and Karpas 299 (IC₅₀: 0.49 nM) cell lines, respectively. The ADC IC₅₀ concentrations ranged from 0.06 to 0.49 nM in these assays, while the free maytansine IC₅₀ concentrations ranged from 0.03 to 0.19 nM.

### Xenograft Studies

Female CB 17.SCID mice (6/group) were inoculated subcutaneously with 5×10⁶ Karpas 299 cells. Treatment began when the tumors reached an average of 205 mm³, at which time the animals were dosed intravenously with vehicle alone or the anti-CD25 HIPS-4AP-maytansine conjugate (10 mg/kg). One group received a single dose of the anti-CD25 HIPS-4AP-maytansine conjugate, while another group was dosed once per week for a total of four doses (qwk×4). The animals were monitored twice weekly for body weight and tumor size. Animals were euthanized when tumors reached 2000 mm³.

### Results

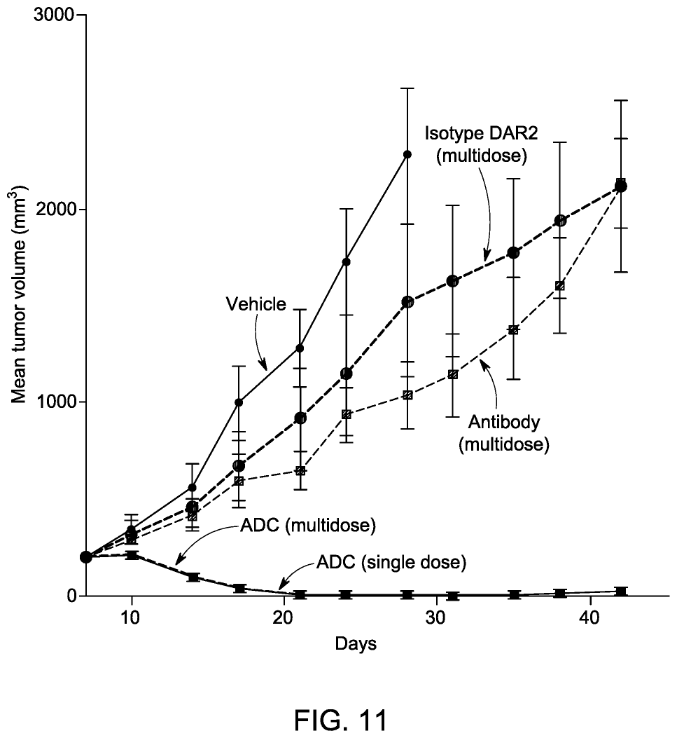

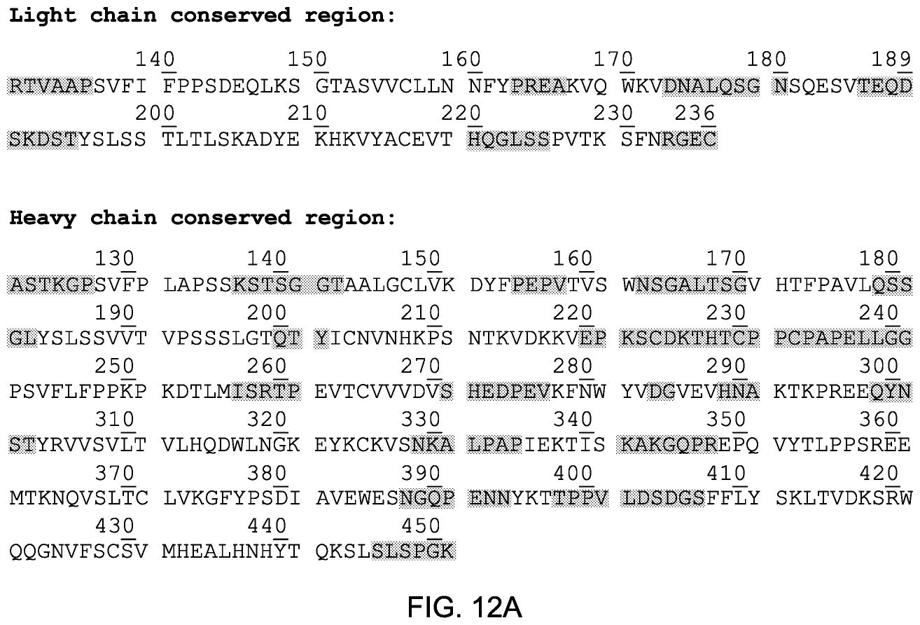

The median time to endpoint for animals in the vehicle control groups was 28 days. At that point, none of the animals that had been dosed with the anti-CD25 HIPS-4AP-maytansine conjugate had detectable tumors. Data is shown in FIG. 11.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-42. (canceled)

43. An anti-CD25 antibody comprising a formylglycine (FGly) residue.

44. The anti-CD25 antibody of claim 43, comprising the sequence:
X¹(FGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the antibody, X¹ is present; and
X² and X³ are each independently any amino acid.

45. The anti-CD25 antibody of claim 44, wherein the sequence is L(FGly)TPSR.

46. The anti-CD25 antibody of claim 44, wherein
Z³⁰ is selected from R, K, H, A, G, L, V, I, and P;
X¹ is selected from L, M, S, and V; and
X² and X³ are each independently selected from S, T, A, V, G, and C.

47. The anti-CD25 antibody of claim 44, wherein the sequence is at a C-terminus of a heavy chain constant region of the anti-CD25 antibody.

48. The anti-CD25 antibody of claim 47, wherein the heavy chain constant region comprises the sequence:
X¹(FGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid,
wherein the sequence is C-terminal to the amino acid sequence SLSLSPG.

49. The anti-CD25 antibody of claim 47, wherein the heavy chain constant region comprises the sequence SPGSL(FGly)TPSRGS.

50. The anti-CD25 antibody of claim 47, wherein
Z³⁰ is selected from R, K, H, A, G, L, V, I, and P;
X¹ is selected from L, M, S, and V; and
X² and X³ are each independently selected from S, T, A, V, G, and C.

51. The anti-CD25 antibody of claim 44, wherein the FGly residue is positioned in a light chain constant region of the anti-CD25 antibody.

52. The anti-CD25 antibody of claim 51, wherein the light chain constant region comprises the sequence:
X¹(FGly)X²Z²⁰X³Z³⁰ (II)
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and
wherein the sequence is C-terminal to the sequence KVDNAL, and/or is N-terminal to the sequence QSGNSQ.

53. The anti-CD25 antibody of claim 52, wherein the light chain constant region comprises the sequence KVDNAL(FGly)TPSRQSGNSQ.

54. The anti-CD25 antibody of claim 52, wherein
Z³⁰ is selected from R, K, H, A, G, L, V, I, and P;
X¹ is selected from L, M, S, and V; and
X² and X³ are each independently selected from S, T, A, V, G, and C.

55. The anti-CD25 antibody of claim 44, wherein the FGly residue is positioned in a heavy chain CH1 region of the anti-CD25 antibody.

56. The anti-CD25 antibody of claim 55, wherein the heavy chain CH1 region comprises the sequence:
X¹(FGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and
wherein the sequence is C-terminal to the amino acid sequence SWNSGA and/or is N-terminal to the amino acid sequence GVHTFP.

57. The anti-CD25 antibody of claim 56, wherein the heavy chain CH1 region comprises the sequence SWNSGAL(FGly)TPSRGVHTFP.

58. The anti-CD25 antibody of claim 56, wherein
Z³⁰ is selected from R, K, H, A, G, L, V, I, and P;
X¹ is selected from L, M, S, and V; and
X² and X³ are each independently selected from S, T, A, V, G, and C.

59. The anti-CD25 antibody of claim 44, wherein the FGly residue is positioned in a heavy chain CH2 region of the anti-CD25 antibody.

60. The anti-CD25 antibody of claim 44, wherein the FGly residue is positioned in a heavy chain CH3 region of the anti-CD25 antibody.

61.-64. (canceled)

65. The anti-CD25 antibody of claim 44, wherein the anti-CD25 antibody comprises:
a heavy chain comprising complementarity determining regions (CDRs) of SYRMH (SEQ ID NO: 6), YINPSTGYTEYNQKFKD (SEQ ID NO: 7), and GGGVFDY (SEQ ID NO: 8); and
a light-chain comprising CDRs of SASSSISYMH (SEQ ID NO: 9), YTSILHS (SEQ ID NO: 10), and QQGNTLPWT (SEQ ID NO: 11).

66. The anti-CD25 antibody of claim 44, wherein the anti-CD25 antibody is daclizumab.
